(19) United States
(12) Patent Application Publication
(10) Pub. No.: US 2009/0012069 A1
(43) Pub. Date: Jan. 8, 2009

(54) Novel Antidiabetic Compounds

(76) Inventors: Braj Bhushan Lohray, Gujarat (IN); Vidya Bhushan Lohray, Gujarat (IN); Harikishore Pingali, Gujarat (IN)

Correspondence Address: NIXON & VANDERHYE, PC, 901 NORTH GLEBE ROAD, 11TH FLOOR, ARLINGTON, VA, 22203, US

(21) Appl. No.: 11/658,990

(22) PCT Filed: Jun. 24, 2005
(86) PCT No.: PCT/IN05/00215

§ 371 Date: Aug. 17, 2007

(30) Foreign Application Priority Data
Aug. 20, 2004 (IN) 901/MUM/2004

(51) Int. Cl. C07D 319/06; C07D 405/12; C07D 405/14; C07D 409/12; C07D 413/12; C07D 413/14; C07D 417/12; A61K 31/422; A61K 31/4025; A61K 31/404; A61K 31/517; A61K 31/536; A61K 31/5415; A61K 31/366; A61P 3/04; A61P 3/06; A61P 3/10

(52) U.S. Cl. 514/225.2; 548/235; 514/374; 514/343; 546/276.4; 546/282.4; 514/336; 514/266.24; 544/283; 544/105; 514/230.5; 514/365; 548/202; 544/35; 544/51; 514/224.2; 514/229.8; 544/102; 549/369; 514/452

(57) ABSTRACT

Disclosed are novel compounds of general formula (I) where the symbols are as defined in the specification, their tautomeric forms, their stereoisomers, their pharmaceutically acceptable salts, pharmaceutical compositions containing them, methods for their preparation, use of these compounds in medicine and the intermediates involved in their preparation. The compounds are useful in the treatment of diabetes and related diseases.

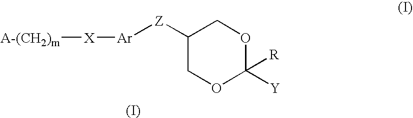

The present invention relates to novel heterocyclic compounds of the general formula (I), their tautomeric forms, their stereoisomers, their pharmaceutically acceptable salts, pharmaceutical compositions containing them, methods for their preparation, use of these compounds in medicine and the intermediates involved in their preparation.

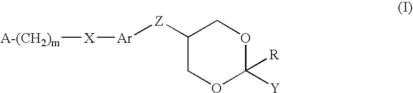

The compounds of the general formula (I) lower blood glucose, lower or modulate triglyceride levels and/or cholesterol levels and/or low-density lipoproteins (LDL) and raises the high-density lipoproteins (HDL) plasma levels and hence are useful in combating different medical conditions, where such lowering (and raising) is beneficial. Thus, it could be used in the treatment and/or prophylaxis of obesity, hyperlipidaemia, hypercholesteremia, hypertension, atherosclerotic disease events, vascular restenosis, diabetes and many other related conditions.

The compounds of general formula (I) are useful to prevent or reduce the risk of developing atherosclerosis, which leads to diseases and conditions such as arteriosclerotic cardiovascular diseases, stroke, coronary heart diseases, cerebrovascular diseases, peripheral vessel diseases and related disorders. These compounds of general formula (I) are useful for the treatment and/or prophylaxis of metabolic disorders loosely defined as Syndrome X. The characteristic features of Syndrome X include initial insulin resistance followed by hyperinsulinemia, dyslipidemia and impaired glucose tolerance. The glucose intolerance can lead to non-insulin dependent diabetes mellitus (NIDDM, Type 2 diabetes), which is characterized by hyperglycemia, which if not controlled may lead to diabetic complications or metabolic disorders caused by insulin resistance. Diabetes is no longer considered to be associated only with glucose metabolism, but it affects anatomical and physiological parameters, the intensity of which vary depending upon stages/duration and severity of the diabetic state. The compounds of this invention are also useful in prevention, halting or slowing progression or reducing the risk of the above mentioned disorders along with the resulting secondary diseases such as cardiovascular diseases, like arteriosclerosis, atherosclerosis; diabetic retinopathy, diabetic neuropathy and renal disease including diabetic nephropathy, glomerulonephritis, glomerular sclerosis, nephrotic syndrome, hypertensive nephrosclerosis and end stage renal diseases, like microalbuminuria and albuminuria, which may be result of hyperglycemia or hyperinsulinemia.

### BACKGROUND OF THE INVENTION

Hyperlipidaemia has been recognized as the major risk factor in causing cardiovascular diseases due to atherosclerosis. Atherosclerosis and other such peripheral vascular diseases affect the quality of life of a large population in the world. The therapy aims to lower the elevated plasma LDL cholesterol, low-density lipoprotein and plasma triglycerides in order to prevent or reduce the risk of occurrence of cardiovascular diseases. The detailed etiology of atherosclerosis and coronary artery diseases is discussed by Ross and Glomset [New Engl. J. Med., 295, 369-377 (1976)]. Plasma cholesterol is generally found esterified with various serum lipoproteins and numerous studies have suggested an inverse relationship between serum HDL-cholesterol level and risk for occurrence of cardiovascular disease. Many studies have suggested an increased risk of coronary artery diseases (CAD) due to elevated LDL and VLDL-cholesterol levels [Stampfer et al., N. Engl. J. Med., 325, 373-381 (1991)]. The other studies illustrate protective effects of HDL against progression of atherosclerosis. Thus, HDL has become a crucial factor in treating diseases with increased levels of cholesterol [Miller el. al., Br. Med. J. 282, 1741-1744 (1981); Picardo et al., Arteriosclerosis, 6, 434-441 (1986); Macikinnon et al., J. Biol. Chem. 261, 2548-2552 (1986)].

Diabetes is associated with a number of complications and also affect a large population. This disease is usually associated with other diseases such as obesity, hyperlipidemia, hypertension and angina. It is well established that improper treatment can aggravate impaired glucose tolerance and insulin resistance, thereby leading to frank diabetes. Further, patients with insulin resistance and type 2 diabetes often have raised triglycerides and low HDL-cholesterol concentrations and therefore, have greater risk of cardiovascular diseases. The present therapy for these diseases includes sulfonylureas and biguanides along with insulin. This type of drug therapy may lead to mild to severe hypoglycemia, which may lead to coma or in some cases may lead to death, as a result of unsatisfactory glycaemic control by these drugs. Recent addition of drugs in the treatment of diabetes are the thiazolidinediones, drugs having insulin-sensitizing action Thiazolidinediones like troglitazone, rosiglitazone and pioglitazone are prescribed alone or in combination with other anti-diabetic agents.

These are useful in treating diabetes, lipid metabolism but are suspected to have tumor-inducing potential and cause hepatic dysfunction, which may lead to liver failure. Further, serious undesirable side-effects have occurred in animal and/or human studies which include cardiac hypertrophy, hema dilution and liver toxicity in a few glitazones progressing to advanced human trials. The drawback is considered to be idiosyncratic. Presently, there is a need for a safe and an effective drug, to treat insulin resistance, diabetes and hyperlipidemia [Exp. Clin. Endocrinol. Diabetes: 109(4), S548-9 (2001)]

Obesity is another major health problem being associated with increased morbidity and mortality. It is a metabolic disorder, in which excess of fat is accumulated in the body. Although, its etiology is unclear, the general feature includes excess of calorie intake than it is consumed. Various therapies such as dieting, exercise, appetite suppression, inhibition of fat absorption etc. have been used to combat obesity. However, more efficient therapies to treat this abnormality is essential as obesity is closely related to several diseases such as coronary heart disease, stroke, diabetes, gout, osteoarthritis, hyperlipidaemia and reduced fertility. It also leads to social and psychological problems [Nature Reviews: Drug Discovery: 1(4), 276-86 (2002)].

Peroxisome Proliferator Activated Receptor (PPAR) is a member of the steroid/retinoid/thyroid hormone receptor family. PPAR∝, PPARγ and PPARδ have been identified as subtypes of PPARs. Extensive reviews regarding PPAR, their role in different diseased conditions are widely published [Endocrine Reviews, 20(5), 649-699 (1999); J. Medicinal Chemistry, 43(4), 58-550 (2000); Cell, 55, 932-943 (1999); Nature, 405, 421-424 (2000); Trends in Pharmacological Sci., 469-473 (2000)]. PPARγ activation has been found to play a central role in initiating and regulating adipocyte differentiation [Endocrinology 135, 798-800, (1994)] and energy homeostasis, [Cell, 83, 803-812 (1995); Cell, 99, 239-242 (1999)]. PPARγ agonists would stimulate the terminal differentiation of adipocyte precursors and cause morphological and molecular changes characteristic of a more differentiated, less malignant state. During adipocyte differentiation, several highly specialized proteins are induced, which are being involved in lipid storage and metabolism. It is accepted that PPARPγ activation leads to expression of CAP gene [Cell Biology, 95, 14751-14756, (1993)], however, the exact link from PPARγ activation to changes in glucose metabolism and decrease in insulin resistance in muscle has not been clear. PPARα is involved in stimulating β-oxidation of fatty acids [Trends Endocrine. Metabolism, 4, 291-296 (1993)] resulting in plasma circulating free fatty acid reduction [Current Biol., 5, 618-621 (1995)]. Recently, role of PPAR-Y activation in the terminal differentiation of adipocyte precursors has been implicated in the treatment of cancer. [Cell, 79, 1147-1156 (1994); Cell, 377-389 (1996); Molecular Cell, 465-470 (1998); Carcinogenesis, 1949-1953 (1998); Proc. Natl. Acad. Sci., 94, 237-241 (1997); Cancer Research, 58, 3344-3352 (1998)]. Since PPARγ is expressed in certain cells consistently, PPARγ agonists would lead to nontoxic chemotherapy. There is growing evidence that PPAR agonists may also influence the cardiovascular system through PPAR receptors as well as directly by modulating vessel wall function [Med. Res. Rev., 20 (5), 350-366 (2000)].

PPAR α agonists have been found useful in the treatment of obesity (WO 97/36579). Dual PPAR α and γ agonists have been suggested to be useful for Syndrome X (WO 97/25042). PPAR γ agonists and HAG-CoA reductase inhibitors have exhibited synergism and indicated the usefulness of the combination in the treatment of atherosclerosis and xanthoma (EP 0753 298).

Most recently PPAR delta was reported to modulate lipid metabolism in which PPAR delta serves as a widespread regulator of fat burning. In vitro activation of PPAR delta in adipocytes and skeletal muscle cells promotes fatty acid oxidation and utilization. It has also been reported that PPAR delta deficient mice challenged with high-fat diet show reduced energy uncoupling and are prone to obesity (Wang Y X et. al., Cell (2003), 113(2), 159-170). The transcriptional repression of atherogenic inflammation by ligand-activated PPAR delta was also reported, which further indicates the importance of PPAR delta in combating cardiovascular diseases (Lee, C H et al., Science 302, 453-457, 2003).

Leptin is a protein when bound to leptin receptors is involved in sending satiety signal to the hypothalamus. Leptin resistance would therefore lead to excess food in-take, reduced energy expenditure, obesity, impaired glucose tolerance and diabetes [Science, 269, 543-46 (1995)]. It has been reported that insulin sensitizers lower plasma leptin concentration [Proc. Natl. Acad. Sci. 93, 5793-5796 (1996): WO 98/02159)].

Several compounds have been reported which are dual agonists of PPAR α and γ like alkoxy phenyl propanoic acid derivatives, aryloxy propanoic acid derivatives, benzyl glycine derivatives etc have been reported and are in various developmental stages.

US 20030166697 (Nippon Shinayaku) discloses compounds of the following general formula:

R₁-Het-D-E

wherein

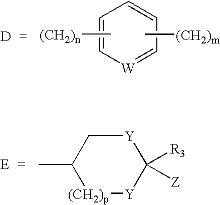

R₁ represents (un)substituted aryl, aromatic heterocyclic or cycloalkyl groups; ‘Het’ is an optionally substituted divalent aromatic heterocyclic group; W is —CH— or N; m=1-10; n=0-9; p=0-2; Y=O or S; R₃ is H or alkyl; Z=carboxy, alkoxy carbonyl etc.

WO 2000004011 discloses compounds having the following general formula for the treatment of dyslipidemia, atherosclerosis and diabetes;

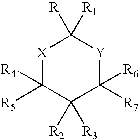

where X, Y=CH₂, O, S, NRa (Ra=H, alkyl aryl, etc.); R=H, alkyl, cycloalkyl, etc.; R¹=H, alkyl, hydroxyalkyl, —(CH₂)ₜ—COORc where t=0-6 & Rc represents H or alkyl group, etc.; R₂ & R³=H, alkyl cycloalkyl, (C₆-C₁₀)aryl, (C₆-C₁₀)aryl(C₁-C₇)alkyl, 3-10 membered optionally substituted heterocyclic group etc.; or R₂ & R₃ optionally form a chain —(CH2)ᵣ₁ (r=2-5), etc.; R₄-R₇=H, alkyl, (un)substituted aryl, etc.

However, the therapeutic potential of these compounds to treat diseases has not yet been proved and so there remains the need to develop newer medicines which are better or of comparable efficacy with the present treatment regimes, have lesser side effects and require a lower dosage regime

Surprisingly, we have found that the novel compounds of formula (I) are useful as hypocholesterolemic, hypolipidaemic, hypolipoproteinemic, anti-obesity and antihyperglycemic agents which may have additional body weight lowering effect and beneficial effect in the treatment and/or prophylaxis of diseases caused by hyperlipidaemia, diseases classified under Syndrome X and atherosclerosis, and methods for their preparation. Also surprisingly, the compounds of formula (I) have been found to be useful as hypocholesterolemic, hypolipidaemic, hypolipoproteinemic, anti-obesity and antihyperglycemic agents with reduced side effects. Also, the compounds showed preferable affinity towards PPAR subtypes.

### PREFERRED EMBODIMENTS OF THE INVENTION

In an embodiment of the present invention is provided novel substituted heterocyclic compounds represented by the general formula (I), their tautomeric forms, their stereoisomers, their pharmaceutically acceptable salts, and pharmaceutical compositions containing them or their mixtures thereof.

In another embodiment of the present invention is provided a process for the preparation of novel substituted heterocyclic compounds represented by the general formula (I), their tautomeric forms, their stereoisomers, their pharmaceutically acceptable salts.

In a further embodiment of the present invention is provided pharmaceutical compositions containing compounds of the general formula (I), their tautomeric forms, their stereoisomers, their pharmaceutically acceptable salts, or their mixtures in combination with suitable carriers, solvents, diluents and other media normally employed in preparing such compositions.

### DESCRIPTION OF THE INVENTION

Accordingly, the present invention provides novel compounds of the general formula (I),

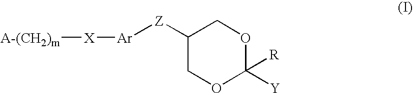

their tautomeric forms, their stereoisomers, their pharmaceutically acceptable salts, wherein
‘A’ represents an optionally substituted single or fused group selected from aryl, heteroaryl, heterocyclyl groups; or

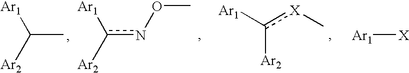

wherein ‘Ar’, ‘Ar₁’ and Ar₂′ may be the same or different and independently represents an optionally substituted single or fused aryl, heteroaryl or a heterocyclic group;
‘X’ represents oxygen, sulfur or nitrogen;
‘Y’ represents COOR¹, CONR¹R²;
Z represents a bond or —CH₂—;
‘m’ is an integer from 1-3;
R, R¹ & R² may be same or different and independently represents hydrogen, optionally substituted groups selected from linear or branched alkyl or aryl groups.

When any one of ‘A’, ‘Ar₁’ or ‘Ar₂’ is substituted, the substituents may be selected from hydroxy, oxo, halo, thio, nitro, amino, cyano, formyl, or optionally substituted groups selected from amidino, hydrazino, alkyl, haloalkyl, perhaloalkyl, alkoxy, haloalkoxy, perhaloalkoxy, alkenyl, alkynyl, cycloalkyl, cycloalkenyl, bicycloalkyl, bicycloalkenyl, alkoxy, alkenoxy, cycloalkoxy, aryl, aryloxy, aralkyl, aralkoxy, heterocyclyl, heteroaryl, heterocyclylalkyl, heteroaralkyl, heteroaryloxy, heteroaralkoxy, heterocyclyloxy, heterocyclylalkoxy, heterocyclylalkoxyacyl, acyl, acyloxy, acylamino, monosubstituted or disubstituted amino, arylamino, aralkylamino, carboxylic acid and its derivatives such as esters and amides, carbonylamino, hydroxyalkyl, aminoalkyl, alkoxyalkyl, aryloxyalkyl, aralkoxyalkyl, alkylthio, thioalkyl, arylthio, alkylsulfonylamino, alkylsulfonyloxy, alkoxycarbonylamino, aryloxycarbonylamino, aralkyloxycarbonylamino, aminocarbonylamino, alkylaminocarbonylamino, alkoxyamino, hydroxylamino, sulfenyl derivatives, sulfonyl derivatives, sulfonic acid and its derivatives; preferably the substituents may be selected from hydroxy, halo, oxo, or optionally substituted groups selected from alkyl, monosubstituted or disubstituted amino, alkoxy, acyl, aryl, aryloxy, aralkyl, aralkoxy, heterocyclyl, heteroaryl, heterocyclylalkyl, heteroaralkoxy, heterocyclyloxy, alkylthio, arylthio, alkylsulfonylamino, alkylsulfonyloxy, carboxylic acid and its derivatives such as esters and amides,

The substituents on ‘A’, ‘Ar₁’ or ‘Ar₂’ may further be optionally substituted by any of the groups as mentioned above;

When the groups representing ‘Ar’ are substituted, the substituents may be selected from halogen, optionally substituted groups selected from linear or branched alkyl, alkoxy, thioalkyl, haloalkyl, haloalkoxy, acyl, arylaminoalkyl, aminoalkyl groups.

In a preferred embodiment the groups, radicals described above may be selected from:
the “alkyl” group used either alone or in combination with other radicals, denotes a linear or branched radical containing one to eight carbons, selected from methyl, ethyl, n-propyl, iso-propyl, n-butyl, sec-butyl, tert-butyl, amyl, t-amyl, n-pentyl, 17-hexyl, iso-hexyl, heptyl, octyl and the like;
the “alkenyl” group used either alone or in combination with other radicals, is selected from a radical containing from two to twelve carbons, more preferably groups selected from vinyl, allyl, 2-butenyl, 3-butenyl, 2-pentenyl; 3-pentenyl, 4-pentenyl, 2-hexenyl, 3-hexenyl, 4-hexenyl, 5-hexenyl, 2-heptenyl, 3-heptenyl, 4-heptenyl, 5-heptenyl, 6-heptenyl and the like; the “alkenyl” group includes dienes and trienes of straight and branched chains;
the “alkynyl” group used either alone or in combination with other radicals, is selected from a linear or branched radical containing two to twelve carbon atoms, more preferably thynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 3-hexynyl, 4-hexynyl, 5-hexynyl, and the like. The term “alkynyl” includes di- and tri-ynes;
the “cycloalkyl” group used either alone or in combination with other radicals, is selected from a radical containing three to seven carbons, more preferably cyclopropyl, cyclobutyl, cyclopentyl, cyclohexyl, cycloheptyl and the like;
the “cycloalkenyl” group used either alone or in combination with other radicals, are preferably selected from cyclopropenyl, 1-cyclobutenyl, 2-cyclobutenyl, 1-cyclopentenyl, 2-cyclopentenyl, 3-cyclopentenyl, 1-cyclohexenyl, 2-cyclohexenyl, 3-cyclohexenyl, 1-cycloheptenyl, cycloheptadienyl, cycloheptatrienyl, and the like;
the “alkoxy” group used either alone or in combination with other radicals, is selected from groups containing an alkyl radical, as defined above, attached directly to an oxygen atom, more preferably groups selected from methoxy, ethoxy, n-propoxy, iso-propoxy, n-butoxy, t-butoxy, iso-butoxy, pentyloxy, hexyloxy, and the like;
the “alkenoxy” group used either alone or in combination with other radicals, is selected from groups containing an alkenyl radical, as defined above, attached to an oxygen atom, more preferably selected from vinyloxy, allyloxy, butenoxy, pentenoxy, hexenoxy, and the like;
the “cycloalkoxy” group used either alone or in combination with other radicals, is selected from groups containing a cycloalkyl radical as defined above, attached directly to an oxygen atom, more preferably selected from cyclopropyloxy, cyclobutyloxy, cyclopentyloxy, cyclohexyloxy, cycloheptyloxy and the like.
the “halo” or “halogen” group used either alone or in combination with other radicals, such as “haloalkyl”, “perhaloalkyl” etc. is selected from fluoro, chloro, bromo or iodo group;
the “haloalkyl” group is selected from an alkyl radical, as defined above, suitably substituted with one or more halogens; such as perhaloalkyl, more preferably, perfluoro(C₁-C₆)alkyl such as fluoromethyl, difluoromethyl, trifluoromethyl, fluoroethyl, difluoroethyl, trifluoroethyl, mono or polyhalo substituted methyl, ethyl, propyl, butyl, pentyl or hexyl groups;
the “haloalkoxy” group is selected from suitable haloalkyl, as defined above, directly attached to an oxygen atom, more preferably groups selected from fluoromethoxy, chloromethoxy, fluoroethoxy chloroethoxy and the like;
the “perhaloalkoxy” group is selected from a suitable perhaloalkyl radical, as defined above, directly attached to an oxygen atom, more preferably groups selected from trifluoromethoxy, trifluoroethoxy, and the like;
the “aryl” or “aromatic” group used either alone or in combination with other radicals, is selected from a suitable aromatic system containing one, two or three rings wherein such rings may be attached together in a pendant manner or may be fused, more preferably the groups are selected from phenyl, naphthyl, tetrahydronaphthyl, indane, biphenyl, and the like;
the ‘aralkyl” group is selected from suitable aryl group as defined above attached to an alkyl group as defined above, more preferably selected from benzyl, phenethyl, naphthylmethyl, and the like;
the “aryloxy” group is selected from a suitable aryl radical, as defined above, attached to a suitable alkoxy group, as defined above, more preferably the groups are selected from phenoxy, naphthyloxy and the like, which may be substituted;
the “aralkoxy” group is selected from a suitable arylalkyl group, as defined above, attached to an oxygen atom, more preferably the groups are selected from benzyloxy, phenethyloxy, naphthylmethyloxy, phenylpropyloxy, and the like, which may be substituted;
the “heterocyclyl” or “heterocyclic” group used either alone or in combination with other radicals, is selected from suitable saturated, partially saturated or unsaturated aromatic or non aromatic mono, bi or tricyclic radicals, containing one or more heteroatoms selected from nitrogen, sulfur and oxygen, more preferably selected from aziridinyl, azetidinyl, pyrrolidinyl, imidazolidinyl, piperidinyl, piperazinyl, 2-oxopiperidinyl, 4-oxopiperidinyl, 2-oxopiperazinyl, 3-oxopiperazinyl, morpholinyl, thiomorpholinyl, 2-oxomorpholinyl, azepinyl, diazepinyl, oxapinyl, thiazepinyl, oxazolidinyl, thiazolidinyl, dihydrothiophene, dihydropyran, dihydrofuran, dihydrothiazole, benzopyranyl, benzopyranonyl, benzodihydrofuranyl, benzodihydrothienyl, pyrazolopyrimidonyl, azaquinazolinoyl, thienopyrimidonyl, quinazolonyl, pyrimidonyl, benzoxazinyl, benzoxazinonyl, benzothiazinyl, benzothiazinonyl, thieno piperidinyl, and the like;
the “heteroaryl” or “heteroaromatic” group used either alone or in combination with other radicals, is selected from suitable single or fused mono, bi or tricyclic aromatic heterocyclic radicals containing one or more hetero atoms selected from O, N or S, more preferably the groups are selected from pyridyl, thienyl, furyl, pyrrolyl, oxazolyl, thiazolyl, isothiazolyl, imidazolyl, isoxazolyl, oxadiazolyl, thiadiazolyl, triazolyl, tetrazolyl, benzofuranyl, benzothienyl, indolinyl, indolyl, azaindolyl, azaindolinyl, pyrazolopyrimidinyl, azaquinazolinyl, pyridofuranyl, pyridothienyl, thienopyrimidyl, quinolinyl, pyrimidinyl, pyrazolyl, quinazolinyl, pyridazinyl, triazinyl, benzimidazolyl, benzotriazolyl, phthalazynil, naphthylidinyl, purinyl, carbazolyl, phenothiazinyl, phenoxazinyl, benzoxazolyl, benzothiazolyl and the like;
the “heterocyclylalkyl” group used either alone or in combination with other radicals, is selected from a suitable heterocyclyl group, as defined above, substituted with a suitable alkyl group as defined above, more preferably the groups are selected from pyrrolidinealkyl, piperidinealkyl, morpholinealkyl, thiomorpholinealkyl, oxazolinealkyl, and the like, which may be substituted;
the “heteroaralkyl” group used either alone or in combination with other radicals, is selected from a suitable heteroaryl group, as defined above, attached to a straight or branched saturated carbon chain containing 1 to 6 carbons, more preferably the groups are selected from (2-furyl)methyl, (3-furyl)methyl, (2-thienyl)methyl, (3-thienyl)methyl, (2-pyridyl)methyl, 1-methyl-1-(2-pyrimidyl)ethyl and the like;
the groups “heteroaryloxy”, “heteroaralkoxy”, “heterocycloxy”, “heterocylylalkoxy” are selected from suitable heteroaryl, heteroarylalkyl, heterocyclyl, heterocyclylalkyl groups respectively, as defined above, attached to an oxygen atom;
the “acyl” group used either alone or in combination with other radicals, is selected from a radical containing one to eight carbons, more preferably selected from formyl, acetyl, propanoyl, butanoyl, iso-butanoyl, pentanoyl, hexanoyl, heptanoyl, benzoyl and the like, which may be substituted;
the “acyloxy” group used either alone or in combination with other radicals, is selected from a suitable acyl group, as defined above, directly attached to an oxygen atom, more preferably such groups are selected from acetyloxy, propionyloxy, butanoyloxy, iso-butanoyloxy, benzoyloxy and the like;
the “acylamino” group used either alone or in combination with other radicals, is selected from a suitable acyl group as defined earlier, attached to an amino radical, more preferably such groups are selected from CH₃CONH, C₂H₅CONH, C₃H₇CONH, C₄H₉CONH, C₆H₅CONH and the like, which may be substituted;
the “mono-substituted amino” group used either alone or in combination with other radicals, represents an amino group substituted with one group selected from (C₁-C₆)alkyl, substituted alkyl, aryl, substituted aryl or arylalkyl groups as defined earlier, more preferably such groups are selected from methylamine, ethylamine, n-propylamine, n-butylamine, n-pentylamine and the like;
the “disubstituted amino” group used either alone or in combination with other radicals, represents an amino group, substituted with two radicals that may be same or different selected from (C₁-C₆)alkyl, substituted alkyl aryl, substituted aryl, or arylalkyl groups, as defined above, more preferably the groups are selected from dimethylamino, methylethylamino, diethylamino, phenylmethyl amino and the like;
the “arylamino” used either alone or in combination with other radicals, represents an aryl group, as defined above, linked through amino having a free valence bond from the nitrogen atom, more preferably the groups are selected from phenylamino, naphthylamino, N-methyl anilino and the like;
the “aralkylamino” used either alone or in combination with other radicals, represents an arylalkyl group as defined above linked through an amino group having a free valence bond from the nitrogen atom, more preferably selected from benzylamino, phenethylamino, 3-phenylpropylamino, 1-napthylmethylamino, 2-(1-napthyl)ethylamino and the like;
the “oxo” or “carbonyl” group used either alone (—C═O—) or in combination with other radicals such as alkyl described above, for e.g. “alkylcarbonyl”, denotes a carbonyl radical (—C—O—) substituted with an alkyl radical described above such as acyl or alkanoyl;
the “carboxylic acid” group, used alone or in combination with other radicals, denotes a —COOH group, and includes derivatives of carboxylic acid such as esters and amides;
the “ester” group used alone or in combination with other radicals, denotes —COO— group, and includes carboxylic acid derivatives, more preferably the ester moieties are selected from alkoxycarbonyl, such as methoxycarbonyl, ethoxycarbonyl, and the like, which may optionally be substituted; aryloxycarbonyl group such as phenoxycarbonyl, naphthyloxycarbonyl, and the like, which may optionally be substituted; aralkoxycarbonyl group such as benzyloxycarbonyl, phenethyloxycarbonyl, napthylmethoxycarbonyl, and the like, which may optionally be substituted; heteroaryloxycarbonyl, heteroaralkoxycarbonyl, wherein the heteroaryl group, is as defined above, which may optionally be substituted; heterocyclyloxycarbonyl, where the heterocyclic group, as defined earlier, which may optionally be substituted;
the “amide” group used alone or in combination with other radicals, represents an aminocarbonyl radical (H₂N—C═O—), wherein the amino group is mono- or di-substituted or unsubstituted, more preferably the groups are selected from methylamide, dimethylamide, ethylamide, diethylamide, and the like;
the “aminocarbonyl” group used either alone or in combination with other radicals, may be selected from ‘aminocarbonyl’, “aminocarbonylalkyl”, “n-alkylaminocarbonyl”, “N-arylaminocarbonyl”, “N,N-dialkylaminocarbonyl”, “N-alkyl-N-arylaminocarbonyl”, “N-alkyl-N-hydroxyaminocarbonyl”, and “N-alkyl-N-hydroxyaminocarbonylalkyl”, each of them being optionally substituted. The terms “N-alkylaminocabonyl” and “N,N-dialkylaminocarbonyl” denotes aminocarbonyl radicals, as defined above, which have been substituted with one alkyl radical and with two alkyl radicals, respectively. Preferred are “lower alkylaminocarbonyl” having lower alkyl radicals as described above attached to aminocarbonyl radical. The terms “N-arylaminocarbonyl” and “N-alkyl-N-arylaminocarbonyl” denote aminocarbonyl radicals substituted, respectively, with one aryl radical, or one alkyl, and one aryl radical. The term “aminocarbonylalkyl” includes alkyl radicals substituted with aminocarbonyl radicals;
the “hydroxyalkyl” group used either alone or in combination with other radicals, is selected from an alkyl group, as defined above, substituted with one or more hydroxy radicals, more preferably the groups are selected from hydroxymethyl, hydroxyethyl, hydroxypropyl, hydroxybutyl, hydroxypentyl, hydroxyhexyl and the like;
the “aminoalkyl” group used alone or in combination with other radicals, denotes an amino (—NH₂) moiety attached to an alkyl radical, as defined above, which may be substituted, such as mono- and di-substituted aminoalkyl. The term “alkylamino” used herein, alone or in combination with other radicals, denotes an alkyl radical, as defined above, attached to an amino group, which may be substituted, such as mono- and di-substituted alkylamino;
the “alkoxyalkyl” group used alone or in combination with other radicals, denotes an alkoxy group, as defined above, attached to an alkyl group as defined above, more preferably the groups may be selected from methoxymethyl, ethoxymethyl, methoxyethyl, ethoxyethyl and the like;
the “aryloxyalkyl” group used alone or in combination with other radicals, is selected from phenoxymethyl, napthyloxymethyl, and the like;
the “aralkoxyalkyl” group used alone or in combination with other radicals, is selected from C₆H₅CH₂OCH₂, C₆H₅CH₂OCH₂CH₂, and the like;
the “alkylthio” group used either alone or in combination with other radicals, denotes a straight or branched or cyclic monovalent substituent comprising an allyl group as defined above, linked through a divalent sulfur atom having a free valence bond from the sulfur atom, more preferably the groups may be selected from methylthio, ethylthio, propylthio, butylthio, pentylthio and the like or cyclic alkylthio selected from cyclopropylthio, cyclobutylthio, cyclopentylthio, cyclohexylthio and the like, which may be optionally substituted;
the “thioalkyl” group used either alone or in combination with other radicals, denotes an alkyl group, as defined above, attached to a group of formula —SR′, where R′ represents hydrogen, alkyl or aryl group, e.g. thiomethyl, methylthiomethyl, phenylthiomethyl and the like, which may be optionally substituted.
the “arylthio” group used either alone or in combination with other radicals, is selected from an aryl group, as defined above, linked through a divalent sulfur atom, having a free valence bond from the sulfur atom, more preferably selected from phenylthio, naphthylthio and the like;
the “alkoxycarbonylamino” group used alone or in combination with other radicals, is selected from a suitable alkoxycarbonyl group, as defined above, attached to an amino group, more preferably methoxycarbonylamino, ethoxycarbonylamino, and the like;
the “aryloxycarbonylamino” group used alone or in combination with other radicals, is selected from an aryloxycarbonyl group, as defined above, attached to the an amino group, more preferably such groups are selected from C₆H₅OCONH, C₆H₅OCONCH₃, C₆H₅OCONC₂H₅, C₆H₄(CH₃O)CONH, C₆H₄(OCH₃)OCONH, and the like;
the “aralkoxycarbonylamino” group used alone or in combination with other radicals, is selected from an aralkoxycarbonyl group, as defined above, attached to an amino group, more preferably selected from C₆H₅CH₂OCONH, C₆H₅CH₂CH₂CH₂OCONH, C₆H₅CH₂OCONHCH₃, C₆H₅CH₂OCONC₂H₅, C₆H₄(CH₃)CH₂OCONH, C₆H₄(OCH₃)CH₂OCONH, and the like;
the “aminocarbonylamino”, “alkylaminocarbonylamino”, “dialkylaminocarbonylamino” groups used alone or in combination with other radicals, is a carbonylamino (—CONH₂) group, attached to amino(NH₂), alkylamino group or dialkylamino group respectively, where alkyl group is as defined above;
the “amidino” group used either alone or in combination with other radicals, represents a —C(═N—NH₂ radical; the “alkylamidino” group represents an alkyl radical, as described above, attached to an amidino group;
the “hydrazino” group used either alone or in combination with other radicals, represents a group of the formula —NHNH—, suitably substituted with other radicals, selected from those described above such as an alkyl hydrazino, where an alkyl group, as defined above is attached to a hydrazino group;
the “alkoxyamino” group used either alone or in combination with other radicals, represents a suitable alkoxy group as defined above, attached to an amino group;
the “hydroxyamino” group used either alone or in combination with other radicals, represents a —NHOH moiety, and may be optionally substituted with suitable groups selected from those described above;
the “sulfenyl” group or “sulfenyl derivatives” used alone or in combination with other radicals, represents a bivalent group, —SO— or Rₓ, where Rₓ is an optionally substituted alkyl, aryl, heteroaryl, heterocyclyl, group selected from those described above;
the “sulfonyl” group or “sulfones derivatives” used either alone or in combination with other radicals, with other terms such as alkylsulfonyl, represents a divalent radical —SO₂—, or RₓSO₂—, where Rₓ is as defined above. More preferably, the groups may be selected from “alkylsulfonyl” wherein suitable alkyl radicals, selected from those defined above, is attached to a sulfonyl radical, such as methylsulfonyl, ethylsulfonyl, propylsulfonyl and the like, “arylsulfonyl” wherein an aryl radical, as defined above, is attached to a sulfonyl radical, such as phenylsulfonyl and the like.

Suitable groups and substituents on the groups may be selected from those described anywhere in the specification.

Particularly useful compounds may be selected from
Methyl-2-methyl-5-{4-[2-(5-methyl-2-p-tolyl-oxazol-4-yl)-ethoxy]-benzyl}-[1,3]dioxane-2-carboxylate;
Methyl-5-[6-(2-fluoro-benzyloxy)-naphthalen-2-ylmethyl]-2-methyl-[1,3]dioxane-2-carboxylate;
Methyl-5-[6-(benzyloxy)-naphthalen-2-ylmethyl]-2-methyl-[1,3]dioxane-2-carboxylate;
Methyl-2-methyl-5-cis-{4-[2-(5-methyl-2-phenyl-oxazol-4-yl)-ethoxy]-benzyl}-[1,3]dioxane-2-carboxylate;
Methyl-2-methyl-5-{4-[2-(5-methyl-2-phenyl-oxazol-4-yl)-ethoxy]-benzyl}-[1,3]dioxane-2-carboxylate;
Methyl-2-methyl-5-cis-[4-(5-methyl-2-phenyl-oxazol-4-ylmethoxy)-benzyl]-[1,3]dioxane-2-carboxylate;
Methyl-2-methyl-5-cis-[4-(1-pyridin-2-yl-pyrrolidin-2-ylmethoxy)-benzyl]-[1,3]dioxane-2-carboxylate;
Methyl-2-methyl-5-cis-{4-[2-(methyl-pyridin-2-yl-amino)-ethoxy]-benzyl}-[1,3]dioxane-2-carboxylate;
Methyl-2-methyl-5-[4-(3-methyl-4-oxo-3,4-dihydro-quinazolin-2-ylmethoxy)-benzyl]-[1,3]dioxane-2-carboxylate;
Methyl-2-methyl-5-cis-[4-(5-methyl-2-p-tolyl-oxazol-4-ylmethoxy)-benzyl]-[1,3]dioxane-2-carboxylate;
Methyl-5-cis-[4-(2-carbazol-9-yl-ethoxy)-benzyl]-2-methyl-[1,3]dioxane-2-carboxylate;
Methyl-5-cis-[4-(2-indol-1-yl-ethoxy)-benzyl]-2-methyl-[1,3]dioxane-2-carboxylate;
Methyl-5-{4-[2-(2,3-dihydro-benzo[1,4]oxazin-4-yl)-ethoxy]-benzyl}-2-methyl-[1,3]dioxane-2-carboxylate;
Methyl-2-methyl-5-cis-{4-[5-methyl-2-(5-methyl-thiophen-2-yl)-oxazol-4-ylmethoxy]-benzyl}-[1,3]dioxane-2-carboxylate;
Methyl-2-methyl-5-cis-(4-{2-[5-methyl-2-(5-methyl-thiophen-2-yl)-oxazol-4-yl]-ethoxy}-benzyl)-[1,3]dioxane-2-carboxylate;
Methyl-2-methyl-5-cis-{4-[3-(4-phenoxy-phenoxy)-propoxy]-benzyl}-[1,3]dioxane-2-carboxylate;
Methyl-5-cis-{4-[2-(4-methanesulfonyloxy-phenyl)-ethoxy]-benzyl}-2-methyl-[1,3]dioxane-2-carboxylate;
Methyl-5-cis-[4-(2-tert-Butyl-5-methyl-oxazol-4-ylmethoxy)-benzyl]-2-methyl-[1,3]dioxane-2-carboxylate;
Methyl-2-methyl-5-cis-{4-[4-methyl-2-(4-trifluoromethyl-phenyl)-thiazol-5-ylmethoxy]-benzyl}-[1,3]dioxane-2-carboxylate;
Methyl-2-methyl-5-cis-{4-[2-(4-phenoxy-phenoxy)-ethoxy]-benzyl}-[1,3]dioxane-2-carboxylate;
Methyl-5-cis-[4-(2-fluoro-benzyloxy)-benzyl]-2-methyl-[1,3]dioxane-2-carboxylate;
Methyl-2-methyl-5-[4-(2-phenoxazin-10-yl-ethoxy)-benzyl]-[1,3]dioxane-2-carboxylate;
Methyl-2-methyl-5-cis-[4-(2-oxo-3-phenyl-oxazolidin-5-ylmethoxy)-benzyl]-[1,3]dioxane-2-carboxylate;
Methyl-5-cis-{4-[2-(2,3-dihydro-benzo[1,4]thiazin-4-yl)-ethoxy]-benzyl}-2-methyl-[1,3]dioxane-2-carboxylate;
Methyl-2-methyl-5-cis-[4-(2-phenothiazin-10-yl-ethoxy)-benzyl]-[1,3]dioxane-2-carboxylate;
Methyl-5-cis-{4-[2-(4-hexyl-3-oxo-3,4-dihydro-2H-benzo[1,4]oxazin-2-yl)-ethoxy]-benzyl}-2-methyl-[1,3]dioxane-2-carboxylate,
Methyl-2-methyl-5-cis-(4-{2-[2-methyl-5-(4-methylsulfanyl-phenyl)-pyrrol-1-yl]-ethoxy}-benzyl)-[1,3]dioxane-2-carboxylate;
Methyl-5-cis-{4-[2-(2-tert-Butyl-5-methyl-oxazol-4-yl)-ethoxy]-benzyl}-2-methyl-[1,3]dioxane-2-carboxylate;
(Z)-Methyl-2-methyl-5-cis-{4-[2-(phenyl-pyridin-2-yl-methyleneaminooxy)-ethoxy]-benzyl}-[1,3]dioxane-2-carboxylate;
(E)-Methyl-2-methyl-5-cis-{4-[2-(phenyl-pyridin-2-yl-methyleneaminooxy)-ethoxy]-benzyl}-[1,3]dioxane-2-carboxylate;
Methyl-2-methyl-5-{4-[2-(5-methyl-2-p-tolyl-oxazol-4-yl)-ethoxy]-benzyl}-[1,3]dioxane-2-carboxylate;
Methyl-5-cis-[4-(5-tert-Butyl-2-p-tolyl-2H-pyrazol-3-ylmethoxy)-benzyl]-2-methyl-[1,3]dioxane-2-carboxylate;
Methyl-2-methyl-5-trans-[4-(5-methyl-2-phenyl-oxazol-4-ylmethoxy)-benzyl]-[1,3]dioxane-2-carboxylate;
Methyl-2-methyl-5-trans-[4-(1-pyridin-2-yl-pyrrolidin-2-ylmethoxy)-benzyl]-[1,3]dioxane-2-carboxylate;
Methyl-2-methyl-5-trans-{4-[2-(methyl-pyridin-2-yl-amino)-ethoxy]-benzyl}-[1,3]dioxane-2-carboxylate;
Methyl-2-methyl-5-trans-[4-(5-methyl-2-p-tolyl-oxazol-4-ylmethoxy)-benzyl]-[1,3]dioxane-2-carboxylate;
Methyl-5-trans-[4-(2-carbazol-9-yl-ethoxy)-benzyl]-2-methyl-[1,3]dioxane-2-carboxylate;
Methyl-5-trans-[4-(2-indol-1-yl-ethoxy)-benzyl]-2-methyl-[1,3]dioxane-2-carboxylate;
Methyl-2-methyl-5-trans-{4-[S-methyl-2-(5-methyl-thiophen-2-yl)-oxazol-4-ylmethoxy]-benzyl}-[1,3]dioxane-2-carboxylate;
Methyl-2-methyl-5-trans-(4-{2-[5-methyl-2-(5-methyl-thiophen-2-yl)-oxazol-4-yl]-ethoxy}-benzyl)-[1,3]dioxane-2-carboxylate;
Methyl-2-methyl-5-trans-{4-[3-(4-phenoxy-phenoxy)-propoxy]-benzyl}-[1,3]dioxane-2-carboxylate;
Methyl-5-trans-{4-[2-(4-methanesulfonyloxy-phenyl)-ethoxy]-benzyl}-2-methyl-[1,3]dioxane-2-carboxylate;
Methyl-5-trans-[4-(2-tert-Butyl-5-methyl-oxazol-4-ylmethoxy)-benzyl]-2-methyl-[1,3]dioxane-2-carboxylate;
methyl-2-methyl-5-trans-{4-[4-methyl-2-(4-trifluoromethyl-phenyl)-thiazol-5-ylmethoxy]-benzyl}-[1,3]dioxane-2-carboxylate;
Methyl-2-methyl-5-trans-{4-[2-(4-phenoxy-phenoxy)-ethoxy]-benzyl}-[1,3]dioxane-2-carboxylate;
Methyl-5-trans-[4-(2-fluoro-benzyloxy)-benzyl]-2-methyl-[1,3]dioxane-2-carboxylate;
Methyl-2-methyl-5-trans-[4-(2-oxo-3-phenyl-oxazolidin-5-ylmethoxy)-benzyl]-[1,3]dioxane-2-carboxylate;
Methyl-5-trans-{4-[2-(2,3-dihydro-benzo[1,4]thiazin-4-yl)-ethoxy]-benzyl}-2-methyl-[1,3]dioxane-2-carboxylate;
Methyl-2-methyl-5-trans-[4-(2-phenothiazin-10-yl-ethoxy)-benzyl]-[1,3]dioxane-2-carboxylate;
Methyl-5-trans-{4-[2-(4-hexyl-3-oxo-3,4-dihydro-2H-benzo[1,4]oxazin-2-yl)-ethoxy]benzyl}-2-methyl-[1,3]dioxane-2-carboxylate;
Methyl-2-methyl-5-trans-(4-{2-[2-methyl-5-(4-methylsulfanyl-phenyl)-pyrrol-1-yl]-ethoxy}-benzyl)-[1,3]dioxane-2-carboxylate;
Methyl-5-trans-{4-[2-(2-tert-Butyl-5-methyl-oxazol-4-yl)-ethoxy]-benzyl}-2-methyl-[1,3]dioxane-2-carboxylate;
(Z)-Methyl-2-methyl-5-trans-{4-[2-(phenyl-pyridin-2-yl-methyleneaminooxy)-ethoxy]-benzyl}-[1,3]dioxane-2-carboxylate;
(E)-Methyl-2-methyl-5-trans-{4-[2-(phenyl-pyridin-2-yl-methyleneaminooxy)-ethoxy]-benzyl}-[1,3]dioxane-2-carboxylate;
Methyl-5-trans-[4-(5-tert-Butyl-2-p-tolyl-2H-pyrazol-3-ylmethoxy)-benzyl]-2-methyl-[1,3]dioxane-2-carboxylate;
Methyl-2-methyl-5-cis-[4-(5-methyl-2-phenyl-oxazol-4-ylmethoxy)-phenyl]-[1,3]dioxane-2-carboxylate;
Methyl-2-methyl-5-trans-[4-(5-methyl-2-phenyl-oxazol-4-ylmethoxy)-phenyl]-[1,3]dioxane-2-carboxylate;
Methyl-2-methyl-5-cis-{4-[2-(5-methyl-2-phenyl-oxazol-4-yl)-ethoxy]-phenyl}-[1,3]dioxane-2-carboxylate;
Methyl-2-methyl-5-cis-[4-(2-phenoxazin-10-yl-ethoxy)-phenyl]-[1,3]dioxane-2-carboxylate;
Methyl-2-methyl-5-cis-[4-(3-methyl-4-oxo-3,4-dihydro-quinazolin-2-ylmethoxy)-phenyl]-[1,3]dioxane-2-carboxylate;
Methyl-5-cis-[4-(2-indol-1-yl-ethoxy)-phenyl]-2-methyl-[1,3]dioxane-2-carboxylate;
Methyl-5-cis-{4-[2-(5-ethyl-pyridin-2-yl)-ethoxy]-phenyl}-2-methyl-[1,3]dioxane-2-carboxylate;
Methyl-2-methyl-5-cis-[4-(5-methyl-2-p-tolyl-oxazol-4-ylmethoxy)-phenyl]-[1,3]dioxane-2-carboxylate;
Methyl-2-Methyl-5-cis-{4-[2-(5-methyl-2-p-tolyl-oxazol-4-yl)-ethoxy]-phenyl}-[1,3]dioxane-2-carboxylate;
Methyl-5-cis-{4-[2-(4-methanesulfonyloxy-phenyl)-ethoxy]-phenyl}-2-methyl-[1,3]dioxane-2-carboxylate;
Methyl-2-methyl-5-trans-[4-(5-methyl-2-p-tolyl-oxazol-4-ylmethoxy)-phenyl]-[1,3]dioxane-2-carboxylate;
Methyl-2-methyl-5-trans-{4-[2-(5-methyl-2-phenyl-oxazol-4-yl)-ethoxy]-phenyl}-[1,3]dioxane-2-carboxylate;
Methyl-2-methyl-5-trans-{4-[2-(5-methyl-2-p-tolyl-oxazol-4-yl)-ethoxy]-phenyl}-[1,3]dioxane-2-carboxylate;
Methyl-2-methyl-5-trans-{4-[4-methyl-2-(4-trifluoromethyl-phenyl)-thiazol-5-ylmethoxy]-phenyl}-[1,3]dioxane-2-carboxylate;
Methyl-2-methyl-5-cis-{4-[4-methyl-2-(4-trifluoromethyl-phenyl)-thiazol-5-ylmethoxy]-phenyl}-[1,3]dioxane-2-carboxylate;
Methyl-2-methyl-5-cis-{4-(2-[2-methyl-5-(4-methylsulfanyl-phenyl)-pyrrol-1-yl]-ethoxy}-phenyl)-[1,3]dioxane-2-carboxylate;
Methyl-5-cis-[4-(2-tert-Butyl-5-methyl-oxazol-4-ylmethoxy)-phenyl]-2-methyl-[1,3]dioxane-2-carboxylate;
Methyl-5-trans-[4-(2-tert-Butyl-5-methyl-oxazol-4-ylmethoxy)-phenyl]-2-methyl-[1,3]dioxane-2-carboxylate;
Methyl-2-methyl-5-cis-{4-[2-(4-phenoxy-phenoxy)-ethoxy]-phenyl}-[1,3]dioxane-2-carboxylate;
Methyl-2-methyl-5-trans-{4-[2-(4-phenoxy-phenoxy)-ethoxy]-phenyl}-[1,3]dioxane-2-carboxylate;
Methyl-5-cis-[4-(2-fluoro-benzyloxy)-phenyl]-2-methyl-[1,3]dioxane-2-carboxylate;
Methyl-5-trans-[4-(2-fluoro-benzyloxy)-phenyl]-2-methyl-[1,3]dioxane-2-carboxylate;
Methyl-5-cis-[4-(5-tert-Butyl-2-p-tolyl-2H-pyrazol-3-ylmethoxy)-phenyl]-2-methyl-[1,3]dioxane-2-carboxylate;
Methyl-5-trans-[4-(5-tert-Butyl-2-p-tolyl-2H-pyrazol-3-ylmethoxy)-phenyl]-2-methyl-[1,3]dioxane-2-carboxylate;
Methyl-2-methyl-5-trans-[4-(2-oxo-3-phenyl-oxazolidin-5-ylmethoxy)-phenyl]-[1,3]dioxane-2-carboxylate;
Methyl-2-methyl-5-cis-[4-(2-oxo-3-phenyl-oxazolidin-5-ylmethoxy)-phenyl]-[1,3]dioxane-2-carboxylate;
Methyl-5-trans-{4-[2-(4-Methanesulfonyloxy-phenyl)-ethoxy]-phenyl}-2-methyl-[1,3]dioxane-2-carboxylate;
Methyl-{2-Methyl-5-trans-[4-(3-methyl-4-oxo-3,4-dihydro-quinazolin-2-ylmethoxy)-phenyl]-[1,3]dioxane-2 carboxylate;
2-methyl-5-{4-[2-(5-methyl-2-phenyl-oxazol-4-yl)-ethoxy]-benzyl}-[1,3]dioxane-2-carboxylic acid and its pharmaceutically acceptable salts;
2-Methyl-5-{4-[2-(5-methyl-2-p-tolyl-oxazol-4-yl)-ethoxy]-benzyl}-[1,3]dioxane-2-carboxylic acid and its pharmaceutically acceptable salts;
2-Methyl-5-cis-[4-(5-methyl-2-phenyl-oxazol-4-ylmethoxy)-benzyl]-[1,3]dioxane-2-carboxylic acid and its pharmaceutically acceptable salts;
2-Methyl-5-cis-[4-(1-pyridin-2-yl-pyrrolidin-2-ylmethoxy)-benzyl]-[1,3]dioxane-2-carboxylic acid and its pharmaceutically acceptable salts;
2-Methyl-5-cis-{4-[2-(methyl-pyridin-2-yl-amino)-ethoxy]-benzyl}-[1,3]dioxane-2-carboxylic acid and its pharmaceutically acceptable salts;
2-Methyl-5-[4-(3-methyl-4-oxo-3,4-dihydro-quinazolin-2-ylmethoxy)-benzyl]-[1,3]dioxane-2-carboxylic acid and its pharmaceutically acceptable salts;
2-Methyl-5-[4-(2-phenoxazin-10-yl-ethoxy)-benzyl]-[1,3]dioxane-2-carboxylic acid and its pharmaceutically acceptable salts;
2-Methyl-5-cis-[4-(5-methyl-2-p-tolyl-oxazol-4-ylmethoxy)-benzyl]-[1,3]dioxane-2-carboxylic acid and its pharmaceutically acceptable salts;
5-cis-[4-(2-Carbazol-9-yl-ethoxy)-benzyl]-2-methyl-[1,3]dioxane-2-carboxylic acid and its pharmaceutically acceptable salts;
5-cis-[4-(2-Indol-1-yl-ethoxy)-benzyl]-2-methyl-[1,3]dioxane-2-carboxylic acid and its pharmaceutically acceptable salts;
5-{4-[2-(2,3-Dihydro-benzo[1,4]oxazin-4-yl)-ethoxy]-benzyl}-2-methyl-[1,3]dioxane-2-carboxylic acid and its pharmaceutically acceptable salts;
2-Methyl-5-cis-{4-[5-methyl-2-(5-methyl-thiophen-2-yl)-oxazol-4-ylmethoxy]-benzyl}-[1,3]dioxane-2-carboxylic acid and its pharmaceutically acceptable salts;
5-cis-{4-[2-(2,3-Dihydro-benzo[1,4]thiazin-4-yl)-ethoxy]-benzyl}-2-methyl-[1,3]dioxane-2-carboxylic acid and its pharmaceutically acceptable salts;
2-Methyl-5-cis-[4-(2-phenothiazin-10-yl-ethoxy)-benzyl]-[1,3]dioxane-2-carboxylic acid and its pharmaceutically acceptable salts;
5-cis-{4-[2-(4-Hexyl-3-oxo-3,4-dihydro-2H-benzo[1,4]oxazin-2-yl)-ethoxy]-benzyl}-2-methyl-[1,3]dioxane-2-carboxylic acid and its pharmaceutically acceptable salts;
2-Methyl-5-cis-(4-{2-[5-methyl-2-(5-methyl-thiophen-2-yl)-oxazol-4-yl]-ethoxy}-benzyl)-[1,3]dioxane-2-carboxylic acid and its pharmaceutically acceptable salts;
2-Methyl-5-cis-{4-[2-(5-methyl-2-phenyl-oxazol-4-yl)-ethoxy]-benzyl}-[1,3]dioxane-2-carboxylic acid and its pharmaceutically acceptable salts;
2-Methyl-5-trans-{4-[2-(5-methyl-2-phenyl-oxazol-4-yl)-ethoxy]-benzyl}-[1,3]dioxane-2-carboxylic acid and its pharmaceutically acceptable salts;
2-Methyl-5-cis-{4-[3-(4-phenoxy-phenoxy)-propoxy]-benzyl}-[1,3]dioxane-2-carboxylic acid and its pharmaceutically acceptable salts;
5-cis-{4-[2-(4-Methanesulfonyloxy-phenyl)-ethoxy]-benzyl}-2-methyl-[1,3]dioxane-2-carboxylic acid and its pharmaceutically acceptable salts;
5-cis-[4-(2-tert-Butyl-5-methyl-oxazol-4-ylmethoxy)-benzyl]-2-methyl-[1,3]dioxane-2-carboxylic acid and its pharmaceutically acceptable salts;
2-Methyl-5-cis-{4-[4-methyl-2-(4-trifluoromethyl-phenyl)-thiazol-5-ylmethoxy]-benzyl}-[1,3]dioxane-2-carboxylic acid and its pharmaceutically acceptable salts;
2-Methyl-5-cis-{4-[2-(4-phenoxy-phenoxy)-ethoxy]-benzyl}-[1,3]dioxane-2-carboxylic acid and its pharmaceutically acceptable salts;
5-cis-[4-(2-Fluoro-benzyloxy)-benzyl]-2-methyl-[1,3]dioxane-2-carboxylic acid and its pharmaceutically acceptable salts;
2-Methyl-5-cis-(4-{2-[2-methyl-5-(4-methylsulfanyl-phenyl)-pyrrol-1-yl]-ethoxy}-benzyl)-[1,3]dioxane-2-carboxylic acid and its pharmaceutically acceptable salts;
5-cis-{4-[2-(2-tert-Butyl-5-methyl-oxazol-4-yl)-ethoxy]-benzyl}-2-methyl-[1,3]dioxane-2-carboxylic acid and its pharmaceutically acceptable salts;
5-cis-[6-(2-Fluoro-benzyloxy)-naphthalen-2-ylmethyl]-2-methyl-[1,3]dioxane-2-carboxylic acid and its pharmaceutically acceptable salts;
5-cis-[6-(Benzyloxy)-naphthalen-2-ylmethyl]-2-methyl-[1,3]dioxane-2-carboxylic acid and its pharmaceutically acceptable salts;
(Z)-2-Methyl-5-cis-{4-[2-(phenyl-pyridin-2-yl-methyleneaminooxy)-ethoxy]-benzyl}-[1,3]dioxane-2-carboxylic acid and its pharmaceutically acceptable salts;
(E)-2-Methyl-5-cis-{4-[2-(phenyl-pyridin-2-yl-methyleneaminooxy)-ethoxy]-benzyl}-[1,3]dioxane-2-carboxylic acid and its pharmaceutically acceptable salts;
5-cis-[4-(5-tert-Butyl-2-p-tolyl-2H-pyrazol-3-ylmethoxy)-benzyl]-2-methyl-[1,3]dioxane-2-carboxylic acid and its pharmaceutically acceptable salts;
2-Methyl-5-cis-{4-[2-(5-methyl-2-p-tolyl-oxazol-4-yl)-ethoxy]-benzyl}-[1,3]dioxane-2-carboxylic acid and its pharmaceutically acceptable salts;
2-Methyl-5-trans-[4-(5-methyl-2-phenyl-oxazol-4-ylmethoxy)-benzyl]-[1,3]dioxane-2-carboxylic acid and its pharmaceutically acceptable salts;
2-Methyl-5-trans-[4-(1-pyridin-2-yl-pyrrolidin-2-ylmethoxy)-benzyl]-[1,3]dioxane-2-carboxylic acid and its pharmaceutically acceptable salts;
2-Methyl-5-trans-{4-[2-(methyl-pyridin-2-yl-amino)-ethoxy]-benzyl}-[1,3]dioxane-2-carboxylic acid and its pharmaceutically acceptable salts;
2-Methyl-5-trans-[4-(5-methyl-2-p-tolyl-oxazol-4-ylmethoxy)-benzyl]-[1,3]dioxane-2-carboxylic acid and its pharmaceutically acceptable salts;
5-trans-[4-(2-Carbazol-9-yl-ethoxy)-benzyl]-2-methyl-[1,3]dioxane-2-carboxylic acid and its pharmaceutically acceptable salts;
5-trans-[4-(2-Indol-1-yl-ethoxy)-benzyl]-2-methyl-[1,3]dioxane-2-carboxylic acid and its pharmaceutically acceptable salts;
2-Methyl-5-trans-{4-[5-methyl-2-(5-methyl-thiophen-2-yl)-oxazol-4-ylmethoxy]-benzyl}-[1,3]dioxane-2-carboxylic acid and its pharmaceutically acceptable salts;
5-trans-{4-[2-(2,3-Dihydro-benzo[1,4]thiazin-4-yl)-ethoxy]-benzyl}-2-methyl-[1,3]dioxane-2-carboxylic acid and its pharmaceutically acceptable salts;
2-Methyl-5-trans-[4-(2-phenothiazin-10-yl-ethoxy)-benzyl]-[1,3]dioxane-2-carboxylic acid and its pharmaceutically acceptable salts;
5-trans-{4-[2-(4-Hexyl-3-oxo-3,4-dihydro-2H-benzo[1,4]oxazin-2-yl)-ethoxy]-benzyl}-2-methyl-[1,3]dioxane-2-carboxylic acid and its pharmaceutically acceptable salts;
2-Methyl-5-trans-(4-{2-[5-methyl-2-(5-methyl-thiophen-2-yl)-oxazol-4-yl]-ethoxy}-benzyl)-[1,3]dioxane-2-carboxylic acid and its pharmaceutically acceptable salts;
2-Methyl-5-trans-{4-[2-(5-methyl-2-phenyl-oxazol-4-yl)-ethoxy]-benzyl}-[1,3]dioxane-2-carboxylic acid and its pharmaceutically acceptable salts;
2-Methyl-5-trans-{4-[2-(5-methyl-2-phenyl-oxazol-4-yl)-ethoxy]-benzyl}-[1,3]dioxane-2-carboxylic acid and its pharmaceutically acceptable salts;
2-Methyl-5-trans-{4-[3-(4-phenoxy-phenoxy)-propoxy]-benzyl}-[1,3]dioxane-2-carboxylic acid and its pharmaceutically acceptable salts;
5-trans-{4-[2-(4-Methanesulfonyloxy-phenyl)-ethoxy]-benzyl}-2-methyl-[1,3]dioxane-2-carboxylic acid and its pharmaceutically acceptable salts;
5-trans-[4-(2-tert-Butyl-5-methyl-oxazol-4-ylmethoxy)-benzyl]-2-methyl-[1,3]dioxane-2-carboxylic acid and its pharmaceutically acceptable salts;
2-Methyl-5-trans-{4-[4-methyl-2-(4-trifluoromethyl-phenyl)-thiazol-5-ylmethoxy]-benzyl}-[1,3]dioxane-2-carboxylic acid and its pharmaceutically acceptable salts;
2-Methyl-5-trans-{4-[2-(4-phenoxy-phenoxy)-ethoxy]-benzyl}-[1,3]dioxane-2-carboxylic acid and its pharmaceutically acceptable salts;
5-trans-[4-(2-Fluoro-benzyloxy)-benzyl]-2-methyl-[1,3]dioxane-2-carboxylic acid and its pharmaceutically acceptable salts;
2-Methyl-5-trans-(4-{2-[2-methyl-5-(4-methylsulfanyl-phenyl)-pyrrol-1-yl]-ethoxy}-benzyl)-[1,3]dioxane-2-carboxylic acid and its pharmaceutically acceptable salts;
5-trans-{4-[2-(2-tert-Butyl-5-methyl-oxazol-4-yl)-ethoxy]-benzyl}-2-methyl-[1,3]dioxane-2-carboxylic acid and its pharmaceutically acceptable salts;
5-trans-[6-(2-Fluoro-benzyloxy)-naphthalen-2-ylmethyl]-2-methyl-[1,3]dioxane-2-carboxylic acid and its pharmaceutically acceptable salts;
5-trans-[6-(Benzyloxy)-naphthalen-2-ylmethyl]-2-methyl-[1,3]dioxane-2-carboxylic acid and its pharmaceutically acceptable salts;
(Z)-2-Methyl-5-trans-{4-[2-(phenyl-pyridin-2-yl-methyleneaminooxy)-ethoxy]-benzyl}-[1,3]dioxane-2-carboxylic acid and its pharmaceutically acceptable salts;
(E)-2-Methyl-5-trans-{4-[2-(phenyl-pyridin-2-yl-methyleneaminooxy)-ethoxy]-benzyl}-[1,3]dioxane-2-carboxylic acid and its pharmaceutically acceptable salts;
5-trans-[4-(5-tert-Butyl-2-p-tolyl-2H-pyrazol-3-ylmethoxy)-benzyl]-2-methyl-[1,3]dioxane-2-carboxylic acid and its pharmaceutically acceptable salts;
2-Methyl-5-trans-{4-[2-(5-methyl-2-p-tolyl-oxazol-4-yl)-ethoxy]-benzyl}-[1,3]dioxane-2-carboxylic acid and its pharmaceutically acceptable salts;
2-Methyl-5-cis-[4-(5-methyl-2-phenyl-oxazol-4-ylmethoxy)-phenyl]-[1,3]dioxane-2-carboxylic acid and its pharmaceutically acceptable salts;
2-Methyl-5-cis{4-[2-(5-methyl-2-phenyl-oxazol-4-yl)-ethoxy]-phenyl}-[1,3]dioxane-2-carboxylic acid and its pharmaceutically acceptable salts;
2-Methyl-5-cis-[4-(2-phenoxazin-10-yl-ethoxy)-phenyl]-[1,3]dioxane-2-carboxylic acid and its pharmaceutically acceptable salts;
2-Methyl-5-cis-[4-(3-methyl-4-oxo-3,4-dihydro-quinazolin-2-ylmethoxy)-phenyl]-[1,3]dioxane-2-Carboxylic acid and its pharmaceutically acceptable salts;
5-cis-[4-(2-Indol-1-yl-ethoxy)-phenyl]-2-methyl-[1,3]dioxane-2-carboxylic acid and its pharmaceutically acceptable salts;
5-cis-{4-[2-(5-Ethyl-pyridin-2-yl)-ethoxy]-phenyl}-2-methyl-[1,3]dioxane-2-carboxylic acid and its pharmaceutically acceptable salts;
2-Methyl-5-cis-[4-(5-methyl-2-p-tolyl-oxazol-4-ylmethoxy)-phenyl]-[1,3]dioxane-2-carboxylic acid and its pharmaceutically acceptable salts;
2-Methyl-5-cis-{4-[2-(5-methyl-2-p-tolyl-oxazol-4-yl)-ethoxy]-phenyl}-[1,3]dioxane-2-carboxylic acid and its pharmaceutically acceptable salts;
5-cis-{4-[2-(4-Methanesulfonyloxy-phenyl)-ethoxy]-phenyl}-2-methyl-[1,3]dioxane-2-carboxylic acid and its pharmaceutically acceptable salts;
2-Methyl-5-trans-[4-(5-methyl-2-phenyl-oxazol-4-ylmethoxy)-phenyl]-[1,3]dioxane-2-carboxylic acid and its pharmaceutically acceptable salts;
2-Methyl-5-trans-[4-(5-methyl-2-p-tolyl-oxazol-4-ylmethoxy)-phenyl]-[1,3]dioxane-2-carboxylic acid and its pharmaceutically acceptable salts;
2-Methyl-5-trans-{4-[2-(5-methyl-2-phenyl-oxazol-4-yl)-ethoxy]-phenyl}-[1,3]dioxane-2-carboxylic acid and its pharmaceutically acceptable salts;
2-Methyl-5-trans-{4-[2-(5-methyl-2-p-tolyl-oxazol-4-yl)-ethoxy]-phenyl}-[1,3]dioxane-2-carboxylic acid and its pharmaceutically acceptable salts;
2-Methyl-5-trans-{4-[4-methyl-2-(4-trifluoromethyl-phenyl)-thiazol-5-ylmethoxy]-phenyl}-[1,3]dioxane-2-carboxylic acid and its pharmaceutically acceptable salts;
2-Methyl-5-cis-{4-[4-methyl-2-(4-trifluoromethyl-phenyl)-thiazol-5-ylmethoxy]-phenyl}-[1,3]dioxane-2-carboxylic acid and its pharmaceutically acceptable salts;
2-Methyl-5-cis-(4-{2-[2-methyl-5-(4-methylsulfanyl-phenyl)-pyrrol-1-yl]-ethoxy}-phenyl)-[1,3]dioxane-2-carboxylic acid and its pharmaceutically acceptable salts;
5-cis-[4-(2-tert-Butyl-5-methyl-oxazol-4-ylmethoxy)-phenyl]-2-methyl-[1,3]dioxane-2-carboxylic acid and its pharmaceutically acceptable salts;
5-trans-[4-(2-tert-Butyl-5-methyl-oxazol-4-ylmethoxy)-phenyl]-2-methyl-[1,3]dioxane-2-carboxylic acid and its pharmaceutically acceptable salts;
2-Methyl-5-cis-{4-[2-(4-phenoxy-phenoxy)-ethoxy]-phenyl}-[1,3]dioxane-2-carboxylic acid and its pharmaceutically acceptable salts;
2-Methyl-5-trans-{4-[2-(4-phenoxy-phenoxy)-ethoxy]-phenyl}-[1,3]dioxane-2-carboxylic acid and its pharmaceutically acceptable salts;
5-cis-[4-(2-Fluoro-benzyloxy)-phenyl]-2-methyl-[1,3]dioxane-2-carboxylic acid and its pharmaceutically acceptable salts;
5-trans-[4-(2-Fluoro-benzyloxy)-phenyl]-2-m yl-[1,3]dioxane-2-carboxylic acid and its pharmaceutically acceptable salts;
5-cis-[4-(5-tert-Butyl-2-p-tolyl-2H-pyrazol-3-ylmethyl)-phenyl]-2-methyl-[1,3]dioxane-2-carboxylic acid and its pharmaceutically acceptable salts;
5-trans-[4-(5-tert-Butyl-2-p-tolyl-2H-pyrazol-3-ylmethoxy)-phenyl]-2-methyl-[1,3]dioxane-2-carboxylic acid and its pharmaceutically acceptable salts;
2-Methyl-5-trans-[4-(2-oxo-3-phenyl-oxazolidin-5-ylmethoxy)-phenyl]-[1,3]dioxane-2-carboxylic acid and its pharmaceutically acceptable salts;
2-Methyl-5-cis-[4-(2-oxo-3-phenyl-oxazolidin-5-ylmethoxy)-phenyl]-[1,3]dioxane-2-carboxylic acid and its pharmaceutically acceptable salts;
5-trans-{4-[2-(4-Methanesulfonyloxy-phenyl}-ethoxy]-phenyl-2-methyl-[1,3]dioxane-2-carboxylic acid and its pharmaceutically acceptable salts;
2-Methyl-5-trans-[4-(3-methyl-4-oxo-3,4-dihydro-quinazolin-2-ylmethoxy)-phenyl]-[1,3]dioxane-2-carboxylic acid and its pharmaceutically acceptable salts;

The novel compounds of this invention may be prepared using the reactions and techniques described in this section. The reactions are performed in solvents appropriate to the reagents and materials employed and are suitable for the transformations being effected. It is understood by those skilled in the art that the nature and order of the synthetic steps presented may be varied for the purpose of optimizing the formation of the compounds of the present invention.

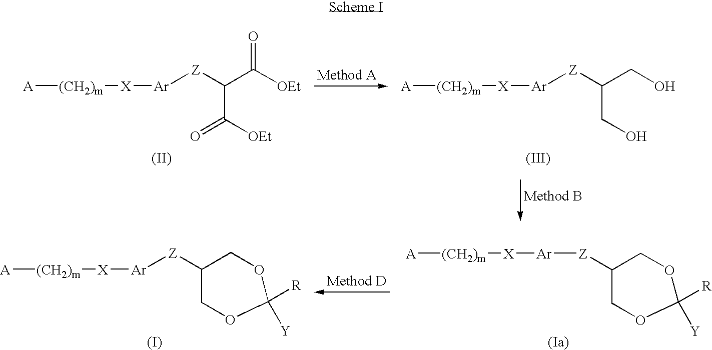

i. reduction of compound of formula (II) wherein all the symbols are as defined earlier to compound of formula (III) wherein all the symbols are as defined earlier.

ii. reacting compound of formula (III) wherein all the symbols are as defined earlier with suitable ketoester of the formula RC(O)Y wherein R is as defined earlier and Y is COOR¹ where R¹ is alkyl or aryl to yield compound of formula (Ia) wherein Y represents COOR¹ where R¹ is alkyl or aryl and all other symbols are as defined earlier.

iii. hydrolysis of compound of general formula (Ia) wherein Y is COOR¹ where R¹ is alkyl or aryl and all other symbols are as defined earlier to yield compound of general formula (I) wherein Y is COOH and all other symbols are as defined earlier.

iv. compound of formula (I) where Y represents COOH or (Ia) where Y represents COOR¹ where R¹ represents alkyl or aryl and all other symbols are as defined earlier may optionally be converted to further compound of formula (I) where Y represents CONR¹R² where in all the symbols are as defined earlier by reacting with appropriate amine. The reactions can be carried out by suitable modifications of methods & techniques known to those skilled in the art. As an example of general techniques and methods which may be used, the techniques described in “Comprehensive Organic Transformations” R. C. Larock (2^{nd} Ed., 1999) (VCH Publishers Inc.) and “Advanced Organic Chemistry”, J. March (4^{th} Ed.), John Wiley & Sons may be used with appropriate modifications.

Method A: The diester of the formula (II) may be reduced to diol of formula (III). Suitable reducing agents may be hydrides such as LiAlH₄, NaBH₄, diborane, NaBH₄/BF₃OEt₂, LiBH₄, DIBAH, and the like. Reaction may be carried out in suitable solvents appropriate for the reducing agent used e.g. with LiAlH₄, NaBH₃, diborane, NaBH₄/BF₃OEt₂ aprotic solvents such as THF, ether and the likes or their combinations are preferred. With NaBH₄, LiBH₄ etc. alcoholic solvents used alone or as mixtures may also be used. The reaction may be carried out at a temperature in the range 0° C. to reflux temperature of the solvent(s) used and the reaction time may range from 1 to 24 hours.

Method D: The diol of formula (I) may be converted to dioxane of formula (Ia) by reacting with appropriate ketoester (RC(O)COOR¹) in presence of a Lewis acid e.g. boron trifluoride etherate complex and the like. Reaction may be conducted in an appropriate solvent e.g., polar solvent such as acetonitrile or N,N-dimethyl formamide (DMF), ether solvent such as tetrahydrofuran (THF) or diethyl ether, diisopropyl ether, 1,2-dimethoxyethane, halogenated hydrocarbon solvents such as chloroform or dichloromethane, hydrocarbon solvent such as benzene, toluene, hexane, heptane or a mixtures of appropriate solvents selected from those described above. The reaction may be carried out at a temperature in the range −20° C. to reflux temperature of the solvent(s) used and the reaction time may range from 1 to 48 hours.

Method D: The compound of formula (Ia) may be hydrolysed to compound of formula (I) using suitable base e.g., NaOH, LiOH, KOH and the like. Reaction may be conducted in suitable solvents e.g., alcohols like methanol, ethanol, propanol, isopropanol, butanol and the like, THF, water or mixtures thereof. The reaction may be carried out at a temperature in the range 20° C. to reflux temperature of the solvent(s) used and the reaction time may range from 1 to 48 hours.

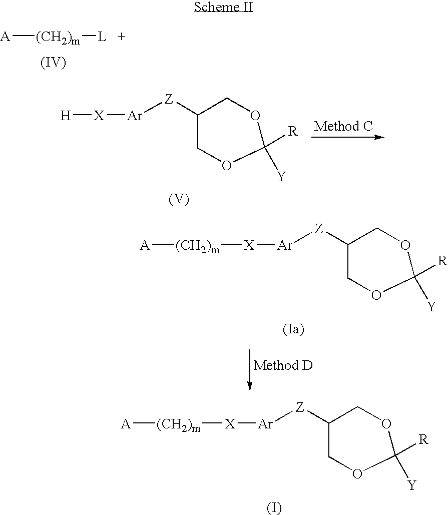

i. reacting compounds of general formula (IV) where all symbols are as defined earlier and L represents a leaving group such as halogen, mesylate, tosylate, triflate & the like with compounds of general formula (V), where all symbols are as defined earlier and Y represent COOR¹ where R¹ represents alkyl or aryl to yield compound of general formula (Ia) where all symbols are as defined earlier and Y represent COOR¹ where R¹ represents alkyl or aryl.

ii. hydrolysis of compound of general formula (Ia) wherein Y is COOR¹ where R¹ represents alkyl or aryl and all other symbols are as defined earlier to yield compound of general formula (I) where in Y is COOH and all other symbols are as defined earlier.

Method C: The compound of formula (Ia) may be prepared by reacting compound of formula (IV) with compound of formula (V) under suitable conditions. The reaction may be carried out in presence of solvents such as acetone, tetrahydrofuran, dimethyl sulfoxide, dioxane, acetonitrile, dimethyl formamide, benzene, toluene, petroleum ether, heptane, hexane, 2-butanone, xylene, alcohols such as methanol, ethanol, propanol, butanol, iso-butanol tertbutanol, pentanol and the like or mixtures of appropriate solvents selected from those above. Bases such as alkali metal carbonates such as K₂CO₃, Na₂CO₃, CsCO₃, and the like; or alkali metal hydroxides such as NaOH, KOH and the like, may be used in this reaction. Alkali metal hydrides such as NaH, KH can be used whenever solvent employed is not protic or contain carbonyl group. The reaction may be carried out at a temperature in the range 0° C. to reflux temperature of the solvent(s) used and the reaction time may range from 1 to 48 hours. The intermediate of general formula (V) may be prepared by one or more routes or combinations of reactions outlined in scheme III outlined below which comprises:

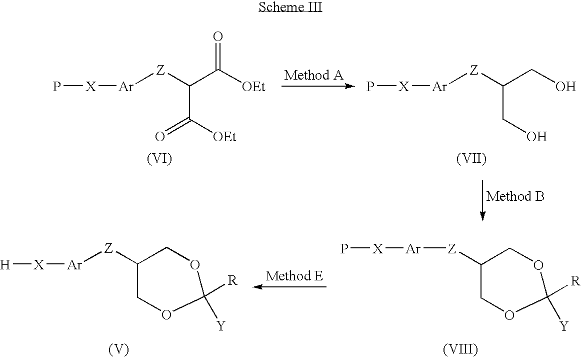

i. reduction of compound of formula (VI) to compound of formula (VII) wherein all the symbols are as defined earlier and P represents a suitable protecting group for e.g. benzyl, methoxymethyl and the like.

ii. reacting compound of formula (V with suitable ketoester of the formula RC(O)Y wherein R is as defined earlier and Y represents COOR¹ where R¹ is alkyl or aryl to yield compound of formula (VIII) wherein P represents a suitable protecting group i.e benzyl, methoxymethyl and the like and Y represents COOR¹ where R¹ is alkyl or aryl and all other symbols are as defined earlier.

iii. reprotection of compound of formula (VIII) to yield compound of formula (V) wherein Y represents COOR¹ where R¹ represents alkyl or aryl and all other symbols are as defined earlier.

Method A: The compound of formula (VI) may be reduced to compound of formula (VII) by a suitable reducing agent as described in method A earlier.

Method B: The diol of formula (VI) may be converted to a compound of formula (VIII) by a procedure similar to that described in method B earlier.

method E: The compound of formula (VIII) may be deprotected to yield compound of formula (V). Suitable deprotecting methods known in the art for e.g. in T. W. Greene and P. G. M. Wuts “Protective groups in Organic Synthesis”, John Wiley L, Sons, Inc, 1999, 3^{rd} Ed., 201-245 along with references therein may be employed depending on the protecting group used.

The invention is explained in greater detail by the examples given below, which are provided by way of illustration only and therefore should not be construed to limit the scope of the invention.

It will be appreciated that one or more of the processes described in the general schemes above may be used to prepare the compounds of the present invention.

1H spectral data given in the tables (vide infra) are recorded using a 300 MHz spectrometer (Bruker A VANCE-300) and reported in δ scale. Until and otherwise mentioned the solvent used for NMR is CDCl₃ using tetramethyl silane as the internal standard.

### Example 1

### Methyl-2-methyl-5-{4-[2-(5-methyl-2-p-tolyl-oxazol-4-yl)-ethoxy]-benzyl}-[1,3]dioxane-2-carboxylate

To a solution of 2-{4-[2-(5-Methyl-2-p-tolyl-oxazol-4-yl)-ethoxy]-benzyl}-propane-1,3-diol (1 g) in acetonitrile (10 mL) was added methyl pyruvate (0.94 mL) followed by 98% boron trifluoride diethyl ether complex (0.65 mL) and the reaction mixture was stirred at ambient temperature for extended hours (tlc). The reaction mixture was poured in to a solution of sodium bicarbonate and extracted with ethyl acetate. The combined organic extract was washed with water, brine solution, dried over sodium sulphate and evaporated under reduced pressure. The crude product was flash chromatographed over silicagel using a mixture of ethyl acetate and petroleum ether as eluent to obtain 910 mg of pure product.

¹H NMR: 1.59 (3H, s), 2.26 (2H, s), 2.35 (3H, s), 2.38 (3H, s), 2.91-2.97 (3H, m), 3.45 (2H, t, J=10.9 Hz), 3.7-3.9 (5H, m), 4.2 (2H, t, J=6.7 Hz), 6.82 (2H, t) J=7.2 Hz), 6.97 (1H, d, J=8.46 Hz), 7.09 (1H, t, J=8.48 Hz), 7.23 (2H, d, J=8.07 Hz), 7.85 (2H, d, J=8.07 Hz).

Yield: 74%

### Example 2

### Methyl-2-methyl-5-cis-{4-[2-(5-methyl-2-phenyl-oxazol-4-yl)-ethoxy]-benzyl}-[1,3]dioxane-2-carboxylate

### Step 1: Preparation of Methyl-5-(4-benzyloxy-benzyl)-2-methyl-[1,3]dioxane-2-carboxylate

2-(4-Benzyloxy-benzyl)-propane-1,3-diol (37 g) was dissolved in 200 mL of acetonitrile, and 50.3 mL of methyl pyruvate was added. To the mixture, 39.2 mL of boron trifluoride diethyl ether complex (98%) was added with stirring at ambient temperature, and stirring was continued for 3-6 hours at ambient temperature. The reaction mixture was poured into an aqueous solution of sodium bicarbonate and extracted with ethyl acetate. The organic extract was washed with water, dried over sodium sulfate and evaporated under reduced pressure. The crude product was flash chromatographed over silica gel using a mixture of ethyl acetate and petroleum ether as eluent to obtain 18 g of pure product.

### Step 2. Preparation of cis Methyl-5-(4-hydroxy-benzyl)-2-methyl-[1,3]dioxane-2-carboxylate

To a suspension of 10% palladium on charcoal (3.4 g) in methanol (100 mL) was added Methyl-5-(4-benzyloxy-benzyl)-2-methyl-[1,3]dioxane-2-carboxylate (18 g) prepared in step 1 above followed by ammonium formate (13 g) and the reaction mixture was heated to reflux for 2-5 hours. The reaction mixture was cooled to ambient temperature and the catalyst was filtered off. The filtrate was evaporated, the residue was taken in ethyl acetate and washed with water. The organic extract was dried over sodium sulfate and evaporated under reduced pressure to yield 13 g of product. This was re-crystallised from a mixture of ethyl acetate and petroleum ether to obtain 7 g of the desired product.

### Step 3: Preparation of cis Methyl-2-methyl-5-{4-[2-(5-methyl-2-phenyl-oxazol-4-yl)-ethoxy]-benzyl}-[1,3]dioxane-2-carboxylate

A mixture of cis methyl-5-(4-hydroxy-benzyl)-2-methyl-[1,3]dioxane-2-carboxylate (prepared in step 2 above) (750 mg) 2-(5-methyl-2-phenyl-oxazol-4-yl)-ethyl methane sulfonate (790 mg) and potassium carbonate (780 mg) in anhydrous dimethyl formamide (10 mL) was stirred at 80° C. for extended periods in an inert atmosphere. The reaction mixture was cooled to ambient temperature, poured into ice cold water and extracted with ethyl acetate. The combined organic extract was washed with water, brine solution, dried over sodium sulphate and evaporated under reduced pressure. The crude product was flash chromatographed over silica gel using a mixture of ethyl acetate and petroleum ether as eluent to obtain 971 mg of pure product.

¹H NMR: 1.49 (3H, s), 2.27 (3H, s), 2.32 (3H, s), 2.96 (2H, t, J=6.66 Hz), 3.45 (2H, t, J=10.4 Hz), 3.83-3.9 (5H, m), 4.21 (2H, t, J=6.72 Hz), 6.73-6.75 (3H, dd, J=6.57 & 2.01 Hz), 6.97 (3H, dd, J=3.55 & 6.57 Hz), 7.39-7.44 (2H, m), 7.97 (1H, dd, J=7.92 & 2.46 Hz).

Yield: 76%

The following compounds are prepared by procedure similar to those described in examples 1 or 2 with appropriate variations of reactants, reaction conditions and quantities of reagents.

### Example 3

### Methyl-2-methyl-5-{4-[2-(5-methyl-2-phenyl-oxazol-4-yl)-ethoxy]-benzyl}-[1,3]dioxane-2-carboxylate

¹H NMR: 1.49 (3H, s), 2.26 (3H, s), 2.37 (3H, s), 2.91-2.99 (3H, q, J=13.74 & 6.69 Hz), 3.45 (2H, t, J=10.44 Hz), 3.73-3.93 (5H, m), 4.21 (2H, t, J=6.72 Hz), 6.82 (3H, t, J=7.23 Hz), 6.98 (1H, d, J=8.55 Hz), 7.10 (1H, d, J=8.43 Hz), 7.42 (3H, d, J=5.76 Hz), 7.98 (2H, t, J=2.37 Hz).

Yield: 21.4%

### Example 4

### Methyl-2-methyl-5-cis-[4-(5-methyl-2-phenyl-oxazol-4-ylmethoxy)-benzyl]-[1,3]dioxane-2-carboxylate

¹H NMR: 1.5 (3H, s), 2.3 (3H, m), 2.4 (3H, s), 3.5 (1H, t, J=11.3 Hz), 3.7 (1H, m), 3.8 (3H, s), 3.9 (2H, m), 4.9 (2H, s), 6.9 (2H, t, J=7.9 Hz), 7.0 (2H, d, J=8.5 Hz), 7.4 (3H, m), 8.0 (2H, m).

Yield: 62.0%

### Example 5

### Methyl-2-methyl-5-cis-[4-(1-pyridin-2-yl-pyrrolidin-2-ylmethoxy)-benzyl]-[1,3]dioxane-2-carboxylate

¹H NMR: 1.5 (3H, s), 2.1 (2H, m), 2.2 (2H, m), 2.26 (3H, m), 3.3 (2H, m), 3.5 (3H, m), 3.8 (3H, s), 3.9 (2H, m), 4.2 (1H, dd, J=9.1 & 3.1 Hz), 4.5 (1H, m), 6.4 (1H, d, J=8.5 Hz), 6.5 (1H, t, J=5.9 Hz), 6.9 (2H, d, J=8.5 Hz), 7.0 (2H, d, J=8.5 Hz), 7.4 (1H, m), 8.1 (1H, d, J=4.0 Hz).

Yield: 44%

### Example 6

### Methyl-2-methyl-5-cis-{4-[2-(methyl-pyridin-2-yl-amino)-ethoxy]-benzyl}-[1,3]dioxane-2-carboxylate

¹H NMR: 1.5 (3H, s), 2.3 (3H, m), 3.1 (3H, s), 3.4 (2H, m), 3.7 (2H, m), 3.9 (3H, s), 3.9 (231, t, J=5.6 Hz), 4.1 (2H, t, J=5.5 Hz), 6.5 (2H, m), 6.8 (2H, d, J=3.4 Hz), 7.0 (2H, d, J=8.4 Hz), 7.4 (1H, m), 8.1 (1H, d, J=4.3 Hz).

Yield: 76%

### Example 7

### Methyl 2-methyl-5-[4-(3-methyl-4-oxo-3,4-dihydro-quinazolin-2-ylmethoxy)-benzyl]-[1,3]dioxane-2-carboxylate

¹H NMR: 1.49 (3H, s), 2.28 (2H, s), 2.94-2.97 (1H, d, J=7.86 Hz), 3.46 (2H, t, J=12.39 Hz), 3.74 (3H, s), 3.82-3.94 (5H, s) 5.15 (2H, d, J=2.16 Hz), 6.95-7.05 (1H, d, J=8.46 Hz), 7.14-7.17 (1H, d, J=8.46 Hz), 7.51 (1H, t, J=6.75 Hz), 7.69-7.79 (2H, m), 8.30 (1H, t, J=7.92 Hz).

Yield: 56.8%

### Example 8

### Methyl-2-methyl-5-cis-[4-(5-methyl-2-p-tolyl-oxazol-4-ylmethoxy)-benzyl]-[1,3]dioxane-2-carboxylate

¹H NMR: 1.5 (3H, s), 2.2 (3H, m), 2.39 (3H, s), 2.41 (3H, s), 3.4 (2H, t, J=10.9 Hz), 3.8 (3H, s), 3.9 (2H, m), 4.9 (2H, s), 6.9 (23 d, J=8.5 Hz), 7.0 (2H, d, J=8.5 Hz), 7.2 (2H, m), 7.9 (2H, d, J=8.1 Hz).

Yield: 97%

### Example 9

### Methyl-5-cis-[4-(2-carbazol-9-yl-ethoxy)-benzyl]-2-methyl-[1,3]dioxane-2-carboxylate

¹H NMR: 1.5 (3H, s), 2.2 (3H, m), 3.4 (2H, t, J=10.2 Hz), 3.8 (3H, s), 3.9 (2H, m), 4.3 (2H, t, J=6.0 Hz), 4.7 (2H, t, J=6.0 Hz), 6.7 (2H, d, J=8.4 Hz), 6.9 (2H, d, J=8.4 Hz), 7.2 (2H, m), 7.5 (4H, m), 8.1 (2H, d, J=7.7 Hz).

Yield: 77%

### Example 10

### Methyl-5-cis-[4-(2-indol-1-yl-ethoxy)-benzyl]-2-methyl-[1,3]dioxane-2-carboxylate

¹H NMR: 1.5 (3H, s), 2.26 (3H, m), 3.4 (2H, m), 3.9 (5H, m), 4.2 (2H, t, J=5.57 Hz), 4.5 (2H, t, J=5.66 Hz), 6.5 (1H, d, J=2.5 Hz), 6.7 (2H, d, J=7.1 Hz), 6.9 (2H, d, J=8.3 Hz), 7.1 (1H, t, J=7.4 Hz), 7.2 (2H, d, J=3.3 Hz), 7.4 (1H, m), 7.6 (1H, d, J=7.8 Hz).

Yield: 55.5%

### Example 11

### Methyl-5-{4-[2-(2,3-dihydro-benzo[1,4]oxazin-4-yl)-ethoxy]-benzyl}-2-methyl-[1,3]dioxane-2-carboxylate

¹H NMR: 1.5 (3H, s), 2.2 (3H, m), 2.9 (2H, m), 3.5 (2H, m), 3.7 (2H, t, J=5.7 Hz), 3.77 (2H, m), 3.8 (5H, m), 3.9 (2H, m), 4.1 (2H, m), 4.2 (2H, t, J=4.4 Hz), 6.6 (1H, m), 6.7 (1H, m), 6.7-6.8 (4H, complex), 7.0 (1H, d, J=8.5 Hz), 7.1 (1H, d, J=8.5 Hz).

Yield: 97%

### Example 12

### Methyl-2-methyl-5-cis-{4-[5-methyl-2-(5-methyl-thiophen-2-yl)-oxazol-4-ylmethoxy]-benzyl}-[1,3]dioxane-2-carboxylate

¹H NMR: 1.6 (3H, s), 2.2 (3H, m), 2.3 (3H, s), 2.5 (3H, s), 3.5 (2H, t, J=11 Hz), 3.8 (3H, s), 3.9 (2H, dd, J=12 & 3.6 Hz), 4.9 (2H, s), 6.7 (1H, m), 6.9 (2H, d, J=8.6 Hz), 7.0 (2H, d, J=8.6 Hz), 7.4 (1H, d, J=3.5 Hz).

Yield: 80%

### Example 13

### Methyl-2-methyl-5-cis-(4-{2-[5-methyl-2-(5-methyl-thiophen-2-yl)-oxazol-4-yl]-ethoxy)-benzyl}-[1,3]dioxane-2-carboxylate

¹H NMR: 1.5 (3H, s), 2.2 (3H, m), 2.3 (3H, s), 2.5 (3H, s), 2.9 (2H, t, J=6.5 Hz), 3.4 (2H, m), 3.9 (5H, m), 4.2 (2H, t, J=6.6 Hz), 6.7 (1H, m), 6.8 (2H, d, J=8.5 Hz), 7.0 (2H, d, J=8.5 Hz), 7.3 (1H, d, J=3.6 Hz).

Yield: 55.5%

### Example 14

### Methyl-2-methyl-5-cis-{4-[3-(4-phenoxy-phenoxy)-propoxy]-benzyl}-[1,3]dioxane-2-carboxylate

¹H NMR: 1.49 (3H, s), 2.21-2.35 (5H, m), 3.46 (2H, t, J=10.86 Hz), 3.82-3.91 (5H, m), 4.13 (4H, t, J=6.03 Hz), 6.81-7.06 (1H, m), 7.30 (2H, m)

Yield: 72.7%

### Example 15

### Methyl-5-cis-{4-[2-(4-Methanesulfonyloxy-phenyl)-ethoxy]-benzyl}-2-methyl-[1,3]dioxane-2-carboxylate

¹H NMR: 1.49 (3H, s), 2.27 (3H, s), 3.09 (2H, t, J=6.57 Hz), 3.13 (3H, s), 3.45 (2H, t, J=11.73 Hz), 3.82-3.92 (5H, m), 4.13 (2H, t, J=6.15 Hz), 6.79 (2H, d, J=8.46 Hz), 7.00 (2H, d, J=3.46 Hz), 7.23 (2H, d, J=8.55 Hz), 7.33 (2H, d, J=8.43 Hz).

Yield: 100%

### Example 16

### Methyl-5-cis-[4-(2-tert-Butyl-5-methyl-oxazol-4-ylmethoxy)-benzyl]-2-methyl-[1,3]dioxane-2-carboxylate

¹H NMR: 1.37 (9H, s), 1.57 (3H, s), 6.29 (6H, d, J=7.29 Hz), 3.46 (2H, m), 3.84-3.90 (5H, m), 4.85 (2H, s), 6.88 (2H, d, J=8.31 Hz), 7.00 (2H, d, J=8.01 Hz).

Yield: 100%

### Example 17

### Methyl-2-methyl-5-cis-{4-[4-methyl-2-(4-trifluoromethyl-phenyl)-thiazol-5-ylmethoxy]-benzyl}-[1,3]dioxane-2-carboxylate

¹H NMR: 1.50 (3H, s), 2.28 (3H, m), 2.50 (3H, s), 3.47 (2H, t, J=5.73 Hz), 3.84 (3H, s), 3.86-3.90 (2H, m), 5.17 (2H, s), 6.72-6.97 (2H, d, J=8.6 Hz), 7.05 (2H, d, J=8.54 Hz), 7.67 (2H, d, J=8.19 Hz), 8.01 (2H, d, J=8.28 Hz).

Yield: 95.1%

### Example 18

### Methyl-2-methyl-5-cis-{4-[2-(4-phenoxy-phenoxy)-ethoxy]-benzyl}-[1,3]dioxane-2-carboxylate

¹H NMR: 1.5 (3H, s), 2.3 (3H, m), 3.4-3.5 (2H, t, J=10.45 Hz), 3.8 (3H, s), 3.9 (2H, m), 4.3 (4H, s), 6.8-7.0 (11H, complex), 7.3 (2H, m).

Yield: 66%

### Example 19

### Methyl-5-cis-[4-(2-fluoro-benzyloxy)-benzyl]-2-methyl-[1,3]dioxane-2-carboxylate

¹H NMR: 1.5 (3H, s), 2.3 (3H, m), 3.4-3.5 (2H, t, J=10.8 Hz), 3.8 (3H, s), 3.9 (2H, m), 5.1 (2H, s), 6.9 (2H, d, J=8.5 Hz), 7.0 (2H, d, J=8.5 Hz), 7.1 (2H, m), 7.3 (1H, m), 7.5 (1H, m).

Yield: 79%

### Example 20

### Methyl-2-methyl-5-[4-(2-phenoxazin-10-yl-ethoxy)-benzyl]-[1,3]dioxane-2-carboxylate

¹H NMR: 1.5 (3H, s), 2.27 (3H, m), 3.4 (2H, t, J=10.7 Hz), 3.9-4.0 (7H, m), 4.1 (2H, t, J=6.6 Hz), 6.6 (6H, m), 6.7 (4H, m), 7.0 (2H, d, J=8.4 Hz).

Yield: 93%

### Example 21

### Methyl-2-methyl-5-cis-[4-(2-oxo-3-phenyl-oxazolidin-5-ylmethoxy)-benzyl]-[1,3]dioxane-2-carboxylate

¹H NMR: 1.5 (3H, s), 2.28 (3H, m), 3.4 (2H, s), 3.8 (5H, m), 4.0 (1H, m), 4.2 (31H, m), 4.9 (1H, m), 6.8 (2H, d, J=8.4 Hz), 7.0 (2H, d, J=8.3 Hz), 7.1 (1H, t, J=7.3 Hz), 7.4 (2H, t, J=7.5 Hz), 7.5 (2H, d, J=7.9 Hz).

Yield: 75.0%

### Example 22

### Methyl-5-cis-{4-[2-(2,3-dihydro-benzo[1,4]thiazin-4-yl)-ethoxy]-benzyl}-2-methyl-[1,3]dioxane-2-carboxylate

¹H NMR: 1.49 (3H, s), 2.25 (3H, s), 3.04 (2H, d, J=5.11 & 2.83 Hz), 3.5 (2H, t), 3.70-3.90 (9H, m), 4.14 (2H, t, J=5.85 Hz), 6.62 (1H, t), 6.71 (1H, d, J=7.98 Hz), 6.79 (2H, d, J=8.58 Hz), 6.95-7.10 (4H, m).

Yield: 88.2%

### Example 23

### Methyl-2-methyl-5-cis-[4-(2-phenothiazin-10-yl-ethoxy)-benzyl]-[1,3]dioxane-2-carboxylate

¹H NMR: 1.49 (3H, s), 2.26 (3H, s), 3.45 (2H, t, J=10.89 Hz), 3.77-3.94 (5H, m), 4.29 (4H, s), 6.80 (2H, d, J=8.58 Hz), 6.91-6.97 (6H, m), 7.09-7.31 (4H, m).

Yield: 100%

### Example 24

### Methyl-5-cis-{4-[2-(4-hexyl-3-oxo-3,4-dihydro-2H-benzo[1,4]oxazin-2-yl)-ethoxy]-benzyl}-2-methyl-[1,3]dioxane-2-carboxylate

¹H NMR: 0.88 (3H, m), 1.33 (6H, m), 1.50 (3H, s), 1.65 (2H, m), 2.19-2.27 (5H, m), 3.48 (2H, m), 3.84-3.93 (7H, m), 4.15-4.20 (2H, m), 4.77 (1H, m), 6.81 (2H, d, J=8.58 Hz), 6.91-7.32 (6H, m).

Yield: 60%

### Example 25

### Methyl-2-methyl-5-cis-(4-{2-[2-methyl-5-(4-methylsulfanyl-phenyl)-pyrrol-1-yl]-ethoxy}-benzyl)-[1,3]dioxane-2-carboxylate

¹H NMR: 1.49 (3H, s), 2.24 (3H, s), 2.36 (3H, s), 2.51 (3H, s), 3.44 (2H, t, J=10.8 Hz) 3.83 (3H, s), 3.87-3.93 (4H, m), 4.26 (2H, t, J=6.57 Hz), 5.95 (it d, J=3.12 Hz), 6.03 (1H, d, J=3.36 Hz), 6.58 (2H, d, J=8.49 Hz), 6.93 (2H, d, J=3.43 Hz), 7.26-7.33 (4H, m).

Yield: 47.6%.

### Example 26

### Methyl-5-cis-{4-[2-(2-tert-Butyl-5-methyl-oxazol-4-yl)-ethoxy]-benzyl}-2-methyl-[1,3]dioxane-2-carboxylate

¹H NMR: 1.33 (9H, s), 1.49 (3H, s), 2.20 (3H, s), 2.23 (3H, s), 2.86 (2H, t, J=6.75 Hz), 3.45 (2H, t, J=10.44 Hz), 3.84-3.90 (5H, m), 4.12 (2H, t, J=6.63 Hz), 6.76 (2H, dd, J=13.71 & 8.55 Hz), 6.94-7.00 (2H, m).

Yield: 100%.

### Example 27

### (Z)-Methyl-2-methyl-5-cis-{4-[2-(phenyl-pyridin-2-yl-methyleneaminooxy)-ethoxy]-benzyl}-[1,3]dioxane-2-carboxylate

¹H NMR: 1.49 (3H, s), 2.04 (3H, s), 3.46 (2H, t, J=10.65 Hz), 3.84 (3H, s), 3.88 (2H, dd, J=11.7 & 3.27 Hz), 4.25 (2H, t, J=5.07 Hz), 4.57 (2H, t, J=4.62 Hz), 6.83 (2H, d, J=8.58 Hz), 6.99 (2H, d, J=8.55 Hz), 7.27-7.29 (1H, m), 7.40 (5H, m), 7.66-7.73 (2H, m), 8.61 (1H, d, J=4.59 Hz).

Yield: 72.5%.

### Example 28

### (E)-Methyl-2-methyl-5-cis-{4-[2-(phenyl-pyridin-2-yl-methyleneaminooxy)-ethoxy]-benzyl}-[1,3]dioxane-2-carboxylate

¹H NMR: 1.49 (3H, s), 2.04 (3H, s), 3.46 (21, J=10.65 Hz), 3.84 (3H, s), 3.88 (2H, dd, J=11.7 & 3.27 Hz), 4.23 (21, J=5.10 Hz), 4.50 (2H, t, J=4.86 Hz), 6.83 (2H, d, J=8.58 Hz), 6.99 (2H, d, J=8.55), 7.29-7.36 (41H, m), 7.45 (2H, dd, J=7.44 & 1.56 Hz), 7.53 (1H, d, J=7.8 Hz) 7.60 (1H, m), 8.70 (1H, d, J=4.8 Hz).

Yield: 66%.

### Example 29

### Methyl-2-methyl-5-{4-[2-(5-methyl-2-p-tolyl-oxazol-4-yl)-ethoxy]-benzyl}-[1,3]dioxane-2-carboxylate

¹H NMR: 1.49 (3H, s), 2.04 (3H, s), 2.35 (3H, s), 2.38 (3H, s), 2.95 (2H, t, J=6.69 Hz) 3.45 (2H, t, J=9.0 Hz), 3.84-3.90 (5H, m), 4.20 (2H, t, J=13.5 Hz), 6.74 (2H, d, J=8.43 Hz), 6.80 (2H, d, J=8.55 Hz), 6.97 (2H, dd, J=8.37 & 6.12 Hz), 7.85 (2H, d, J=8.16 Hz).

Yield: 79%

### Example 30

### Methyl-5-cis-[4-(5-tert-Butyl-2-p-tolyl-2H-pyrazol-3-ylmethoxy)-benzyl]-2-methyl-[1,3]dioxane-2-carboxylate

¹H NMR: 1.3 (9H, s), 1.5 (3H, s), 2.3 (3H, m), 2.4 (3H, s), 3.5 (2H, t, J=11 Hz), 3.8 (3H, s), 3.9 (2H, dd, J=12 & 3 Hz), 4.8 (2H, s), 6.4 (1H, s), 6.8 (2H, d, J=8.5 Hz), 7.0 (2H, d, J=8.5 Hz), 7.2 (2H, d, J=8.2 Hz), 7.4 (2H, d, J=8.3 Hz).

Yield: 50%.

### Example 31

### Methyl-5-[6-(2-fluoro-benzyloxy)-naphthalen-2-ylmethyl]-2-methyl-[1,3]dioxane-2-carboxylate

¹H NMR: 1.50 (2H, s), 1.61 (1H, s), 2.44-2.46 (2H, m), 3.14 (1H, d, J=7.95 Hz), 3.53 (1H, t, J=10.92 Hz), 3.80-3.97 (6H, m), 5.24 (2H, s), 7.08-7.34 (6H, m), 7.47 (1H, s), 7.53-7.69 (3H, s).

Yield: 60%.

### Example 32

### Methyl-5-[6-(benzyloxy)-naphthalen-2-ylmethyl]-2-methyl-[1,3]dioxane-2-carboxylate

¹H NMR: 1.50 (2H, s), 1.61 (1H, s), 2.48 (2H, m), 3.12 (1H, d, J=7.68 Hz), 3.53 (1H, t, J=10.77 Hz), 3.80-3.97 (6H, m), 5.17 (2H, s), 7.20-7.68 (11H, m).

Yield: 60%.

### Example 33

### Methyl-2-methyl-5-cis-[4-(5-methyl-2-phenyl-oxazol-4-ylmethoxy)-phenyl]-[1,3]dioxane-2-carboxylate

### Step 1: Preparation of Methyl-5-cis-(4-benzyloxy-phenyl)-2-methyl-[1,3]dioxane-2-carboxylate

2-(4-Benzyloxy-phenyl)-propane-1,3-diol (40 g) was dissolved in 200 mL of acetonitrile, and 56.4 mL of methyl pyruvate was added. To the mixture, 39.2 mL of boron trifluoride diethyl ether complete (98%) was added with stirring at ambient temperature, and stirring was continued for 2 hours at ambient temperature. The reaction mixture was poured into an aqueous solution of sodium bicarbonate and extracted with ethyl acetate. The organic extract was washed with water, dried over sodium sulfate and evaporated under reduced pressure. The crude product was flash chromatographed over silica gel using 7% ethyl acetate in petroleum ether as eluent and the fractions eluted earlier were evaporated to obtain 19.3 g of pure product.

### Step 2: Preparation of Methyl-5-cis-(4-hydroxy-phenyl)-2-methyl-[1,3]dioxane-2-carboxylate

To a suspension of 10% palladium on charcoal (2.0 g) in methanol (100 mL) was added Methyl-5-cis-(4-benzyloxy-phenyl)-2-methyl-[1,3]dioxane-2-carboxylate (19.3 g) prepared in step 1 above followed by ammonium formate (14.2 g) and the reaction mixture was heated to reflux and continued heating. The reaction mixture was cooled to ambient temperature and the catalyst was filtered off. The filtrate was evaporated, the residue was taken in ethyl acetate and washed with water. The organic extract was dried over sodium sulfate and evaporated under reduced pressure to yield 13.7 g of product.

### Step 3: Methyl-2-methyl-5-cis-[4-(5-methyl-2-phenyl-oxazol-4-ylmethoxy)-phenyl]-[1,3]dioxane-2-carboxylate

A mixture of Methyl-5-cis-(4-hydroxy-phenyl)-2-methyl-[1,3]dioxane-2-carboxylate (prepared in step 2 above) (600 mg) 4-Chloromethyl-5-methyl-2-phenyl-oxazole (494 mg) and potassium carbonate (657 mg) in anhydrous dimethyl formamide wag stirred at 55° C. for 18 hours in an inert atmosphere. The reaction mixture was cooled to ambient temperature, poured into ice cold water and extracted with ethyl acetate. The combined organic extract was washed with water, brine solution, dried over sodium sulphate and evaporated under reduced pressure. Crude product was flash chromatographed over silica gel using ethyl acetate in petroleum ether as eluent to obtain 850 mg of pure product.

¹H NMR: 1.58 (3H, s), 2.42 (3H, s), 3.2 (1H, m), 3.8 (2H, d, J=11.8 Hz), 3.88 (3H, s), 4.05 (2H, dd, J=4.6 & 11.8 Hz), 4.96 (2H, s), 6.95 (2H, d, J=8.6 Hz), 7.0 (2H, d, J=8.6 Hz), 7.4 (3H, m), 8.00 (2H, m).

Yield: 34%

### Example 34

### Methyl-2-methyl-5-trans-[4-(5-methyl-2-phenyl-oxazol-4-ylmethoxy)-phenyl]-[1,3]dioxane-2-carboxylate

### Step 1: Preparation of Methyl-5-trans-(4-benzyloxy-phenyl)-2-methyl-[1,3]dioxane-2-carboxylate

The fractions eluted later in step 1 of example 33 were evaporated to obtain 24.0 g of pure product.

### Step 2: Preparation of Methyl-5-trans-(4-hydroxy-phenyl)-2-methyl-[1,3]dioxane-2-carboxylate

To a suspension of 10% palladium on charcoal (2.6 g) in methanol (100 mL) was added Methyl-5-trans-(4-benzyloxy-phenyl)-2-methyl-[1,3]dioxane-2-carboxylate (26 g) prepared in step 1 above followed by ammonium formate (19.16 g) and the reaction mixture was heated to reflux for 1 hour. The reaction mixture was cooled to ambient temperature and the catalyst was filtered off. The filtrate was evaporated, the residue was taken in ethyl acetate and washed with water. The organic extract was dried over sodium sulfate and evaporated under reduced pressure to yield 17.2 g of product.

### Step 3: Methyl-2-methyl-5-trans-[4-(5-methyl-2-phenyl-oxazol-4-ylmethoxy)-phenyl]-[1,3]dioxane-2-carboxylate

A mixture of Methyl-5-trans-(4-hydroxy-phenyl)-2-methyl-[1,3]dioxane-2-carboxylate (prepared in step 2 above) (1.2 g) 4-Chloromethyl-5-methyl-2-phenyl-oxazole (1.0 g) and potassium carbonate (1.3 g) in anhydrous dimethyl formamide (10 mL) was stirred at 55° C. for 18 hours in an inert atmosphere. The reaction mixture was cooled to ambient temperature, poured into ice cold water and extracted with ethyl acetate. The combined organic extract was washed with water, brine solution, dried over sodium sulphate and evaporated under reduced pressure. The crude product was recrystallised from a mixture of ethyl acetate and petroleum ether to obtain 1.3 g of pure product.

¹H NMR: 1.6 (3H, s), 2.43 (3H, s), 2.7 (1H, m), 3.86 (3H, s), 4.1 (2H, dd, J=2.9 & 12.3 Hz), 4.25 (2H, dd, J=3.7 & 12.0 Hz), 4.99 (2H, s), 6.98 (2H, d, J=8.7 Hz), 7.4 (5H, m), 8.0 (2H, m).

Yield: 68.9%.

The following compounds are prepared by procedure similar to that described in example 33 or 34 with appropriate variations of reactants, reaction conditions and quantities of reagents.

### Example 35

### Methyl-2-methyl-5-cis-{4-[2-(5-methyl-2-phenyl-oxazol-4-yl)-ethoxy]-phenyl}-[1,3]dioxane-2-carboxylate

¹H NMR: 1.57 (3H, s), 2.36 (3H, s), 2.95 (2H, d, J=6.6 Hz), 3.21 (1H, m), 3.8 (2H, d, J=11.8 Hz), 3.87 (3H, s), 4.05 (2H, dd, J=4.7 & 11.8 Hz), 4.2 (2H, t, J=6.6 Hz), 6.8 (2H, d, J=8.6 Hz), 7.0 (2H, d, J=8.6 Hz), 7.4 (3H, m), 7.98 (2H, m).

Yield: 57.7%.

### Example 36

### Methyl-2-methyl-5-cis-[4-(2-phenoxazin-10-yl-ethoxy)-phenyl]-[1,3]dioxane-2-carboxylate

¹H NMR: 1.57 (3H, s), 3.2 (1H, m), 3.8 (2H, d, J=11.76 Hz), 3.88 (3H, s), 3.95 (2H, t, J=6.5 Hz), 4.05 (2H, dd, J=4.6 & 11.8 Hz), 4.15 (2H, t, J=6.5 Hz), 6.6 (6H, m), 6.8 (4H, m), 7.0 (2H, d, J=8.5 Hz).

Yield: 38.3%.

### Example 37

### Methyl-2-methyl-5-cis-[4-(3-methyl-4-oxo-3,4-dihydro-quinazolin-2-ylmethoxy)-phenyl]-[1,3]dioxane-2-carboxylate

¹H NMR: 1.57 (3H, s), 3.2 (1H, m), 3.72 (3H, s), 3.80 (2H, d, J=11.8 Hz), 3.87 (3H, s), 4.05 (2H, dd, J=4.6 & 11.9 Hz), 5.16 (2H, s), 6.99 (2H, d, J=8.7 Hz), 7.08 (2H, d, J=8.7 Hz), 7.5 (1H, m), 7.75 (2H, m), 8.3 (1H, d, J=7.8 Hz).

Yield: 61.4%.

### Example 38

### Methyl-5-cis-[4-(2-indol-1-yl-ethoxy)-phenyl]-2-methyl-[1,3]dioxane-2-carboxylate

¹H NMR: 1.57 (3H, s), 3.15 (1H, m), 3.8 (2H, d, J=11.79 Hz), 3.87 (3H, s), 4.0 (2H, dd, J=4.6 & 11.8 Hz), 4.23 (2H, t, J=6.5 Hz), 4.5 (2H, t, J=5.6 Hz), 6.5 (1H, d, J=3.0 Hz), 6.78 (2H, d, J=9.5 Hz), 7.0 (2H, d, J=8.5 Hz), 7.1 (1H, t, J=7.4 Hz), 7.2 (2H, m), 7.39 (1H, d, J=8.19 Hz), 7.6 (1H, d, J=7.8 Hz).

Yield: 53.2%.

### Example 39

### Methyl-5-cis-{4-[2-(5-ethyl-pyridin-2-yl)-ethoxy]-phenyl}-2-methyl-[1,3]dioxane-2-carboxylate

¹H NMR: 1.23 (3H, t, J=7.6 Hz), 1.57 (3H, s), 2.63 (2H, q, J=7.6 Hz), 3.20 (3H, m), 3.79 (2H, d, 11.78 Hz), 3.87 (3H, s), 4.0 (2H, dd, J=4.6& 11.9 Hz), 4.3 (2H, t, J=6.6 Hz), 6.82 (2H, d, J=8.56 Hz), 7.06 (2H, d, J=8.56 Hz), 7.18 (1H, d, J=8.09 Hz), 7.45 (1H, dd, J=1.85 & 7.83 Hz), 8.38 (1H, s).

Yield: 27.27%

### Example 40

### Methyl-2-methyl-5-cis-[4-(5-methyl-2-p-tolyl-oxazol-4-ylmethoxy)-phenyl]-[1,3]dioxane-2-carboxylate

¹H NMR: 1.58 (3H, s), 2.40 (3H, s), 2.42 (3H, s), 3.2 (1H, m), 3.8 (2H, d, 11.6 Hz), 3.88 (3H, s), 4.05 (2H, dd, J=4.6 & 11.8 Hz), 5.00 (2H, s), 6.95 (2H, d, J=8.6 Hz), 7.0 (2H, d, J=8.6 Hz), 7.25 (2H, d, J=7.65 Hz), 7.96 (2H, d, J=8.0 Hz).

Yield: 90%.

### Example 41

### Methyl-2-Methyl-5-cis-{4-[2-(5-methyl-2-p-tolyl-oxazol-4-yl)-ethoxy]-phenyl}-[1,3]dioxane-2-carboxylate

¹H NMR: 1.58 (3H, s), 2.35 (3H, s), 2.38 (3H, s), 2.95 (2H, t, J=6.6 Hz), 3.2 (1H, m), 3.8 (2H, t, J=11.9 Hz), 3.88 (3H, s), 4.05 (2H, dd, J=4.7 & 12.0 Hz), 4.2 (2H, t, J=6.7 Hz), 6.8 (2H, d, J=8.6 Hz), 7.0 (2H, d, J=8.6 Hz), 7.24 (2H, d, J=8.0 Hz), 7.86 (2H, d, J=8.16 Hz).

Yield: 42.8%

### Example 42

### Methyl-5-cis-{4-[2-(4-methanesulfonyloxy-phenyl)-ethoxy]-phenyl}-2-methyl-[1,3]dioxane-2-carboxylate

¹H NMR: 1.58 (3H, s), 3.0 (2H, t, J=6.6 Hz), 3.13 (3H, s), 3.2 (1H, m), 3.8 (2H, d, J=11.8 Hz), 3.88 (3H, s), 4.0 (2H, dd, J=4.6 & 11.9 Hz), 4.13 (2H, t, J=6.6 Hz), 6.8 (2H, d, J=8.5 Hz), 7.0 (2H, d, J=8.5 Hz), 7.2 (2H, d, J=8.5 Hz), 7.33 (2H, d, J=8.5 Hz).

Yield: >99%.

### Example 43

### Methyl-2-methyl-5-trans-[4-(5-methyl-2-p-tolyl-oxazol-4-ylmethoxy)-phenyl]-[1,3]dioxane-2-carboxylate

¹H NMR: 1.6 (3H, s), 2.39 (3H, s), 2.42 (3H, t, s), 2.7 (1H, m), 3.86 (3H, s), 4.1 (2H, dd, J=2.9 & 12.3 Hz), 4.25 (2H, dd, J=3.7 & 12.0 Hz), 4.97 (2H, s), 7.0 (2H, d, J=3.64 Hz), 7.22 (2H, d, J=−8.1 Hz), 7.4 (2H, d, J=8.61 Hz), 7.9 (2H, d, J=8.64 Hz).

Yield: 98.3%.

### Example 44

### Methyl-2-methyl-5-trans-{4-[2-(5-methyl-2-phenyl-oxazol-4-yl)-ethoxy]-phenyl}-[1,3]dioxane-2-carboxylate

¹H NMR: 1.59 (3H, s), 2.38 (3H, s), 2.7 (1H, m), 3.0 (2H, t, J=6.6 Hz), 3.86 (3H, s), 4.0 (2H, dd, J=2.8 & 12.1 Hz), 4.2 (4H, m), 6.8 (2H, d, J=8.6 Hz), 7.4 (5H, m), 7.99 (2H, m).

Yield: 80.7%.

### Example 45

### Methyl-2-methyl-5-trans-{4-[2-(5-methyl-2-p-tolyl-oxazol-4-yl)-ethoxy]-phenyl}-[1,3]dioxane-2-carboxylate

¹H NMR: 1.6 (3H, s), 2.35 (3H, s), 2.37 (3H, s), 2.7 (1H, m), 2.9 (2H, m), 3.86 (3H, s), 4.04 (2H, dd, J=2.16 & 11.97 Hz), 4.2 (4H, m), 6.8 (2H, d, J=8.6 Hz), 7.2 (2H, d, J=8.0 Hz), 7.3 (2H, d, J=9.1 Hz), 7.87 (2H, d, J=−8.1 Hz).

Yield: 87.4%.

### Example 46

### Methyl-2-methyl-5-trans-{4-[4-methyl-2-(4-trifluoromethyl-phenyl)-thiazol-5-ylmethoxy]-phenyl}-[1,3]dioxane-2-carboxylate

¹H NMR: 1.6 (3H, s), 2.52 (3H, s), 2.7 (1H, m), 3.87 (3H, s), 4.1 (2H, dd, J=2.2 & 12.12 Hz), 4.2 (2H, dd, J=3.6 & 11.9 Hz), 5.2 (2H, s), 6.9 (2H, d, J=8.6 Hz), 7.5 (2H, d, J=8.6 Hz), 7.6 (2H, d, J=8.2 Hz), 8.0 (2H, d, J=8.1 Hz).

Yield: 61.2%.

### Example 47

### Methyl-2-methyl-5-cis-{4-[4-methyl-2-(4-trifluoromethyl-phenyl)-thiazol-5-ylmethoxy]-phenyl}-[1,3]dioxane-2-carboxylate

¹H NMR: 1.53 (3H, s), 2.5 (3H, s), 3.2 (1H, m), 3.8 (5H, m), 4.0 (2H, m), 5.1 (2H, s), 6.9 (2H, d, J=8.5 Hz), 7.0 (2H, d, J=8.5 Hz), 7.6 (2H, d, J=8.1 Hz), 8.0 (2H, d, J=8.1 Hz).

Yield: 57.5%.

### Example 48

### Methyl-2-methyl-5-cis-(4-(2-[2-methyl-5-(4-methylsulfanyl-phenyl)-pyrrol-1-yl]-ethoxy)-phenyl)-[1,3]dioxane-2-carboxylate

¹H NMR: 1.56 (3H, s), 2.35 (3H, s), 2.52 (s, 3H), 3.15 (1H, m), 3.9 (7H, m), 4.0 (2H, dd, J=4.6 & 11.9 Hz), 4.25 (2H, t, J=6.6 Hz), 5.95 (1H, d, J=2.8 Hz), 6.08 (1H, d, J=3.3 Hz), 6.5 (2H, d, J=8.6 Hz), 6.9 (2H, d, J=3.6 Hz), 7.3 (4H, m).

Yield: 74.5%

### Example 49

### Methyl-5-cis-[4-(2-tert-Butyl-5-methyl-oxazol-4-ylmethoxy)-phenyl]-2-methyl-[1,3]dioxane-2-carboxylate

¹H NMR: 1.36 (9H, s), 1.58 (3H, s), 2.30 (s, 3H), 3.2 (1H, m), 3.80 (2H, d, J=11.8 Hz), 3.95 (3H, s), 4.0 (2H, dd, J=4.5 & 11.8 Hz), 5.3 (2H, s), 6.9 (2H, d, J=8.6 Hz), 7.0 (2H, d, J=8.6 Hz).

Yield: 95.3%.

### Example 50

### Methyl-5-trans-[4-(2-tert-Butyl-5-methyl-oxazol-4-ylmethoxy)-phenyl]-2-methyl-[1,3]dioxane-2-carboxylate

¹H NMR: 1.37 (9H, s), 1.6 (3H, s), 2.31 (s, 3H), 2.7 (1H, m), 3.86 (3H, s), 4.0 (2H, dd, J=2.7 & 12.1 Hz), 4.2 (2H, dd, J=3.7 & 12.1 Hz), 4.88 (2H, s), 6.9 (2H, d, J=8.7 Hz), 7.4 (2H, d, J=8.6 Hz).

Yield: 94.5%.

### Example 51

### Methyl-2-methyl-5-cis-{4-[2-(4-phenoxy-phenoxy)-ethoxy]-phenyl}-[1,3]dioxane-2-carboxylate

¹H NMR: 1.58 (3H, s), 3.2 (1H, m), 3.8 (2H, t, J=11.8 Hz), 3.88 (3H, s), 4.0 (2H, dd, J=4.65 & 11.9 Hz), 4.29 (4H, s), 6.88-6.99 (7H, m), 7.0 (4H, d, J=8.5 Hz), 7.3 (2H, m).

Yield: 86.83%

### Example 52

### Methyl-2-methyl-5-trans-{4-[2-(4-phenoxy-phenoxy)-ethoxy]-phenyl}-[1,3]dioxane-2-carboxylate

¹H NMR: 1.60 (3H, s), 2.7 (1H, m), 3.87 (3H, s), 4.0 (2H, d, J=12.0 Hz), 4.25 (2H, dd, J=3.5 & 11.98 Hz), 4.31 (3H, s), 6.92-7.07 (9H, m), 7.3 (21H, m), 7.43 (2H, d, J=8.6 Hz).

Yield: 85.33%

### Example 53

### Methyl-5-cis-[4-(2-fluoro-benzyloxy)-phenyl]-2-methyl-[1,3]dioxane-2-carboxylate

¹H NMR: 1.58 (3H, s), 3.2 (1H, m), 3.8 (2H, d, J=11.77 Hz), 3.88 (3H, s), 4.08 (2H, dd, J=4.6 & 11.9 Hz), 5.1 (2H, s), 6.9 (2H, d, J=8.6 Hz), 7.0 (3H, d, J=8.6 Hz), 7.1 (1H, m), 7.3 (1H, m), 7.45 (1H, t, 7.3 Hz).

Yield: 78.8%.

### Example 54

### Methyl-5-trans-[4-(2-fluoro-benzyloxy)-phenyl]-2-methyl-[1,3]dioxane-2-carboxylate

¹H NMR: 1.60 (3H, s), 2.7 (1H, m), 3.86 (3H, s), 4.0 (2H, d, J=12.01 Hz), 4.25 (2H, dd, J=3.57 & 11.97 Hz), 5.1 (2H, s), 6.9 (2H, d, J=8.6 Hz), 7.05-7.18 (2H, m), 7.3 (1H, m), 7.4 (2H, d, J=8.6 Hz), 7.5 (1H, t, 7.3 Hz).

Yield: 78.5%.

### Example 55

### Methyl-5-cis-[4-(5-tert-Butyl-2-p-tolyl-2H-pyrazol-3-ylmethoxy)-phenyl]-2-methyl-[1,3]dioxane-2-carboxylate

¹H NMR: 1.35 (9H, t, s), 1.58 (3H, s), 2.38 (3H, s), 3.2 (1H, m), 3.85 (2H, d, J=11.64 Hz), 3.88 (3H, s), 4.0 (2H, dd, J=4.56 & 11.61 Hz), 4.8 (2H, s), 6.39 (1H, s), 6.88 (2H, d, J=8.43 Hz), 7.06 (2H, d, J=8.43 Hz), 7.2 (2H, d, J=8.1 Hz), 7.4 (2H, d, J=8.1 Hz).

Yield: 61.3%.

### Example 56

### Methyl-5-trans-[4-(5-tert-Butyl-2-p-tolyl-2H-pyrazol-3-ylmethoxy)-phenyl]-2-methyl-[1,3]dioxane-2-carboxylate

¹H NMR: 1.36 (9H, s), 1.60 (3H, s), 2.36 (3H, s), 2.7 (1H, m), 3.87 (3H, s), 4.06 (2H, d, J=11.1 Hz), 4.25 (2H, d, J=9.18 Hz), 4.9 (2H, s), 6.41 (1H, s), 6.90 (2H, d, J=8.4 Hz), 7.22 (2H, d, J=7.86 Hz), 7.4 (4H, t, J=7.4 Hz).

Yield: 59.9%.

### Example 57

### Methyl-2-methyl-5-trans-[4-(2-oxo-3-phenyl-oxazolidin-5-ylmethoxy)-phenyl]-[1,3]dioxane-2-carboxylate

¹H NMR: 1.59 (3H, s), 2.69 (1H, m), 3.86 (3H, s), 4.0 (3H, m), 4.2 (5H, m), 4.99 (1H, m), 6.9 (2H d, J=8.2 Hz), 7.15 (1H, m), 7.4 (4H, m), 7.59 (2H, d, J=7.78 Hz).

Yield: 66.1%.

### Example 58

### Methyl-2-methyl-5-cis-[4-(2-oxo-3-phenyl-oxazolidin-5-ylmethoxy)-phenyl]-[1,3]dioxane-2-carboxylate

¹H NMR: 1.58 (3H, s), 3.2 (1H, m), 3.84 (2H, d, J=11.82 Hz), 3.88 (3H, s), 4.0 (3H, m), 4.2 (3H, m), 4.9 (1H, m), 6.8 (2H, d, J=8.6 Hz), 7.0 (2H, d, J=8.6 Hz), 7.1 (1H, t, J=7.4 Hz), 7.3 (2H, m), 7.5 (2H, d, J=7.98 Hz).

Yield: 73.9%

### Example 59

### Methyl-5-trans-{4-[2-(4-Methanesulfonyloxy-phenyl)-ethoxy]-phenyl}-2-methyl-[1,3]dioxane-2-carboxylate

¹H NMR: 1.59 (3H, s), 2.69 (1H, m), 3.1 (5H, m), 3.86 (3H, s), 4.05 (2H, d, J=11.0 Hz), 4.14-4.25 (4H, m), 6.8 (2H, d, J=8.4 Hz), 7.2 (2H, d, J=8.4 Hz), 7.33-7.41 (4H, m).

Yield: 68.9%.

### Example 60

### Methyl-{2-methyl-5-trans-[4-(3-methyl-4-oxo-3,4-dihydro-quinazolin-2-ylmethoxy)-phenyl]-[1,3]dioxane-2 carboxylate

¹H NMR: 1.59 (3H, s), 2.7 (1H, m), 3.72 (3H, s), 3.86 (3H, s), 4.0 (2H, dd, J=2.2 & 12.17 Hz), 4.2 (2H, dd, J=3.6 & 12.69 Hz), 5.18 (2H, s), 7.0 (2H, d, J=8.7 Hz), 7.4 (2H, d, J=8.7 Hz), 7.5 (1H, m), 7.7 (2H, m), 8.3 (1H, d, J=7.83 Hz).

Yield: 59.43%

### Example 61

### 2-Methyl-5-{4-[2-(5-methyl-2-phenyl-oxazol-4-yl)-ethoxy]-benzyl}-[1,3]dioxane-2-carboxylic acid

To a solution of Methyl-2-methyl-5-{4-[2-(5-methyl-2-phenyl-oxazol-4-yl)-ethoxy]-benzyl}-[1,3]dioxane-2-carboxylate (263 mg) in methanol was added a solution of sodium hydroxide (46.5 mg) in water and the reaction mixture was stirred at ambient temperature for 15 hours. The solvents were evaporated and the residue was dissolved in water, acidified with 1N HCl and extracted with ethyl acetate. The combined organic extract was washed with water, brine, dried over sodium sulphate and evaporated under reduced pressure. The crude product was flash chromatographed over silica gel using 2% methanol in chloroform as eluent to obtain 160 mg of pure product.

¹H NMR: 1.54 (3H, s), 2.25 (3H, s), 2.38 (3H, s), 2.86 (1H, m), 2.96-3.03 (2H, m), 3.52-3.57 (2H, m), 3.90-3.93 (2H, m), 4.21 (2H, t, J=6.9 Hz), 6.82 (2H, d, J=3.43 Hz), 7.07 (2H, d, J=8.49 Hz), 7.32-7.43 (3H, m), 7.96-7.99 (2H, m).

Yield: 63.0%

The following compounds were prepared by a procedure similar to those described in example 57 with appropriate variations of reactants, reaction conditions and quantities of reagents.

### Example 62

### 2-Methyl-5-{4-[2-(5-methyl-2-p-tolyl-oxazol-4-yl)-ethoxy]-benzyl}-[1,3]dioxane-2-carboxylic acid

¹H NMR: 1.55 (3H, s), 2.24 (2H, s), 2.37 (3H, s), 2.38 (3H, s), 2.86 (1H, d, J=7.77 Hz), 2.96-3.03 (2H, m), 3.49-3.72 (2H, m), 3.87-3.92 (2H, m), 4.20 (2H, t, J=6.72 Hz), 6.78-6.85 (2H, m), 6.93-7.09 (2H, m), 7.22 (2H, m), 7.85-7.87 (2H, m).

Yield: 58.4%

### Example 63

### 2-Methyl-5-cis-[4-(5-methyl-2-phenyl-oxazol-4-ylmethoxy)-benzyl]-[1,3]dioxane-2-carboxylic acid

¹H NMR: 1.52 (3H, s), 2.29 (3H, m), 2.43 (3H, s), 3.6 (2H, t, J=10.8 Hz), 3.9 (2H, m), 4.96 (2H, s), 6.9 (2H, d, J=8.5 Hz), 7.0 (2H, d, J=8.5 Hz), 7.4 (3H, m), 8.0 (2H, m).

Yield: 85.0%

### Example 64

### 2-Methyl-5-cis-[4-(1-pyridin-2-yl-pyrrolidin-2-ylmethoxy)-benzyl]-[1,3]dioxane-2-carboxylic acid

¹H NMR: 1.5 (3H, s), 2.1 (3H, m), 2.2 (4H, m), 3.6 (4H, m), 3.8 (21H, m), 3.9 (1H, t), 4.1 (1H, m), 4.5 (1H, m), 6.5 (1H, d, J=8.5 Hz), 6.6 (1H, t, J=5.9 Hz), 6.8 (2H, d, J=8.31 Hz), 6.9 (2H, d, J=8.22 Hz), 7.5 (1H, t, J=7.2 Hz), 8.2 (1H, m).

Yield: 65.0%

### Example 65

### 2-Methyl-5-cis-{4-[2-(methyl-pyridin-2-yl-amino)-ethoxy]-benzyl}-[1,3]dioxane-2-carboxylic acid

¹H NMR: 1.5 (3H, s), 2.2 (3H, m), 3.1 (3H, s), 3.6 (2H, t, J=10.52 Hz), 3.8 (2H, m), 4.0 (2H, t, J=5.0 Hz), 4.2 (2H, m), 6.6 (2H, m), 6.7 (2H, d, J=8.28 Hz), 6.9 (2H, d, J=8.4 Hz), 7.5 (1H, m), 8.2 (1H, m).

Yield: 55.5%

### Example 66

### 2-Methyl-5-[4-(3-methyl-4-oxo-3,4-dihydro-quinazolin-2-ylmethoxy)-benzyl]-[1,3]dioxane-2-carboxylic acid

¹H NMR: 1.3 (3H, s), 2.1 (1H, m), 2.2 (2H, d, J=6.9 Hz), 3.4 (2H, t, J=1.3 Hz), 3.6 (3H, s), 3.7 (2H, dd, J=11.7 & 4.2 Hz), 5.2 (2H, s), 7.0 (2H, d, J=8.5 Hz), 7.1 (2H, d, J=8.3 Hz), 7.5 (1H, t, J=7.5 Hz), 7.6 (1H, d, J=8.04 Hz), 7.8 (1H, t, J=7.1 Hz), 8.1 (1H, d, J=7.8 Hz).

Yield: 95%

### Example 67

### 2-Methyl-5-[4-(2-phenoxazin-10-yl-ethoxy)-benzyl]-[1,3]dioxane-2-carboxylic acid

¹H NMR: 1.5 (3H, s), 2.26 (3H, m), 3.5 (2H, t, J=10.4 Hz), 3.9-4.0 (41H, m), 4.1 (2H, t, J=6.5 Hz), 6.6 (6H, m), 6.7 (4H, m), 7.0 (2H, d, J=8.5 Hz), 7.1 (2H, d, J=8.5 Hz).

Yield: 57.0%

### Example 68

### 2-Methyl-5-cis-[4-(5-methyl-2-p-tolyl-oxazol-4-ylmethoxy)-benzyl]-[1,3]dioxane-2-carboxylic acid

¹H NMR: 1.3 (3H, s), 2.1 (1H, m), 2.2 (2H, d, J=6.9 Hz), 2.3 (3H, s), 2.4 (3H, s), 3.4 (2H, t, J=11.4 Hz), 3.7 (2H, dd, J=11.55 & 4.1 Hz), 4.9 (2H, s), 6.9 (2H, d, J=8.4 Hz), 7.1 (2H, d, J=8.4 Hz), 7.3 (2H, d, J=8.1 Hz), 7.8 (2H, d, J=8.1 Hz).

Yield: 95.0%

### Example 69

### 5-cis-[4-(2-Carbazol-9-yl-ethoxy)-benzyl]-2-methyl-[1,3]dioxane-2-carboxylic acid

¹H NMR: 1.5 (3H, s), 2.2 (3H, m), 3.5 (2H, t, J=10.7 Hz), 3.9 (2H, m), 4.3 (2H, t, J=5.9 Hz), 4.7 (2H, t, J=5.9 Hz), 6.7 (2H, d, J=8.5 Hz), 6.9 (2H, d, J=8.5 Hz), 7.2 (2H, m), 7.5 (4H, m), 8.1 (2H, d, J=7.8 Hz).

Yield: 35.0%

### Example 70

### 5-cis-[4-(2-Indol-1-yl-ethoxy)-benzyl]-2-methyl-[1,3]dioxane-1-carboxylic acid

¹H NMR: 1.5 (3H, s), 2.2 (31H, m), 3.5 (2H, t, J=10.7 Hz), 3.9 (2H, dd, J=17.9 & 4.6 Hz), 4.2 (2H, t, J=−5.6 Hz), 4.5 (2H, t, J=5.6 Hz), 6.5 (1H, d, J=3.3 Hz), 6.7 (2H, d, J=8.6 Hz), 6.9 (2H, d, J=8.5 Hz), 7.1 (1H, m), 7.2 (2H, m), 7.4 (1H, d, J=8.1 Hz), 7.6 (1H, d, J=7.8 Hz).

Yield: 39.0%

### Example 71

### 5-{4-[2-(2,3-Dihydro-benzo[1,4]oxazin-4-yl)-ethoxy]-benzyl}-2-methyl-[1,3]dioxane-2-carboxylic acid

¹H NMR: 1.2 (3H, s) (trans), 1.5 (3H, s), 2.3 (3H, m), 2.9 (2H, m) (trans), 3.4 (2H, m) (trans), 3.5 (2H, t, J=4.2 Hz), 3.7 (2H, t, J=5.6 Hz), 3.8 (2H, m), 3.9 (2H, m), 4.1 (2H, t, J=5.6 Hz), 4.2 (2H, t, J=4.3 Hz), 6.6 (1H, t, J=7.5 Hz), 6.7 (1H, d, J=7.3 Hz), 6.7-6.8 (4H, complex), 7.1 (1H, d, J=8.4 Hz).

Yield: 66%.

### Example 72

### 2-Methyl-5-cis-{4-[5-methyl-2-(5-methyl-thiophen-2-yl)-oxazol-4-ylmethoxy]-benzyl}-[1,3]dioxane-2-carboxylic acid

¹H NMR: 1.5 (3H, s), 2.2 (3H, m), 2.4 (3H, s), 2.5 (3H, s), 3.5 (2H, t, J=11.1 Hz), 3.9 (2H, m), 4.9 (2H, s), 6.7 (1H, d, J=2.8 Hz), 6.9 (2H, d, J=8.6 Hz), 7.0 (2H, d, J=8.55 Hz), 7.4 (1H, d, J=3.6 Hz).

Yield: 64.0%

### Example 73

### 5-cis-{4-[2-(2,3-Dihydro-benzo[1,4]thiazin-4-yl)-ethoxy]-benzyl}-2-methyl-[1,3]dioxane-2-carboxylic acid

¹H NMR: 1.56 (3H, s), 2.26-2.31 (3H, m), 3.02-3.05 (2H, m), 3.54 (2H, t, J=6 Hz), 3.70-3.79 (4H, m), 3.93 (2H, dd, J=13.38 & 4.14), 4.14 (2H, t, J=5.67 Hz), 6.64 (1H, d, J=0.96 Hz), 6.71 (1H, d, J=7.74 Hz), 6.80 (2H, d, J=8.589 Hz), 6.95-7.05 (4H, m).

Yield: 76.0%

### Example 74

### 2-Methyl-5-cis-[4-(2-phenothiazin-10-yl-ethoxy)-benzyl]-[1,3]dioxane-2-carboxylic acid

¹H NMR: 1.55 (3H, s), 2.30 (3H, s), 3.50 (2H, m), 3.91 (2H, dd, J=9.69 & 4.17 Hz), 4.29 (4H, s), 6.81 (2H, d, J=3.58 Hz), 6.91-7.01 (6H, m), 7.13-7.13 (4H, m).

Yield: 64.0%

### Example 75

### 5-cis-{4-[2-(4-Hexyl-3-oxo-3,4-dihydro-2H-benzo[1,4]oxazin-2-yl)-ethoxy]-benzyl}-2-methyl-[1,3]dioxane-2-carboxylic acid

¹H NMR: 0.88 (3H, t, J=6.81 Hz), 1.25-1.37 (6H, m), 1.56 (3H, s), 1.64 (2H, m), 2.20-2.27 (5H, m), 2.32 (1H, m), 3.53 (2H, t, J=10.77 Hz), 3.88-3.95 (4H, m), 4.14-4.19 (2H, m), 4.78 (1H, dd, J=9.24 & 3.99 Hz), 6.81 (2H, d, J=8.58 Hz), 6.95-7.05 (6H, m).

Yield: 60.2%

### Example 76

### 2-Methyl-5-cis-(4-{2-[5-methyl-2-(5-methyl-thiophen-2-yl)-oxazol-4-yl]-ethoxy}-benzyl)-[1,3]dioxane-2-carboxylic acid

¹H NMR: 1.55 (3H, s), 2.2 (3H, m), 2.3 (3H, s), 2.5 (3H, s), 2.9 (2H, m), 3.5 (2H, m), 3.9 (2H, d, J=9.96 Hz), 4.2 (2H, t, J=6.3 Hz), 6.7 (1H, s), 6.8 (2H, d, J=8.2 Hz), 6.9 (2H, d, J=8.0 Hz), 7.4 (1H, s).

Yield: 46%

### Example 77

### 2-Methyl-5-{4-[2-(5-methyl-2-phenyl-oxazol yl)-ethoxy]-benzyl}-[1,3]dioxane-2-carboxylic acid

¹H NMR: 1.54 (3H, s), 2.29 (3H, s), 2.37 (3H, s), 2.97 (2H, t, J=6.69 Hz), 3.51-3.55 (2H, m), 3.91 (2H, dd, J=12.57 & 4.2 Hz), 4.21 (2H, t, J=6.72 Hz), 6.82 (2H, d, J=8.55 Hz), 6.99 (2H, d, J=8.52 Hz), 7.41 (3H, m), 7.95-7.99 (24 dd, J=7.83 & 2.88 Hz).

Yield: 32%

### Example 78

### 2-Methyl-5-cis-{4-[3-(4-phenoxy-phenoxy)-propoxy]-benzyl}-[1,3]dioxane-2-carboxylic acid

¹H NMR: 1.55 (3H, s), 2.25-2.31 (5H, m), 3.52 (2H, t, J=10.29 Hz), 3.91 (2H, d, J=9.57 Hz), 4.14 (4H, t, J=5.71 Hz), 6.82-7.05 (1H, m), 7.28 (2H, s).

Yield: 77%

### Example 79

### 5-cis-{4-[2-(4-Methanesulfonyloxy-phenyl)-ethoxy]-benzyl}-2-methyl-[1,3]dioxane-2-carboxylic acid

¹H NMR: 1.55 (3H, s), 2.27 (3H, s), 3.07-3.11 (2H, t, J=6.72 Hz), 3.13 (3H, s), 3.44-3.55 (2H, m), 3.90 (2H, dd, J=13.74 & 4.2 Hz), 4.16 (2H, t, J=6.69 Hz), 6.80 (2H, d, J=8.49 Hz), 6.99 (2H, d, J=8.49 Hz), 7.23 (2H, d, J=8.61 Hz), 7.33 (2H, d, J=8.49 Hz).

Yield: 73.1%

### Example 80

### 5-cis-[4-(2-tert-Butyl-5-methyl-oxazol 4-ylmethoxy)-benzyl]-2-methyl-[1,3]dioxane-2-carboxylic acid

¹H NMR: 1.38 (9H, s), 1.51 (3H, s), 2.12 (2H, d, J=7.2 Hz), 2.26 (1H, m), 2.33 (3H, s), 3.43 (2H, t, J=11.28 Hz), 3.82 (2H, dd, J=11.82 & 4.11 Hz), 4.90 (2H, s), 6.92 (4H, dd, J=19.11 & 3.67 Hz).

Yield: 49%

### Example 81

### 2-Methyl-5-cis-{4-[4-methyl-2-(4-trifluoromethyl-phenyl)-thiazol-5-ylmethoxy]-benzyl}-[1,3]dioxane-2-carboxylic acid

¹H NMR: 1.55 (3H, s), 2.33 (3H, s), 2.50 (3H, s), 3.53 (2H, d, J=11.01 Hz), 3.93 (2H, dd, J=14.28 & 3.87 Hz), 5.18 (2H, s), 6.90 (2H, d, J=8.55 Hz), 7.01 (2H, d, J=8.55 Hz), 7.68 (2H, d, J=8.18 Hz), 8.01 (2H, d, J=8.07 Hz).

Yield: 52.1%

### Example 82

### 2-Methyl-5-cis-{4-[2-(4-phenoxy-phenoxy)-ethoxy]-benzyl}-[1,3]dioxane-2-carboxylic acid

¹H NMR: 1.56 (3H, s), 2.3 (3H, s), 3.54 (2H, t, J=10.59 Hz), 3.93 (2H, d, J=9.33 & 3.54 Hz), 4.00 (4H, s), 6.86-7.07 (1H, m), 7.27-7.32 (2H, m).

Yield: 94%

### Example 83

### 5-cis-[4-(2-Fluoro-benzyloxy)-benzyl]-2-methyl-[1,3]dioxane-2-carboxylic acid

¹H NMR: 1.56 (3H, s), 2.32 (3H, s), 3.54 (2H, t, J=10.5 Hz), 3.91-3.94 (2H, m), 5.11 (2H, s), 6.90 (2H, d, J=8.5 Hz), 7.01-7.18 (4H, m), 7.30-7.32 (1H, m), 7.49 (1H, t, J=6.78 Hz).

Yield: 99%

### Example 84

### 2-Methyl-5-cis-(4-{2-[2-methyl-5-(4-methylsulfanyl-phenyl)-pyrrol-1-yl]-ethoxy}-benzyl)-[1,3]dioxane-2-carboxylic acid

¹H NMR: 1.54 (3H, s), 2.27 (3H, s), 2.36 (3H, s), 2.52 (3H, s), 3.51 (2H, t J=10.62 Hz), 3.91 (4H, t, J=6.48 Hz), 4.26 (2H, t, J=8.82 Hz), 5.96 (1H, d, J=2.55 Hz), 6.09 (1H, d, J=3.3 Hz), 6.60 (2H, d, J=9.0 Hz), 6.92 (2H, d, J=8.43 Hz), 7.21-7.33 (4H, m).

Yield: 79.9%

### Example 85

### 5-cis-{4-[2-(2-tert-Butyl-5-methyl-oxazol-4-yl)-ethoxy]-benzyl}-2-methyl-[1,3]dioxane-2-carboxylic acid

¹H NMR: 1.35 (9H, s), 1.54 (3H, s), 2.24 (3H, s), 2.27 (3H, s), 2.89-2.96 (2H, dd, J=15.03 & 8.13 Hz), 3.50 (2H, t, J=9.48 Hz), 3.84-3.89 (2H, m), 4.12 (2H, t, J=6.66 Hz), 6.78 (2H, d, J=8.34 Hz), 6.92 (2H, d, J=8.1 Hz).

Yield: 20%

### Example 86

### (Z)-2-Methyl-5-cis-{4-[2-(phenyl-pyridin-2-yl-methyleneaminooxy)-ethoxy]-benzyl}-[1,3]dioxane-2-carboxylic acid

¹H NMR: 1.50 (3H, s), 2.36 (3H, d, J=6.12 Hz), 3.50 (2H, t, J=11.49 Hz), 3.88 (2H, dd, J=8.79 Hz), 4.29 (2H, t), 4.55 (2H, t), 6.85 (2H, d, J=8.49 Hz), 6.97 (2H, d, J=8.25 Hz), 7.29 (3H, m), 7.39 (3H, m), 7.67-7.74 (2H, m), 8.63 (1H, d, J=4.2 Hz).

Yield: 72.4%

### Example 87

### (E)-2-Methyl-5-cis-{4-[2-(phenyl-pyridin-2-yl-methyleneaminooxy)-ethoxy]-benzyl}-[1,3]dioxane-2-carboxylic acid

¹H NMR: 1.50 (3H, s), 2.38 (3H, s), 3.49 (2H, t, J=7.62 Hz), 3.87 (2H, dd, J=9.06 Hz), 4.24 (2H, t), 4.50 (2H, t), 6.82 (2H, d, J=8.37 Hz), 6.97 (2H, d, J=8.31 Hz), 7.34 (4H, m), 7.43 (2H, d, J=7.56 Hz), 7.53 (1H, d, J=7.74 Hz), 7.80 (1H, t, J=6.54 Hz), 8.74 (1H, d, J=6.54 Hz).

Yield: 56%

### Example 88

### 2-Methyl-5-cis-[4-(5-methyl-2-phenyl-oxazol-4-ylmethoxy)-phenyl]-[1,3]dioxane-2-carboxylic acid

¹H NMR: 1.61 (3H, s), 2.46 (3H, s), 3.07 (1H, m), 3.64 (2H, t, J=11.5 Hz), 3.75 (2H, dd, J=4.8 & 11.7 Hz), 5.05 (2H, s), 69 (4H, m), 7.47 (31 ml), 3.05 (2H, m).

Yield: 51.72%

### Example 89

### 2-Methyl-5-cis{4-[2-(5-methyl-2-phenyl-oxazol-4-yl)-ethoxy]-phenyl}-[1,3]dioxane-2-carboxylic acid

¹H NMR: 1.63 (3H, s), 2.40 (3H, s), 3.06 (2H, t, J=6.5 Hz), 3.17 (1H, m), 3.89 (2H, t, J=11.5 Hz), 4.01 (2H, dd, J=4.8 & 11.6 Hz), 4.2 (2H, t, J=6.6 Hz), 6.76 (2H, d, J=8.5 Hz), 6.95 (21, J=8.5 Hz), 7.42 (3H, m), 8.00 (2H, m).

Yield: 80.93%

### Example 90

### 2-Methyl-5-cis-[4-(2-phenoxazin-10-yl-ethoxy)-phenyl]-[1,3]dioxane-2-carboxylic acid

¹H NMR: 1.65 (3H, s), 3.22 (1H, m), 3.86-3.97 (4H, m), 4.05-4.17 (4H, m), 6.6 (6H, m), 6.7-6.85 (4H, m), 7.0 (2H, d, J=8.13 Hz).

Yield: 89.2%

### Example 91

### 2-Methyl-5-cis-[4-(3-methyl-4-oxo-3,4-dihydro-quinazolin-2-ylmethoxy)-phenyl]-[1,3]dioxane-2-carboxylic acid

¹H NMR: 1.41 (3H, s), 3.1 (1H, m), 3.59 (3H, s), 3.75 (2H, t, J=11.5 Hz), 3.9 (2H, dd, J=4.5 & 11.5 Hz), 5.25 (2H, s), 7.05 (2H, d, J=8.6 Hz), 7.1 (2H, d, J=8.6 Hz), 7.56 (1H, t, J=7.4 Hz), 7.65 (1H, d, J=8.0 Hz), 7.83 (1H, t, J=7.0 Hz), 8.16 (1H, d, J=9.0 Hz).

Yield: 70.8%

### Example 92

### 5-cis-[4-(2-Indol-1-yl-ethoxy)-phenyl]-2-methyl-[1,3]dioxane-2-carboxylic acid

¹H NMR: 1.63 (3H, s), 3.21 (1H, m), 3.87 (2H, t, J=11.6 Hz), 4.0 (2H, dd, J=4.6 & 11.9 Hz), 4.25 (2H, J=5.6 Hz), 4.5 (2H, t, J=5.6 Hz), 6.5 (1H, d, J=7.76 Hz), 6.76 (2H, d, J=8.6 Hz), 7.0 (2H, d, J=8.6 Hz), 7.1 (1H, t, J=7.2 Hz), 7.2 (2H, m), 7.4 (1H, d, J=8.1 Hz), 7.64 (1H, d, J=7.86 Hz).

Yield: 69.13%

### Example 93

### 5-cis-{4-[2-(5-Ethyl-pyridin-2-yl)-methoxy]-phenyl}-2-methyl-[1,3]dioxane-2-carboxylic acid

¹H NMR: 1.3 (3H, t, J=7.1 Hz), 1.63 (3H, s), 2.7 (2H, m), 3.1 (1H, m), 3.5 (2H, m), 3.96 (4H, m), 4.3 (2H, m), 6.7 (2H, d, J=8.1 Hz), 6.95 (2H, d, J=8.1 Hz), 7.5 (1H, d, J=7.8 Hz), 7.8 (1H, d, J=6.9 Hz), 8.6 (1H, s).

Yield: 68.66%

### Example 94

### 2-Methyl-5-cis-[4-(5-methyl-2-p-tolyl-oxazol-4-ylmethoxy)-phenyl]-[1,3]dioxane-2-carboxylic acid

¹H NMR: 1.6 (3H, s), 2.4 (3H, s), 2.45 (3H, s), 3.0 (1H, m), 3.5-3.7 (4H, m), 5.05 (2H, s), 6.86 (2H, d, J=8.6 Hz), 6.96 (2H, d, J=8.6 Hz), 7.29 (2H d, J=8.0 Hz), 7.95 (2H, d, J=8.0 Hz).

Yield: 90.2%

### Example 95

### 2-Methyl-5-cis-{4-[2-(5-methyl-2-p-tolyl-oxazol-4-yl)-ethoxy]-phenyl}[1,3]dioxane-2-carboxylic acid

¹H NMR: 1.64 (3H, s), 2.38 (3H, s), 2.39 (3H, s), 3.07 (21, J=6.5 Hz), 3.16 (1H, m), 3.88 (2H, t, J=11.5 Hz), 4.00 (2H, dd, J=4.8 & 11.7 Hz), 4.19 (2H, d, J=6.6 Hz), 6.75 (2H, d, J=8.5 Hz), 6.93 (2H, d, J=8.5 Hz), 7.22 (2H, d, J=9.96 Hz), 7.86 (2H, d, J=8.1 Hz).

Yield: 94.23%

### Example 96

### 5-cis-{4-[2-(4-Methanesulfonyloxy-phenyl)-ethoxy]-phenyl}-2-methyl-[1,3]dioxane-2-carboxylic acid

¹H NMR: 1.65 (3H, s), 3.1 (2H, t, J=6.7 Hz), 3.13 (3H, s), 3.2 (1H, m), 3.9 (2H, t, J=11.7 Hz), 4.0-4.15 (4H, m), 6.8 (2H, d, J=8.5 Hz), 7.0 (2H, d, J=8.5 Hz), 7.2 (2H, d, J=8.5 Hz), 7.3 (2H, d, J=8.5 z).

Yield: 82.6%

### Example 97

### 2-Methyl-5-trans-[4-(5-methyl-2-phenyl-oxazol-4-ylmethoxy)-phenyl]-[1,3]dioxane-2-carboxylic acid

¹H NMR: 1.62 (3H, s), 2.44 (3H, s), 2.67 (1H, m), 4.05 (2H, dd, J=2.5 & 11.67 Hz), 4.2 (2H, dd, J=3.2 & 11.8 Hz), 5.02 (2H, s), 7.01 (2H, d, J=8.5 Hz), 7.5 (2H, d, J=8.5 Hz), 7.45 (3H, m), 8.02 (2H, m).

Yield: 75.85%

### Example 98

### 2-Methyl-5-trans-[4-(5-methyl-2-p-tolyl-oxazol-4-ylmethoxy)-phenyl]-[1,3]dioxane-2-carboxylic acid

¹H NMR: 1.63 (3H, s), 2.39 (3H, s), 2.43 (3H, s), 2.68 (1H, m), 4.05 (2H, dd, J=3.3 &, 11.9 Hz), 4.2 (2H, dd, J=3.7 & 11.9 Hz), 5.01 (2H, s), 7.00 (2H, d, J=8.6 Hz), 7.2 (2H, d, J=6.8 Hz), 7.39 (2H, d, J=8.6 Hz), 7.9 (2H, d, J=8.1 Hz).

Yield: 86.8%

### Example 99

### 2-Methyl-5-trans-{4-[2-(5-methyl-2-phenyl-oxazol-4-yl)-ethoxy]-phenyl}-[1,3]dioxane-2-carboxylic acid

¹H NMR: 1.65 (3H, s), 2.39 (3H, s), 2.7 (1H, m), 3.0 (2H, t, J=6.6 Hz), 4.0 (2H, dd, J=3.7 & 11.8 Hz), 4.2 (4H, t, J=6.7 Hz), 6.9 (2H, d, J=8.5 Hz), 7.3 (2H, d, J=8.5 Hz), 7.4 (3H, m), 8.0 (2H, m).

Yield: 94.9%

### Example 100

### 2-Methyl-5-trans-{4-[2-(5-methyl-2-p-tolyl-oxazol-4-yl)-ethoxy]-phenyl}-[1,3]dioxane-2-carboxylic acid

¹H NMR (CD₃OD): 1.5 (3H, s), 2.35 (3H, s), 2.37 (3H, s), 2.67 (1H, m), 2.9 (2H, t, J=6.5 Hz), 3.9 (2H, d, J=9.9 Hz), 4.2 (4H, m), 6.85 (2H, d, J=8.5 Hz), 7.26 (2H, d, J=8.1 Hz), 7.4 (2H, d, J=8.5 Hz), 7.8 (2, d, J=8.1 Hz).

Yield: 45.25%

### Example 101

### 2-Methyl-5-trans-{4-[4-methyl-2-(4-trifluoromethyl-phenyl)-thiazol-5-ylmethoxy]-phenyl}-[1,3]dioxane-2-carboxylic acid

¹H NMR: 1.67 (3H, s), 2.52 (3H, s), 2.81 (1H, m), 4.1 (2H, dd, J=3.2 & 11.7 Hz), 4.3 (2H, dd, J=3.5 & 11.9 Hz), 5.2 (2H, s), 6.95 (2H, J=8.5 Hz), 7.4 (2H, d, J=8.5 Hz), 7.66 (2H, d, J=8.1 Hz), 8.01 (2H, d, J=8.1 Hz).

Yield: 44.41%

### Example 102

### 2-Methyl-5-cis-{4-[4-methyl-2-(4-fluoromethyl-phenyl)-thiazol-5-ylmethoxy]-phenyl}-[1,3]dioxane-2-carboxylic acid

¹H NMR: 1.65 (3H, s), 2.51 (3H, s), 3.2 (1H, m), 3.9 (2H, t, J=11.6 Hz), 4.1 (2H, dd, J=4.5 & 11.8 Hz), 5.17 (2H, s), 6.9 (2H, d, J=8.55 Hz), 7.1 (2H, d, J=8.55 Hz), 7.68 (2H, d, J=8.1 Hz), 8.0 (2H, d, J=8.0 Hz).

Yield: δ 09.93%

### Example 103

### 2-Methyl-5-cis-(4-{2-[2-methyl-5-(4-methylsulfanyl-phenyl)-pyrrol-1-yl]-ethoxy}-phenyl)-[1,3]dioxane-2-carboxylic acid

¹H NMR 1.65 (3H, s), 2.35 (3H, s), 2.51 (s, 3H), 3.2 (1H, m), 3.9 (4H, m), 4.05 (2H, dd, J=4.6 & 11.9 Hz), 4.25 (2H, t, J=6.6 Hz), 5.95 (1H, d, J=3.3 Hz), 6.07 (1H, d, J=3.3 Hz), 6.55 (2H, d, J=8.6 Hz), 6.99 (2H, d, J=8.6 Hz), 7.3 (4H, m).

Yield: 88.7%

### Example 104

### 5-cis-[4-(2-tert-Butyl-5-methyl-oxazol-4-ylmethoxy)-phenyl]-2-methyl-[1,3]dioxane-2-carboxylic acid

¹H NMR: 1.41 (9H, s), 1.62 (3H, s), 2.34 (s, 3H), 3.0 (1H, m), 3.5 (2H, t, J=11.5 Hz), 3.66 (2H, dd, J=4.8 & 11.6 Hz), 4.95 (2H, s), 6.8 (2H, d, J=8.6 Hz), 6.9 (2H, d, J=8.6 Hz).

Yield: 85.5%.

### Example 105

### 5-trans-[4-(2-tert-Butyl-5-methyl-oxazol-4-ylmethoxy)-phenyl]-2-methyl-[1,3]dioxane-2-carboxylic acid

¹H NMR: 1.38 (9H, s), 1.62 (3H, s), 2.32 (3H, s), 2.71 (1H, m), 4.0 (2H, dd, J=3.8 & 11.8 Hz), 4.2 (2H, dd, J=3.7 & 11.9 Hz), 4.9 (2H, s), 6.9 (2H, d, J=8.6 Hz), 7.3 (2H, d, J=8.6 Hz).

Yield: 80.3%

### Example 106

### 2-Methyl-5-cis-{4-[2-(4-phenoxy-phenoxy)-ethoxy]-phenyl}-[1,3]dioxane-2-carboxylic acid

¹H NMR: 1.65 (3H, s), 3.22 (1H, m), 3.9 (2H, t, J=11.5 Hz), 4.1 (2H, dd, J=4.3 & 11.6 Hz), 4.29 (4H, s), 6.9 (8H, m), 7.0 (3H, t, J=8.6 Hz), 7.29 (2H, t, J=8.2 Hz).

Yield: 84.4%

### Example 107

### 2-Methyl-5-trans-{4-[2-(4-phenoxy-phenoxy)-ethoxy]-phenyl}-[1,3]dioxane-2-carboxylic acid

¹H NMR (DMSO-D₆): 1.39 (3H, s), 2.7 (1H, m), 3.9 (2H, d, J=11.4 Hz), 4.1 (2H, d, J=11.7 Hz), 4.28 (4H, s), 6.9-7.12 (9H, m), 7.36 (2H, t, J=8.0 Hz), 7.4 (2H, t, J=8.5 Hz).

Yield: 74.74%

### Example 108

### 5-cis-[4-(2-Fluoro-benzyloxy)-phenyl]-2-methyl-[1,3]dioxane-2-carboxylic acid

¹H NMR: 1.66 (3H, s), 3.2 (1H, m), 3.9 (2H, J=11.7 Hz), 4.1 (2H, dd, J=4.5 & 11.9 Hz), 5.1 (2H, s), 6.9 (2H, d, J=8.6 Hz), 7.05-7.17 (4H, m), 7.3 (1H, m), 7.45 (1H, t, J=7.4 Hz).

Yield: 93.7%

### Example 109

### 5-trans-[4-(2-Fluoro-benzyloxy)-phenyl]-2-methyl-[1,3]dioxane-2-carboxylic acid

¹H NMR: 1.68 (3H, s), 2.8 (1H, m), 4.1 (21 dd, J=3.9 & 11.4 Hz), 4.25 (2H, dd, J=3.7 & 11.9 Hz), 5.1 (2H, s), 6.99 (2H, d, J=8.6 Hz), 7.06-7.19 (2H, m), 7.30 (1H, m), 7.38 (2H, d, J=8.6 Hz), 7.5 (1H, t J=7.3 Hz).

Yield: 69.4%

### Example 110

### 5-cis-[4-(5-tert-Butyl-2-p-tolyl-2H-pyrazol-3-ylmethoxy)-phenyl]-2-methyl-[1,3]dioxane-2-carboxylic acid

¹H NMR: 1.36 (9H, s), 1.6 (3H, s), 2.3 (3H, s), 3.2 (1H, m), 3.8 (2H, t, J=11.7 Hz), 4.0 (2H, dd, J=4.62 & 11.7 Hz), 4.86 (2H, s), 6.4 (1H, s), 6.88 (2H, d, J=8.6 Hz), 7.0 (2H, d, J=8.6 Hz), 7.15 (2H, d, J=8.1 Hz), 7.4 (2H, d, J=8.2 Hz).

Yield: 63.24%

### Example 111

### 5-tram-[4-(5-tert-Butyl-2-p-tolyl-2H-pyrazol-3-ylmethoxy)-phenyl]-2-methyl-[1,33]dioxane-2-carboxylic acid

¹H NMR: 1.37 (9H, s), 1.6 (3H, s), 2.36 (3H, s), 2.8 (1H, m), 4.1 (2H, dd, J=3.8 & 11.9 Hz), 4.2 (2H, dd, J=3.8 & 11.9 Hz), 4.9 (2H, s), 6.4 (1H, s), 6.9 (2H, d, J=8.6 Hz), 7.2 (2H, d, J=8.16 Hz), 7.3 (2H, d, J=8.6 Hz), 7.4 (2H, d, J=8.2 Hz).

Yield: 51.23%

### Example 112

### 2-Methyl-5-trans-[4-(2-oxo-3-phenyl-oxazolidin-5-ylmethoxy)-phenyl]-[1,3]dioxane-2-carboxylic acid

¹H NMR (DMSO-d₆): 1.39 (3H, s), 2.7 (1H, m), 3.9 (3H, m), 4.1-4.28 (5H, m), 5.0 (1H, m), 6.9 (2H, d, J=8.4 Hz), 7.1 (1H, t, J=7.2 Hz), 7.4 (4H, m), 7.57 (2H, d, J=7.8 Hz).

Yield: 66.8%

### Example 113

### 2-Methyl-5-cis-[4-(2-oxo-3-phenyl-oxazolidin-5-ylmethoxy)-phenyl]-[1,3]dioxane-2-carboxylic acid

¹H NMR (DMSO-d₆): 1.40 (3H, s), 3.1 (1H, m), 3.7 (2H, t, J=1.6 Hz), 3.9 (3H, m), 4.2 (3H, m), 5.0 (1H, m), 6.9 (2H, d, J=8.5 Hz), 7.1 (3H, m), 7.38 (2H, t, J=8.0 Hz), 7.55 (2H, d, J=8.3 Hz).

Yield: 70.44%.

### Example 114

### 5-trans-{4-[2-(4-Methanesulfonyloxy-phenyl)-ethoxy]-phenyl}-2-methyl-[1,3]dioxane-2-carboxylic acid

¹H NMR: 1.67 (3H, s), 2.8 (1H, m), 3.08 (2H, d, J=6.7 Hz), 3.13 (3H, s), 4.10 (2H, dd, J=3.8 & 11.9 Hz), 4.14 (2H, t, J=6.7 Hz), 4.27 (2H, dd, J=3.75 & 11.9 Hz), 6.88 (2H, d, J=8.6 Hz), 7.24 (2H, d, J=8.56 Hz), 7.35 (4H, dd, J=3.4 & 8.66 Hz).

Yield: 86.6%

### Example 115

### 2-Methyl-5-trans-[4-(3-methyl-4-oxo-3,4-dihydro-quinazolin-2-ylmethoxy)-phenyl]-[1,3]dioxane-2-carboxylic acid

¹H NMR (DMSO-d₆): 1.38 (3H, s), 2.7 (1H, m), 3.67 (3H, s), 3.9 (2H, d, J=11.37 Hz), 4.1 (2H, m), 5.25 (2H, s), 7.07 (2H, d, J=8.6 Hz), 7.4 (2H, d, J=8.6 Hz), 7.55 (1H, t, J=7.6 Hz), 7.66 (1H, d, J=8.1 Hz), 7.82 (1H, t, J=7.1 Hz), 8.16 (1H, d, J=7.83 Hz).

Yield: 92.04%

### Example 116

### 5-cis-[6-(2-Fluoro-benzyloxy)-naphthalen-2-ylmethyl]-2-methyl-[1,3]dioxane-2-carboxylic acid

¹H NMR: 1.57 (3H, s), 2.40-2.53 (3H, m), 3.60 (2H, t, J=10.95 Hz), 3.96 (2H, dd, J=12.39 & 4.35 Hz), 5.24 (2H, s), 7.11-7.34 (6H, m), 7.48 (1H, s), 7.55-7.56 (1H, m), 7.69 (21H, m).

Yield: 42%

### Example 117

### 5-cis-[6-(Benzyloxy)-naphthalen-2-ylmethyl]-2-methyl-[1,3]dioxane-2-carboxylic acid

¹H NMR: 1.56 (3H, s), 2.41-2.52 (3H, m), 3.60 (2H, t, J=10.95 Hz), 3.96 (2H, dd, J=12.39 &e; 4.35 Hz), 5.17 (2H, s), 7.20-7.24 (3H, m), 7.34-7.43 (3H, m), 7.49 (3H, d, J=7.23 Hz), 7.66 (2H, dd, J=8.67 & 4.17 Hz).

Yield: 47%

### Example 118

### 2-Methyl-5-cis-{4-[2-(5-methyl-2-phenyl-oxazol-4-yl)-ethoxy]-benzyl}-[1,3]dioxane-2-carboxylic acid

¹H NMR: 1.54 (3H, s), 2.29 (3H, s), 2.37 (3H, s), 2.97 (2H, t, J=6.69 Hz), 3.51-3.55 (2H, m), 3.91 (2H, dd, J=12.57 & 4.2 Hz), 4.21 (2H, t, J=6.72 Hz), 6.82 (2H, d, J=8.55 Hz), 6.99 (2H, d, J=8.52 Hz), 7.41 (3H, m), 7.95-7.99 (2H, dd, J=7.83 & 2.88 Hz).

Yield: 32%.

### Example 119

### 5-cis-[4-(5-tert-Butyl-2-p-tolyl-2H-pyrazol-3-ylmethoxy)-benzyl]-2-methyl-[1,3]dioxane-2-carboxylic acid

¹H NMR: 1.3 (9H, s), 1.5 (3H, s), 2.3 (3H, m), 2.35 (3H, s), 3.5 (2H, t, J=10.9 Hz), 3.9 (2H, m), 4.9 (2H, s), 6.4 (1H, s), 6.8 (2H, d, J=8.5 Hz), 7.0 (2H, d, J=8.5 Hz), 7.2 (2H, d, J=8.1 Hz), 7.4 (2H, d, J=8.3 Hz).

Yield: 72%.

### Example 120

### 2-Methyl-5-cis-{4-[2-(5-methyl-2-p-tolyl-oxazol-4-yl)-ethoxy]-benzyl}-[1,3]dioxane-2-carboxylic acid

¹H NMR: 1.54 (3H, s), 2.27 (3H, s), 2.36 (3H, s), 2.38 (3H, s), 2.98 (2H, t, J=6.72 Hz), 3.52 (2H, t, J=10.74 Hz), 3.90 (2H, m), 4.20 (2H, t, J=6.66 Hz), 6.81 (2H, d, J=8.46 Hz), 6.95 (2H, d, J=8.46 Hz), 7.23 (2H, d, J=8.34 Hz), 7.85 (2H, d, J=8.13 Hz).

Yield: 32%.

### Example 121

### 2-Methyl-5-trans-{4-[2-(5-methyl-2-phenyl-oxazol-4-yl)-ethoxy]-benzyl}-[1,3]dioxane-2-carboxylic acid

### Step 1: Preparation of Methyl-2-methyl-5-trans-{4-[2-(5-methyl-2-phenyl-oxazol-4-yl)-ethoxy]-benzyl}-[1,3]dioxane-2-carboxylate

A mixture of Methyl-5-trans-(4-hydroxy-benzyl)-2-methyl-[1,3]dioxane-2-carboxylate (isolated from the mother liquor after the crystallization of cis isomer in step 2 of example 2 above) (750 mg), 12-(5-methyl-2-phenyl-oxazol-4-yl)-ethyl methane sulfonate (790 mg) and potassium carbonate (780 mg) in anhydrous dimethyl formamide (10 mL) was stirred at 80° C. for extended periods in an inert atmosphere. Reaction mixture was cooled to ambient temperature, poured into ice cold water and extracted with ethyl acetate. The combined organic extract was washed with water, brine solution, dried over sodium sulphate and evaporated under reduced pressure. Crude product was flash chromatographed over silica gel using a mixture of ethyl acetate and petroleum ether as eluent to obtain 971 mg of pure product.

### Step 2: Preparation of 2-Methyl-5-trans-{4-[2-(5-methyl-2-phenyl-oxazol-4-yl)-ethoxy]-benzyl}-[1,3]dioxane-2-carboxylic acid

To a solution of Methyl-2-methyl-5-trans-{4-[2-(5-methyl-2-phenyl-oxazol-4-yl)-ethoxy]-benzyl}-[1,3]dioxane-2-carboxylate (263 mg) in methanol was added another solution of sodium hydroxide (46.5 mg) in water and the reaction mixture was stirred at ambient temperature for 15 hours. Solvents were evaporated and the residue was dissolved in water, acidified with 1N HCl and extracted with ethyl acetate. The combined organic extract was washed with water, brine, dried over sodium sulphate and evaporated under reduced pressure. Crude product was flash chromatographed over silica gel using 2% methanol in chloroform as eluent to obtain 160 mg of pure product.

¹H NMR: 1.61 (3H, s), 2.38 (4H, s), 2.87 (2H, d, J=7.8 Hz), 3.01 (2H, t, J=6.90 Hz), 3.72 (2H, d, J=10.95 Hz), 3.92 (2H, d, J=9.51 Hz), 4.21 (2H, t, J=6.99 Hz), 6.82 (2H, d, J=8.55 Hz), 7.06 (2H, d, J=8.511 Hz), 7.41-7.45 (3H, m), 7.96 (2H, dd, J=4.35 & 7.74 Hz).

Yield: 52%

The following compounds were prepared by procedure similar to those described in example 121 with appropriate variations of reactants, reaction conditions and quantities of reagents.

### Example 122

### 2-Methyl-5-trans-[4-(5-methyl-2-phenyl-oxazol-4-ylmethoxy)-benzyl]-[1,3]dioxane-2-carboxylic acid

### Example 123

### 2-Methyl-5-trans-[4-(5-pyridin-2-yl-pyrrolidin-2-ylmethoxy)-benzyl]-[1,3]dioxane-2-carboxylic acid

### Example 124

### 2-Methyl-5-trans-{4-[2-(methyl-pyridin-2-yl-amino)-ethoxy]-benzyl}-[1,3]dioxane-2-carboxylic acid

### Example 126

### 2-Methyl-5-trans-[4-(5-methyl-2-p-tolyl-oxazol-4-ylmethoxy)-benzyl]-[1,3]dioxane-2-carboxylic acid

### Example 127

### 5-trans-[4-(2-Carbazol-9-yl-ethoxy)-benzyl]-2-methyl-[1,3]dioxane-2-carboxylic acid

### Example 128

### 5-trans-[4-(2-Indol-1-yl-ethoxy)-benzyl]-2-methyl-[1,3]dioxane-2-carboxylic acid

### Example 129

### 2-Methyl-5-trans-{4-[5-methyl-2-(5-methyl-thiophen-2-yl)-oxazol-4-ylmethoxy]-benzyl}-[1,3]dioxane-2-carboxylic acid

### Example 130

### 15-trans-{4-[2-(2,3-Dihydro-benzo[1,4]thiazin-4-yl)-ethoxy]-benzyl}-2-methyl-[1,3]dioxane-2-carboxylic acid

### Example 131

### 2-Methyl-5-trans-[4-(2-phenothiazin-10-yl-ethoxy)-benzyl]-[1,3]dioxane-2-carboxylic acid

### Example 132

### 5-trans-{4-[2-(4-Hexyl-3-oxo-3,4-dihydro-2H-benzo[1,4]oxazin-2-yl)-ethoxy]-benzyl}-2-methyl-[1,3]dioxane-2-carboxylic acid

### Example 133

### 2-Methyl-5-trans-(4-{2-[5-methyl-2-(5-methyl-thiophen-2-yl)-oxazol-4-yl]-ethoxy}-benzyl)-[1,3]dioxane-2-carboxylic acid

### Example 134

### 2-Methyl-5-trans-{4-[2-(5-methyl-2-phenyl-oxazol-4-yl)-ethoxy]-benzyl}-[1,3]dioxane-2-carboxylic acid

### Example 135

### 2-Methyl-5-trans-{4-[2-(5-methyl-2-phenyl-oxazol-4-yl)-ethoxy]-benzyl}-[1,3]dioxane-2-carboxylic acid

### Example 136

### 2-Methyl-5-trans-{4-[3-(4-phenoxy-phenoxy)-propoxy]-benzyl}-[1,3]dioxane-2-carboxylic acid

### Example 137

### 5-trans-{4-[2-(4-Methanesulfonyloxy-phenyl)-ethoxy]-benzyl}-2-methyl-[1,3]dioxane-2-carboxylic acid

### Example 138

### 5-trans-[4-(2-tert-Butyl-5-methyl-oxazol-4-ylmethoxy)-benzyl]-2-methyl-[1,3]dioxane-2-carboxylic acid

### Example 139

### 2-Methyl-5-trans-{4-[4-methyl-2-(4-trifluoromethyl-phenyl)-thiazol-5-ylmethoxy]-benzyl}-[1,3]dioxane-2-carboxylic acid

### Example 140

### 2-Methyl-5-trans-{4-[2-(4-phenoxy-phenoxy)-ethoxy]-benzyl}-[1,3]dioxane-2-carboxylic acid

### Example 141

### 5-trans-[4-(2-Fluoro-benzyloxy)-benzyl]-2-methyl-[1,3]dioxane-2-carboxylic acid

### Example 142

### 2-Methyl-5-trans-(4-{2-[2-methyl-5-(4-methylsulfanyl-phenyl)-pyrrol-1-yl]-ethoxy}-benzyl)-[1,3]dioxane-2-carboxylic acid

### Example 144

### 5-trans-{4-[2-(2-tert-Butyl-5-methyl-oxazol-4-yl)-ethoxy]-benzyl}-2-methyl-[1,3]dioxane-2-carboxylic acid

### Example 145

### 5-trans-[6-(2-Fluoro-benzyloxy)-naphthalen-2-ylmethyl]-2-methyl-[1,3]dioxane-2-carboxylic acid

### Example 146

### 5-trans-[6-(Benzyloxy)-naphthalen-2-ylmethyl]-2-methyl-[1,3]dioxane-2-carboxylic acid

### Example 147

### (Z)-2-Methyl-5-trans-{4-[2-(phenyl-pyridin-2-yl-methyleneaminooxy)-ethoxy]-benzyl}-[1,3]dioxane-2-carboxylic acid

### Example 149

### (2)-2-Methyl-5-trans-{4-[2-(phenyl-pyridin-2-yl-methyleneaminooxy)-ethoxy]-benzyl}-[1,3]dioxane-2-carboxylic acid

### Example 150

### 5-trans-[4-(5-tert-Butyl-2-p-tolyl-2H-pyrazol-3-ylmethoxy)-benzyl]-2-methyl-[1,3]dioxane-2-carboxylic acid

### Example 151

### 2-Methyl-5-trans-{4-[2-(5-methyl-2-p-tolyl-oxazol-4-yl)-ethoxy]-benzyl}-[1,3]dioxane-2-carboxylic acid

### Preparation of Salts

Sodium and potassium sales of the compounds described above were prepared by following the general procedure described below.

To a solution of carboxylic acid derivatives of the novel compounds (1 mmol) in alcoholic solvent like methanol, ethanol, propanol, isopropanol, butanol, t-butanol and the like was added a solution of sodium or potassium alkoxide (0.95 mmol) in alcoholic solvent and the reaction mixture was stirred for 3 hours at 25-30° C. Solvent was evaporated and the residue was triturated with dry diethyl ether or diisopropyl ether to obtain the salt of the corresponding carboxylic acid.

The compounds of the present invention lowered triglyceride, total cholesterol, LDL, VLDL and increased HDL and lowered serum glucose levels. This was demonstrated by in vitro as well as in vivo animal experiments.

### A) Demonstration of In Vitro Efficacy of Compounds:

In vitro hPPAR α & hPPARγ activities were determined as per in-house protocols and the results of representative compounds are provided below as a proof of the efficacies of the novel class of compounds disclosed above.

| Example No. | EC₅₀ (PPAR alpha) | EC₅₀ (PPAR gamma) |
|---|---|---|
| 61 | 1.09 μM | 0.096 μM |
| 66 | 0.072 μM | 0.015 μM |
| 70 | 0.6 μM | 0.0198 μM |

### B) Demonstration of In Vivo Efficacy of Compounds:

i) Serum Triglyceride and Total Cholesterol Lowering Activity in Swiss Albino Mice:

Male Swiss albino mice (SAM) were bred in Zydus animal house. All these animals were maintained under 12 hour light and dark cycle at 25±1° C. Animals were given standard laboratory chow (NIN, Hyderabad, India) and water ad libitum. SAM of 20-30 g body weight range was used. The protocol approved by Institutional Animal Ethics Committee is being used.

The test compounds were administered orally to Swiss albino mice at 0.001 to 50 mg/kg/day dose for 6 days. The compound was administered after suspending it in 0.25% CMC or dissolving it in water, when compound is water-soluble. Control mice were treated with vehicle (0.25% of Carboxymethyl cellulose; dose 10 ml/kg).

The blood samples were collected on 0^{th} day and in fed state 1 hour after drug administration on 6^{th} day of the treatment. The blood was collected in non heparinised capillary and the serum was analyzed for triglyceride and total cholesterol (Wieland, O. Methods of Enzymatic analysis. Bergermeyer, H., O., Ed., 1963. 211-214; Trinder, P. Ann. Clin. Biochem. 1969. 6: 24-27). Measurement of serum triglyceride and total cholesterol was done using commercial kits (Zydus-Cadila, Pathline, Ahmedabad, India).

Formula for Calculation:

Percentage reduction in triglycerides/total cholesterol were calculated according to the formula:

$$Percentage reduction ( \% ) = 1 - [ \frac{TT / OT}{TC / OC} ] \times 100$$

OC=Zero day control group value OT=Zero day treated group value
TC=Test day control group TT=Test day treated group

TABLE 1
Triglyceride lowering activity in Swiss albino mice:

| | Dose | |
|---|---|---|
| Example No. | (mg/kg/day) | % Triglyceride lowering |
| 61 | 10 | 23 |
| 66 | 10 | 40 |
| 100 | 10 | 44 |

ii) Serum triglyceride and total cholesterol lowering activity in Hamster of Syrian golden stain:

Male and Female Hamster of Syrian golden stain were bred in Zydus animal house. All these animals were maintained under 12-hour light and dark cycle at 22±3 degree C. The protocol approved by Institutional Animal Ethics Committee is being used. Animals of 3-12 weeks age (80-150 gm body weight) were taken for study. Near the end of the acclimatization period, animals judged to be suitable for testing will bee weighed. Six animals will be selected for normal NIN (NIN, Hyderabad, India) diet average bodyweight was not significantly different from the rest of the animals. Other animals were put on HF-HC (High fat and high cholesterol) diet for 14 days. On day 14 all the HF-HC diet fed animals were selected which had gained their body weight significantly more than the normal diet group animals. The selected animals were divided into different groups in such a way that the average bodyweight of the animals in each group was not significantly different from the other groups.

Each animal received a single dose of ZY compounds at 0.001 to 50 mg/kg/ as a carbomethoxy cellulose or polyethylene glycol in the evening administered as an oral gavage daily for 15 days. On day 14, after 1 hr of dose administration, non-fasted blood samples were collected in non heparinized capillary from animals for determination of total cholesterol (TC), triglyceride (TG) (Wieland, O. Methods of Enzymatic analysis. Bergermeyer, H., O., Ed., 1963. 211-214; Trinder, P. Ann. Clin. Biochem. 1969. 6: 24-27). Measurement of serum triglyceride and total cholesterol was done using commercial kits (Pointe Scientific.Inc.USA.) And on day 14 night all animals will be kept for fasting for 12-16 hours. On day 15, after 1 hour of dose administration fasted blood samples will be collected from animals for determination of high density lipoprotein (HDL), and low density lipoprotein (LDL) in serum. The animals will then be euthanized by carbon-dioxide asphyxia (and cervical dislocation, if necessary) and heart, kidney and liver will be resected and weighed.

### Formula for Calculation:

Percentage reduction in triglycerides/total cholesterol were calculated according to the formula:

Percentage reduction(%)=(TT−TC)/TC*100

TC=Test day control group TT=Test day treated group.

TABLE 2

| | Dose | |
|---|---|---|
| Example No. | (mg/kg/day) | % Triglyceride lowering |
| 61 | 3 | 30 |
| 96 | 3 | 41 |
| 106 | 3 | 50 |

The compounds of the present invention improves insulin sensitivity, impaired glucose tolerance and reduced serum glucose levels, TG, FFA and cholesterol in db/db, ob/ob mice and zucker fa/fa rats.

No adverse effects were observed for any of the mentioned compounds of invention. The compounds of the present invention showed good serum glucose, lipid and cholesterol lowering activity in the experimental animals used. These compounds are useful for the testing/prophylaxis of diseases caused by hyperlipidemia, hypercholesterolemia, hyperinsulinemia, hyperglycemia such as NIDDM, cardiovascular diseases, stroke, hypertension, obesity since such diseases are interlinked to each other.

1. Compounds of the general formula (I), 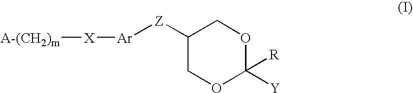
their tautomeric forms, their stereoisomers, their pharmaceutically acceptable salts, wherein ‘A’ represents an optionally substituted single or fused group selected from aryl, heteroaryl, heterocyclyl groups; or groups represented by 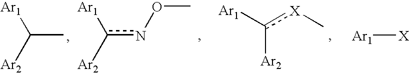
wherein ‘Ar’, is selected from optionally substituted phenyl or naphthyl group ‘Ar₁’ and ‘Ar₂’ may be the same or different and independently represents an optionally substituted single or fused aryl, heteroaryl or a heterocyclic group; ‘X’ represents oxygen, sulfur or nitrogen; ‘Y’ represents COOR¹, CONR¹R²; Z represents a bond or —CH₂—; ‘m’ is an integer from 1-3; R¹, R² & R² may be same or different and independently represents hydrogen, optionally substituted groups selected from linear or branched alkyl or aryl groups.

2. A compound as claimed in claim 1, wherein the alkyl group is preferably selected from a linear or branched alkyl group comprising from one to eight carbon atoms.

3. A compound as claimed in claim 1, wherein the aryl group is preferably selected from monocyclic, bicyclic or tricyclic aryl group.

4. A compound as claimed in claim 1 wherein the aryl group is more preferably selected from phenyl, naphthyl, tetrahydronaphthyl, indane, biphenyl groups.

5. A compound as claimed in claim 1 wherein the heterocyclyl is selected from saturated, partially saturated or unsaturated aromatic or non-aromatic mono, bi or tricyclic groups, containing one or more heteroatoms selected from N, O, S.

6. A compound as claimed in claim 1 wherein the heterocyclyl group is more preferably selected from aziridinyl, azetidinyl, pyrrolidinyl, imidazolidinyl, piperidinyl, piperazinyl, 2-oxopiperidinyl, 4-oxopiperidinyl, 2-oxopiperazinyl, 3-oxopiperazinyl, morpholinyl, thiomorpholinyl, 2-oxomorpholinyl, azepinyl, diazepinyl, oxapinyl, thiazepinyl, oxazolidinyl, thiazolidinyl, dihydrothiophene, dihydropyran, dihydrofuran, dihydrothiazole, benzopyranyl, benzopyranonyl, benzodihydrofuranyl, benzodihydrothienyl, pyrazolopyrimidonyl, azaquinazolinoyl, thienopyrimidonyl, quinazolonyl, pyrimidonyl, benzoxazinyl, benzoxazinonyl, benzothiazinyl, benzothiazinonyl, thieno piperidinyl groups.

7. A compound as claimed in claim 1 wherein the heteroaryl groups is selected from optionally fused mono, bi or tricyclic aromatic heterocyclic groups comprising from one or more hetero atoms selected from O, N or S.

8. A compound as claimed in claim 1 wherein the heteroaryl group is more preferably selected from pyridyl, thienyl, furyl, pyrrolyl, oxazolyl, thiazolyl, isothiazolyl, imidazolyl, isoxazolyl, oxadiazolyl, thiadiazolyl, triazolyl, tetrazolyl, benzofuranyl, benzothienyl, indolinyl, indolyl, azaindolyl, azaindolinyl, pyrazolopyrimidinyl, azaquinazolinyl, pyridofuranyl, pyridothienyl, thienopyrimidyl, quinolinyl, pyrimidinyl, pyrazolyl, quinazolinyl, pyridazinyl, triazinyl, benzimidazolyl, benzotriazolyl, phthalazynil, naphthylidinyl, purinyl, carbazolyl, phenothiazinyl, phenoxazinyl, benzoxazolyl, benzothiazolyl groups.

9. A compound as claimed in claim 1, wherein when any one of ‘A’, ‘Ar₁’ or ‘Ar₂’ is substituted, the substituents may be selected from hydroxy, oxo, halo, thio, nitro, amino, cyano, formyl, or optionally substituted groups selected from amidino, hydrazino, alkyl, haloalkyl, perhaloalkyl, alkoxy, haloalkoxy, perhaloalkoxy, alkenyl, alkynyl, cycloalkyl, cycloalkenyl, bicycloalkyl, bicycloalkenyl, alkoxy, alkenoxy, cycloalkoxy, aryl, aryloxy, aralkyl, aralkoxy, heterocyclyl, heteroaryl, heterocyclylalkyl, heteroaralkyl, heteroaryloxy, heteroaralkoxy, heterocyclyloxy, heterocyclylalkoxy, heterocyclylalkoxyacyl, acyl, acyloxy, acylamino, monosubstituted or disubstituted amino, arylamino, aralkylamino, carboxylic acid and its derivatives such as esters and amides, carbonylamino, hydroxyalkyl, aminoalkyl, alkoxyalkyl, aryloxyalkyl, aralkoxyalkyl, alkylthio, thioalkyl, arylthio, alkylsulfonylamino, alkylsulfonyloxy, alkoxycarbonylamino, aryloxycarbonylamino, aralkyloxycarbonylamino, aminocarbonylamino, alkylaminocarbonylamino, alkoxyamino, hydroxylamino, sulfenyl derivatives, sulfonyl derivatives, sulfonic acid and its derivatives.

10. A compound as claimed in claim 1 wherein the substituents on any of ‘A’, ‘Ar₁’ or ‘Ar₂’ are more preferably selected from hydroxy, halo, oxo, or optionally substituted groups selected from alkyl, monosubstituted or disubstituted amino, alkoxy, acyl, aryl, aryloxy, aralkyl, aralkoxy, heterocyclyl, heteroaryl, heterocyclylalkyl, heteroaralkoxy, heterocyclyloxy, alkylthio, arylthio, alkylsulfonylamino, alkylsulfonyloxy, carboxylic acid and its derivatives such as esters and amides group.

11. A compound as claimed in claim 1 wherein the substituents on ‘Ar’ are selected from halogen, optionally substituted groups selected from linear or branched alkyl, alkoxy, thioalkyl, haloalkyl, haloalkoxy, acyl, arylaminoalkyl, aminoalkyl groups.

12. A compound as claimed in claim 1 preferably selected from
Methyl-2-methyl-5-{4-[2-(5-methyl-2-p-tolyl-oxazol-4-yl)-ethoxy]-benzyl}-[1,3]dioxane-2-carboxylate;
Methyl-5-[6-(2-fluoro-benzyloxy)-naphthalen-2-ylmethyl]-2-methyl-[1,3]dioxane-2-carboxylate:
Methyl-5-[6-(benzyloxy)-naphthalen-2-ylmethyl]-2-methyl-[1,3]dioxane-2-carboxylate:
Methyl-2-methyl-5-cis-{4-[2-(5-methyl-2-phenyl-oxazol-4-yl)-ethoxy]-benzyl}-[1,3]dioxane-2-carboxylate;
Methyl-2-methyl-5-{4-[2-(5-methyl-2-phenyl-oxazol-4-yl)-ethoxy]-benzyl}-[1,3]dioxane-2-carboxylate;
Methyl-2-methyl-5-cis-[4-(5-methyl-2-phenyl-oxazol-4-ylmethoxy)-benzyl]-[1,3]dioxane-2-carboxylate;
Methyl-2-methyl-5-cis-[4-(1-pyridin-2-yl-pyrrolidin-2-ylmethoxy)-benzyl]-[1,3]dioxane-2-carboxylate;
Methyl-2-methyl-5-cis-{4-[2-(methyl-pyridin-2-yl-amino)-ethoxy]-benzyl}-[1,3]dioxane-2-carboxylate;
Methyl-2-methyl-5-[4-(3-methyl-4-oxo-3,4-dihydro-quinazolin-2-ylmethoxy)-benzyl]-[1,3]dioxane-2-carboxylate;
Methyl-2-methyl-5-cis-[4-(5-methyl-2-p-tolyl-oxazol-4-ylmethoxy)-benzyl]-[1,3]dioxane-2-carboxylate;
Methyl-5-cis-[4-(2-carbazol-9-yl-ethoxy)-benzyl]-2-methyl-[1,3]dioxane-2-carboxylate;
Methyl-5-cis-[4-(2-indol-1-yl-ethoxy)-benzyl]-2-methyl-[1,3]dioxane-2-carboxylate;
Methyl-5-{4-[2-(2,3-dihydro-benzo[1,4]oxazin-4-yl)-ethoxy]-benzyl}-2-methyl-[1,3]dioxane-2-carboxylate;
Methyl-2-methyl-5-cis-{4-[5-methyl-2-(5-methyl-thiophen-2-yl)-oxazol-4-ylmethoxy]-benzyl}-[1,3]dioxane-2-carboxylate;
Methyl-2-methyl-5-cis-(4-{2-[5-methyl-2-(5-methyl-thiophen-2-yl)-oxazol-4-yl]-ethoxy}-benzyl)-[1,3]dioxane-2-carboxylate;
Methyl-2-methyl-5-cis-{4-[3-(4-phenoxy-phenoxy)-propoxy]-benzyl}-[1,3]dioxane-2-carboxylate;
Methyl-5-cis-{4-[2-(4-methanesulfonyloxy-phenyl)-ethoxy]-benzyl}-2-methyl-[1,3]dioxane-2-carboxylate;
Methyl-5-cis-[4-(2-tert-Butyl-5-methyl-oxazol-4-ylmethoxy)-benzyl]-2-methyl-[1,3]dioxane-2-carboxylate;
Methyl-2-methyl-5-cis-{4-[4-methyl-2-(4-trifluoromethyl-phenyl)-thiazol-5-ylmethoxy]-benzyl}-[1,3]dioxane-2-carboxylate;
Methyl-2-methyl-5-cis-{4-[2-(4-phenoxy-phenoxy)-ethoxy]-benzyl}-[1,3]dioxane-2-carboxylate;
Methyl-5-cis-[4-(2-fluoro-benzyloxy)-benzyl]-2-methyl-[1,3]dioxane-2-carboxylate;
Methyl-2-methyl-5-[4-(2-phenoxazin-10-yl-ethoxy)-benzyl]-[1,3]dioxane-2-carboxylate;
Methyl-2-methyl-5-cis-[4-(2-oxo-3-phenyl-oxazolidin-5-ylmethoxy)-benzyl]-[1,3]dioxane-2-carboxylate;
Methyl-5-cis-{4-[2-(2,3-dihydro-benzo[1,4]thiazin-4-yl)-ethoxy]-benzyl}-2-methyl-[1,3]dioxane-2-carboxylate;
Methyl-2-methyl-5-cis-[4-(2-phenothiazin-10-yl-ethoxy)-benzyl]-[1,3]dioxane-2-carboxylate;
Methyl-5-cis-{4-[2-(4-hexyl-3-oxo-3,4-dihydro-2H-benzo[1,4]oxazin-2-yl)-ethoxy]-benzyl}-2-methyl-[1,3]dioxane-2-carboxylate;
Methyl-2-methyl-5-cis-(4-{2-[2-methyl-5-(4-methylsulfanyl-phenyl)-pyrrol-1-yl]-ethoxy}-benzyl)-[1,3]dioxane-2-carboxylate;
Methyl-5-cis-{4-[2-(2-tert-Butyl-5-methyl-oxazol-4-yl)-ethoxy]-benzyl}-2-methyl-[1,3]dioxane-2-carboxylate;
(Z)-Methyl-2-methyl-5-cis-{4-[2-(phenyl-pyridin-2-yl-methyleneaminooxy)-ethoxy]-benzyl}-[1,3]dioxane-2-carboxylate;
(E)-Methyl-2-methyl-5-cis-{4-[2-(phenyl-pyridin-2-yl-methyleneaminooxy)-ethoxy]-benzyl}-[1,3]dioxane-2-carboxylate;
Methyl-2-methyl-5-{4-[2-(5-methyl-2-p-tolyl-oxazol-4-yl)-ethoxy]-benzyl}-[1,3]dioxane-2-carboxylate;
Methyl-5-cis-[4-(5-tert-Butyl-2-p-tolyl-2H-pyrazol-3-ylmethoxy)-benzyl]-2-methyl-[1,3]dioxane-2-carboxylate;
Methyl-2-methyl-5-trans-[4-(5-methyl-2-phenyl-oxazol-4-ylmethoxy)-benzyl]-[1,3]dioxane-2-carboxylate;
Methyl-2-methyl-5-trans-[4-(1-pyridin-2-yl-pyrrolidin-2-ylmethoxy)-benzyl]-[1,3]dioxane-2-carboxylate;
Methyl-2-methyl-5-trans-{4-[2-(methyl-pyridin-2-yl-amino)-ethoxy]-benzyl}-[1,3]dioxane-2-carboxylate;
Methyl-2-methyl-5-trans-[4-(5-methyl-2-p-tolyl-oxazol-4-ylmethoxy)-benzyl]-[1,3]dioxane-2-carboxylate;
Methyl-5-trans-[4-(2-carbazol-9-yl-ethoxy)-benzyl]-2-methyl-[1,3]dioxane-2-carboxylate;
Methyl-5-trans-[4-(2-indol-1-yl-ethoxy)-benzyl]-2-methyl-[1,3]dioxane-2-carboxylate;
Methyl-2-methyl-5-trans-{4-[5-methyl-2-(5-methyl-thiophen-2-yl)-oxazol-4-ylmethoxy]-benzyl}-[1,3]dioxane-2-carboxylate;
Methyl-2-methyl-5-trans-(4-{2-[5-methyl-2-(5-methyl-thiophen-2-yl)-oxazol-4-yl]-ethoxy}-benzyl)-[1,3]dioxane-2-carboxylate;
Methyl-2-methyl-5-trans-{4-[3-(4-phenoxy-phenoxy)-propoxy]-benzyl}-[1,3]dioxane-2-carboxylate;
Methyl-5-trans-{4-[2-(4-methanesulfonyloxy-phenyl)-ethoxy]-benzyl}-2-methyl-[1,3]dioxane-2-carboxylate;
Methyl-5-trans-[4-(2-tert-Butyl-5-methyl-oxazol-4-ylmethoxy)-benzyl]-2-methyl-[1,3]dioxane-2-carboxylate;
Methyl-2-methyl-5-trans-{4-[4-methyl-2-(4-trifluoromethyl-phenyl)-thiazol-5-ylmethoxy]-benzyl}-[1,3]dioxane-2-carboxylate;
Methyl-2-methyl-5-trans-{4-[2-(4-phenoxy-phenoxy)-ethoxy]-benzyl}-[1,3]dioxane-2-carboxylate;
Methyl-5-trans-[4-(2-fluoro-benzyloxy)-benzyl]-2-methyl-[1,3]dioxane-2-carboxylate;
Methyl-2-methyl-5-trans-[4-(2-oxo-3-phenyl-oxazolidin-5-ylmethoxy)-benzyl]-[1,3]dioxane-2-carboxylate;
Methyl-5-trans-{4-[2-(2,3-dihydro-benzo[1,4]thiazin-4-yl)-ethoxy]-benzyl}-2-methyl-[1,3]dioxane-2-carboxylate;
Methyl-2-methyl-5-trans-[4-(2-phenothiazin-10-yl-ethoxy)-benzyl]-[1,3]dioxane-2-carboxylate;
Methyl-5-trans-{4-[2-(4-hexyl-3-oxo-3,4-dihydro-2H-benzo[1,4]oxazin-2-yl)-ethoxy]-benzyl}-2-methyl-[1,3]dioxane-2-carboxylate;
Methyl-2-methyl-5-trans-(4-{2-[2-methyl-5-(4-methylsulfanyl-phenyl)-pyrrol-1-yl]-ethoxy}-benzyl)-[1,3]dioxane-2-carboxylate;
Methyl-5-trans-{4-[2-(2-tert-Butyl-5-methyl-oxazol-4-yl)-ethoxy]-benzyl}-2-methyl-[1,3]dioxane-2-carboxylate;
(Z)-Methyl-2-methyl-5-trans-{4-[2-(phenyl-pyridin-2-yl-methyleneaminooxy)-ethoxy]-benzyl}-[1,3]dioxane-2-carboxylate;
(E)-Methyl-2-methyl-5-trans-{4-[2-(phenyl-pyridin-2-yl-methyleneaminooxy)-ethoxy]-benzyl}-[1,3]dioxane-2-carboxylate;
Methyl-5-trans-[4-(5-tert-Butyl-2-p-tolyl-2H-pyrazol-3-ylmethoxy)-benzyl]-2-methyl-[1,3]dioxane-2-carboxylate;
Methyl-2-methyl-5-cis-[4-(5-methyl-2-phenyl-oxazol-4-ylmethoxy)-phenyl]-[1,3]dioxane-2-carboxylate;
Methyl-2-methyl-5-trans-[4-(5-methyl-2-phenyl-oxazol-4-ylmethoxy)-phenyl]-[1,3]dioxane-2-carboxylate;
Methyl-2-methyl-5-cis-{4-[2-(5-methyl-2-phenyl-oxazol-4-yl)-ethoxy]-phenyl}-[1,3]dioxane-2-carboxylate;
Methyl-2-methyl-5-cis-[4-(2-phenoxazin-10-yl-ethoxy)-phenyl]-[1,3]dioxane-2-carboxylate;
Methyl-2-methyl-5-cis-[4-(3-methyl-4-oxo-3,4-dihydro-quinazolin-2-ylmethoxy)-phenyl]-[1,3]dioxane-2-carboxylate;
Methyl-5-cis-[4-(2-indol-1-yl-ethoxy)-phenyl]-2-methyl-[1,3]dioxane-2-carboxylate;
Methyl-5-cis-{4-[2-(5-ethyl-pyridin-2-yl)-ethoxy]-phenyl}-2-methyl-[1,3]dioxane-2-carboxylate;
Methyl-2-methyl-5-cis-[4-(5-methyl-2-p-tolyl-oxazol-4-ylmethoxy)-phenyl]-[1,3]dioxane-2-carboxylate;
Methyl-2-Methyl-5-cis-{4-[2-(5-methyl-2-p-tolyl-oxazol-4-yl)-ethoxy]-phenyl}-[1,3]dioxane-2-carboxylate;
Methyl-5-cis-{4-[2-(4-methanesulfonyloxy-phenyl)-ethoxy]-phenyl}-2-methyl-[1,3]dioxane-2-carboxylate;
Methyl-2-methyl-5-trans-[4-(5-methyl-2-p-tolyl-oxazol-4-ylmethoxy)-phenyl]-[1,3]dioxane-2-carboxylate;
Methyl-2-methyl-5-trans-{4-[2-(5-methyl-2-phenyl-oxazol-4-yl)-ethoxy]-phenyl}-[1,3]dioxane-2-carboxylate;
Methyl-2-methyl-5-trans-{4-[2-(5-methyl-2-p-tolyl-oxazol-4-yl)-ethoxy]-phenyl}-[1,3]dioxane-2-carboxylate;
Methyl-2-methyl-5-trans-{4-[4-methyl-2-(4-trifluoromethyl-phenyl)-thiazol-5-ylmethoxy]-phenyl}-[1,3]dioxane-2-carboxylate;
Methyl-2-methyl-5-cis-{4-[4-methyl-2-(4-trifluoromethyl-phenyl)-thiazol-5-ylmethoxy]-phenyl}-[1,3]dioxane-2-carboxylate;
Methyl-2-methyl-5-cis-(4-{2-[2-methyl-5-(4-methylsulfanyl-phenyl)-pyrrol-1-yl]-ethoxy}-phenyl)-[1,3]dioxane-2-carboxylate;
Methyl-5-cis-[4-(2-tert-Butyl-5-methyl-oxazol-4-ylmethoxy)-phenyl]-2-methyl-[1,3]dioxane-2-carboxylate;
Methyl-5-trans-[4-(2-tert-Butyl-5-methyl-oxazol-4-ylmethoxy)-phenyl]-2-methyl-[1,3]dioxane-2-carboxylate;
Methyl-2-methyl-5-cis-{4-[2-(4-phenoxy-phenoxy)-ethoxy]-phenyl}-[1,3]dioxane-2-carboxylate;
Methyl-2-methyl-5-trans-{4-[2-(4-phenoxy-phenoxy)-ethoxy]-phenyl}-[1,3]dioxane-2-carboxylate;
Methyl-5-cis-[4-(2-fluoro-benzyloxy)-phenyl]-2-methyl-[1,3]dioxane-2-carboxylate;
Methyl-5-trans-[4-(2-fluoro-benzyloxy)-phenyl]-2-methyl-[1,3]dioxane-2-carboxylate;
Methyl-5-cis-[4-(5-tert-Butyl-2-p-tolyl-2H-pyrazol-3-ylmethoxy)-phenyl]-2-methyl-[1,3]dioxane-2-carboxylate;
Methyl-5-trans-[4-(5-tert-Butyl-2-p-tolyl-2H-pyrazol-3-ylmethoxy)-phenyl]-2-methyl-[1,3]dioxane-2-carboxylate;
Methyl-2-methyl-5-trans-[4-(2-oxo-3-phenyl-oxazolidin-5-ylmethoxy)-phenyl]-[1,3]dioxane-2-carboxylate;
Methyl-2-methyl-5-cis-[4-(2-oxo-3-phenyl-oxazolidin-5-ylmethoxy)-phenyl]-[1,3]dioxane-2-carboxylate;
Methyl-5-trans-{4-[2-(4-Methanesulfonyloxy-phenyl)-ethoxy]-phenyl}-2-methyl-[1,3]dioxane-2-carboxylate;
Methyl-2-Methyl-5-trans-[4-(3-methyl-4-oxo-3,4-dihydro-quinazolin-2-ylmethoxy)-phenyl]-[1,3]dioxane-2 carboxylate;
2-methyl-5-{4-[2-(5-methyl-2-phenyl-oxazol-4-yl)-ethoxy]-benzyl}-[1,3]dioxane-2-carboxylic acid and its pharmaceutically acceptable salts;
2-Methyl-5-{4-[2-(5-methyl-2-p-tolyl-oxazol-4-yl)-ethoxy]-benzyl}-[1,3]dioxane-2-carboxylic acid and its pharmaceutically acceptable salts;
2-Methyl-5-cis-[4-(5-methyl-2-phenyl-oxazol-4-ylmethoxy)-benzyl]-[1,3]dioxane-2-carboxylic acid and its pharmaceutically acceptable salts;
2-Methyl-5-cis-[4-(1-pyridin-2-yl-pyrrolidin-2-ylmethoxy)-benzyl]-[1,3]dioxane-2-carboxylic acid and its pharmaceutically acceptable salts;
2-Methyl-5-cis-{4-[2-(methyl-pyridin-2-yl-amino)-ethoxy]-benzyl}-[1,3]dioxane-2-carboxylic acid and its pharmaceutically acceptable salts;
2-Methyl-5-[4-(3-methyl-4-oxo-3,4-dihydro-quinazolin-2-ylmethoxy)-benzyl]-[1,3]dioxane-2-carboxylic acid and its pharmaceutically acceptable salts;
2-Methyl-5-[4-(2-phenoxazin-10-yl-ethoxy)-benzyl]-[1,3]dioxane-2-carboxylic acid and its pharmaceutically acceptable salts;
2-Methyl-5-cis-[4-(5-methyl-2-p-tolyl-oxazol-4-ylmethoxy)-benzyl]-[1,3]dioxane-2-carboxylic acid and its pharmaceutically acceptable salts;
5-cis-[4-(2-Carbazol-9-yl-ethoxy)-benzyl]-2-methyl-[1,3]dioxane-2-carboxylic acid and its pharmaceutically acceptable salts;
5-cis-[4-(2-Indol-1-yl-ethoxy)-benzyl]-2-methyl-[1,3]dioxane-2-carboxylic acid and its pharmaceutically acceptable salts;
5-{4-[2-(2,3-Dihydro-benzo[1,4]oxazin-4-yl)-ethoxy]-benzyl}-2-methyl-[1,3]dioxane-2-carboxylic acid and its pharmaceutically acceptable salts;
2-Methyl-5-cis-{4-[5-methyl-2-(5-methyl-thiophen-2-yl)-oxazol-4-ylmethoxy]-benzyl}-[1,3]dioxane-2-carboxylic acid and its pharmaceutically acceptable salts;
5-cis-{4-[2-(2,3-Dihydro-benzo[1,4]thiazin-4-yl)-ethoxy]-benzyl}-2-methyl-[1,3]dioxane-2-carboxylic acid and its pharmaceutically acceptable salts;
2-Methyl-5-cis-[4-(2-phenothiazin-10-yl-ethoxy)-benzyl]-[1,3]dioxane-2-carboxylic acid and its pharmaceutically acceptable salts;
5-cis-{4-[2-(4-Hexyl-3-oxo-3,4-dihydro-2H-benzo[1,4]oxazin-2-yl)-ethoxy]-benzyl}-2-methyl-[1,3]dioxane-2-carboxylic acid and its pharmaceutically acceptable salts;
2-Methyl-5-cis-(4-{2-[5-methyl-2-(5-methyl-thiophen-2-yl)-oxazol-4-yl]-ethoxy}-benzyl)-[1,3]dioxane-2-carboxylic acid and its pharmaceutically acceptable salts;
2-Methyl-5-cis-{4-[2-(5-methyl-2-phenyl-oxazol-4-yl)-ethoxy]-benzyl}-[1,3]dioxane-2-carboxylic acid and its pharmaceutically acceptable salts;
2-Methyl-5-trans-{4-[2-(5-methyl-2-phenyl-oxazol-4-yl)-ethoxy]-benzyl}-[1,3]dioxane-2-carboxylic acid and its pharmaceutically acceptable salts;
2-Methyl-5-cis-{4-[3-(4-phenoxy-phenoxy)-propoxy]-benzyl}-[1,3]dioxane-2-carboxylic acid and its pharmaceutically acceptable salts;
5-cis-{4-[2-(4-Methanesulfonyloxy-phenyl)-ethoxy]-benzyl}-2-methyl-[1,3]dioxane-2-carboxylic acid and its pharmaceutically acceptable salts;
5-cis-[4-(2-tert-Butyl-5-methyl-oxazol-4-ylmethoxy)-benzyl]-2-methyl-[1,3]dioxane-2-carboxylic acid and its pharmaceutically acceptable salts;
2-Methyl-5-cis-{4-[4-methyl-2-(4-trifluoromethyl-phenyl)-thiazol-5-ylmethoxy]-benzyl}-[1,3]dioxane-2-carboxylic acid and its pharmaceutically acceptable salts;
2-Methyl-5-cis-{4-[2-(4-phenoxy-phenoxy)-ethoxy]-benzyl}-[1,3]dioxane-2-carboxylic acid and its pharmaceutically acceptable salts;
5-cis-[4-(2-Fluoro-benzyloxy)-benzyl]-2-methyl-[1,3]dioxane-2-carboxylic acid and its pharmaceutically acceptable salts;
2-Methyl-5-cis-(4-{2-[2-methyl-5-(4-methylsulfanyl-phenyl)-pyrrol-1-yl]-ethoxy}-benzyl)-[1,3]dioxane-2-carboxylic acid and its pharmaceutically acceptable salts;
5-cis-{4-[2-(2-tert-Butyl-5-methyl-oxazol-4-yd)-ethoxy]-benzyl}-2-methyl-[1,3]dioxane-2-carboxylic acid and its pharmaceutically acceptable salts;
5-cis-[6-(2-Fluoro-benzyloxy)-naphthalen-2-ylmethyl]-2-methyl-[1,3]dioxane-2-carboxylic acid and its pharmaceutically acceptable salts;
5-cis-[6-(Benzyloxy)-naphthalen-2-ylmethyl]-2-methyl-[1,3]dioxane-2-carboxylic acid and its pharmaceutically acceptable salts;
(Z)-2-Methyl-5-cis-{4-[2-(phenyl-pyridin-2-yl-methyleneaminooxy)-ethoxy]-benzyl}-[1,3]dioxane-2-carboxylic acid and its pharmaceutically acceptable salts;
(E)-2-Methyl-5-cis-{4-[2-(phenyl-pyridin-2-yl-methyleneaminooxy)-ethoxy]-benzyl}-[1,3]dioxane-2-carboxylic acid and its pharmaceutically acceptable salts;
5-cis-[4-(5-tert-Butyl-2-p-tolyl-2H-pyrazol-3-ylmethoxy)-benzyl]-2-methyl-[1,3]dioxane-2-carboxylic acid and its pharmaceutically acceptable salts;
2-Methyl-5-cis-{4-[2-(5-methyl-2-p-tolyl-oxazol-4-yl)-ethoxy]-benzyl}-[1,3]dioxane-2-carboxylic acid and its pharmaceutically acceptable salts;
2-Methyl-5-trans-[4-(5-methyl-2-phenyl-oxazol-4-ylmethoxy)-benzyl]-[1,3]dioxane-2-carboxylic acid and its pharmaceutically acceptable salts;
2-Methyl-5-trans-[4-(1-pyridin-2-yl-pyrrolidin-2-ylmethoxy)-benzyl]-[1,3]dioxane-2-carboxylic acid and its pharmaceutically acceptable salts;
2-Methyl-5-trans-{4-[2-(methyl-pyridin-2-yl-amino)-ethoxy]-benzyl}-[1,3]dioxane-2-carboxylic acid and its pharmaceutically acceptable salts;
2-Methyl-5-trans-[4-(5-methyl-2-p-tolyl-oxazol-4-ylmethoxy)-benzyl]-[1,3]dioxane-2-carboxylic acid and its pharmaceutically acceptable salts;
5-trans-[4-(2-Carbazol-9-yl-ethoxy)-benzyl]-2-methyl-[1,3]dioxane-2-carboxylic acid and its pharmaceutically acceptable salts;
5-trans-[4-(2-Indol-1-yl-ethoxy)-benzyl]-2-methyl-[1,3]dioxane-2-carboxylic acid and its pharmaceutically acceptable salts;
2-Methyl-5-trans-{4-[5-methyl-2-(5-methyl-thiophen-2-yl)-oxazol-4-ylmethoxy]-benzyl}-[1,3]dioxane-2-carboxylic acid and its pharmaceutically acceptable salts;
5-trans-{4-[2-(2,3-Dihydro-benzo[1,4]thiazin-4-yl)-ethoxy]-benzyl}-2-methyl-[1,3]dioxane-2-carboxylic acid and its pharmaceutically acceptable salts;
2-Methyl-5-trans-[4-(2-phenothiazin-10-yl-ethoxy)-benzyl]-[1,3]dioxane-2-carboxylic acid and its pharmaceutically acceptable salts;
5-trans-{4-[2-(4-Hexyl-3-oxo-3,4-dihydro-2H-benzo[1,4]oxazin-2-yl)-ethoxy]-benzyl}-2-methyl-[1,3]dioxane-2-carboxylic acid and its pharmaceutically acceptable salts;
2-Methyl-5-trans-(4-{2-[5-methyl-2-(5-methyl-thiophen-2-yl)-oxazol-4-yl]-ethoxy}-benzyl)-[1,3]dioxane-2-carboxylic acid and its pharmaceutically acceptable salts;
2-Methyl-5-trans-{4-[2-(5-methyl-2-phenyl-oxazol-4-yl)-ethoxy]-benzyl}-[1,3]dioxane-2-carboxylic acid and its pharmaceutically acceptable salts;
2-Methyl-5-trans-{4-[2-(5-methyl-2-phenyl-oxazol-4-yl)-ethoxy]-benzyl}-[1,3]dioxane-2-carboxylic acid and its pharmaceutically acceptable salts;
2-Methyl-5-trans-{4-[3-(4-phenoxy-phenoxy)-propoxy]-benzyl}-[1,3]dioxane-2-carboxylic acid and its pharmaceutically acceptable salts;
5-trans-{4-[2-(4-Methanesulfonyloxy-phenyl)-ethoxy]-benzyl}-2-methyl-[1,3]dioxane-2-carboxylic acid and its pharmaceutically acceptable salts;
5-trans-[4-(2-tert-Butyl-5-methyl-oxazol-4-ylmethoxy)-benzyl]-2-methyl-[1,3]dioxane-2-carboxylic acid and its pharmaceutically acceptable salts;
2-Methyl-5-trans-{4-[4-methyl-2-(4-trifluoromethyl-phenyl)-thiazol-5-ylmethoxy]-benzyl}-[1,3]dioxane-2-carboxylic acid and its pharmaceutically acceptable salts;
2-Methyl-5-trans-{4-[2-(4-phenoxy-phenoxy)-ethoxy]-benzyl}-[1,3]dioxane-2-carboxylic acid and its pharmaceutically acceptable salts;
5-trans-[4-(2-Fluoro-benzyloxy)-benzyl]-2-methyl-[1,3]dioxane-2-carboxylic acid and its pharmaceutically acceptable salts;
2-Methyl-5-trans-(4-{2-[2-methyl-5-(4-methylsulfanyl-phenyl)-pyrrol-1-yl]-ethoxy}-benzyl)-[1,3]dioxane-2-carboxylic acid and its pharmaceutically acceptable salts;
5-trans-{4-[2-(2-tert-Butyl-5-methyl-oxazol-4-yl)-ethoxy]-benzyl}-2-methyl-[1,3]dioxane-2-carboxylic acid and its pharmaceutically acceptable salts;
5-trans-[6-(2-Fluoro-benzyloxy)-naphthalen-2-ylmethyl]-2-methyl-[1,3]dioxane-2-carboxylic acid and its pharmaceutically acceptable salts;
5-trans-[6-(Benzyloxy)-naphthalen-2-ylmethyl]-2-methyl-[1,3]dioxane-2-carboxylic acid and its pharmaceutically acceptable salts;
(Z)-2-Methyl-5-trans-{4-[2-(phenyl-pyridin-2-yl-methyleneaminooxy)-ethoxy]-benzyl}-[1,3]dioxane-2-carboxylic acid and its pharmaceutically acceptable salts;
(E)-2-Methyl-5-trans-{4-[2-(phenyl-pyridin-2-yl-methyleneaminooxy)-ethoxy]-benzyl}-[1,3]dioxane-2-carboxylic acid and its pharmaceutically acceptable salts;
5-trans-[4-(5-tert-Butyl-2-p-tolyl-2H-pyrazol-3-ylmethoxy)-benzyl]-2-methyl-[1,3]dioxane-2-carboxylic acid and its pharmaceutically acceptable salts;
2-Methyl-5-trans-{4-[2-(5-methyl-2-p-tolyl-oxazol-4-yl)-ethoxy]-benzyl}-[1,3]dioxane-2-carboxylic acid and its pharmaceutically acceptable salts;
2-Methyl-5-cis-[4-(5-methyl-2-phenyl-oxazol-4-ylmethoxy)-phenyl]-[1,3]dioxane-2-carboxylic acid and its pharmaceutically acceptable salts;
2-Methyl-5-cis-{4-[2-(5-methyl-2-phenyl-oxazol-4-yl)-ethoxy]-phenyl}-[1,3]dioxane-2-carboxylic acid and its pharmaceutically acceptable salts;
2-Methyl-5-cis-[4-(2-phenoxazin-10-yl-ethoxy)-phenyl]-[1,3]dioxane-2-carboxylic acid and its pharmaceutically acceptable salts;
2-Methyl-5-cis-[4-(3-methyl-4-oxo-3,4-dihydro-quinazolin-2-ylmethoxy)-phenyl]-[1,3]dioxane-2-carboxylic acid and its pharmaceutically acceptable salts;
5-cis-[4-(2-Indol-1-yl-ethoxy)-phenyl]-2-methyl-[1,3]dioxane-2-carboxylic acid and its pharmaceutically acceptable salts;
5-cis-{4-[2-(5-Ethyl-pyridin-2-yl)-ethoxy]-phenyl}-2-methyl-[1,3]dioxane-2-carboxylic acid and its pharmaceutically acceptable salts;
2-Methyl-5-cis-[4-(5-methyl-2-p-tolyl-oxazol-4-ylmethoxy)-phenyl]-[1,3]dioxane-2-carboxylic acid and its pharmaceutically acceptable salts;
2-Methyl-5-cis-{4-[2-(5-methyl-2-p-tolyl-oxazol-4-yl)-ethoxy]-phenyl}-[1,3]dioxane-2-carboxylic acid and its pharmaceutically acceptable salts;
5-cis-{4-[2-(4-Methanesulfonyloxy-phenyl)-ethoxy]-phenyl}-2-methyl-[1,3]dioxane-2-carboxylic acid and its pharmaceutically acceptable salts;
2-Methyl-5-trans-[4-(5-methyl-2-phenyl-oxazol-4-ylmethoxy)-phenyl]-[1,3]dioxane-2-carboxylic acid and its pharmaceutically acceptable salts;
2-Methyl-5-trans-[4-(5-methyl-2-p-tolyl-oxazol-4-ylmethoxy)-phenyl]-[1,3]dioxane-2-carboxylic acid and its pharmaceutically acceptable salts;
2-Methyl-5-trans-{4-[2-(5-methyl-2-phenyl-oxazol-4-yl)-ethoxy]-phenyl}-[1,3]dioxane-2-carboxylic acid and its pharmaceutically acceptable salts;
2-Methyl-5-trans-{4-[2-(5-methyl-2-p-tolyl-oxazol-4-yl)-ethoxy]-phenyl}-[1,3]dioxane-2-carboxylic acid and its pharmaceutically acceptable salts;
2-Methyl-5-trans-{4-[4-methyl-2-(4-trifluoromethyl-phenyl)-thiazol-5-ylmethoxy]-phenyl}-[1,3]dioxane-2-carboxylic acid and its pharmaceutically acceptable salts;
2-Methyl-5-cis-{4-[4-methyl-2-(4-trifluoromethyl-phenyl)-thiazol-5-ylmethoxy]-phenyl}-[1,3]dioxane-2-carboxylic acid and its pharmaceutically acceptable salts;
2-Methyl-5-cis-(4-{2-[2-methyl-5-(4-methylsulfanyl-phenyl)-pyrrol-1-yl]-ethoxy}-phenyl)-[1,3]dioxane-2-carboxylic acid and its pharmaceutically acceptable salts;
5-cis-[4-(2-tert-Butyl-5-methyl-oxazol-4-ylmethoxy)-phenyl]-2-methyl-[1,3]dioxane-2-carboxylic acid and its pharmaceutically acceptable salts;
5-trans-[4-(2-tert-Butyl-5-methyl-oxazol-4-ylmethoxy)-phenyl]-2-methyl-[1,3]dioxane-2-carboxylic acid and its pharmaceutically acceptable salts;
2-Methyl-5-cis-{4-[2-(4-phenoxy-phenoxy)-ethoxy]-phenyl}-[1,3]dioxane-2-carboxylic acid and its pharmaceutically acceptable salts;
2-Methyl-5-trans-{4-[2-(4-phenoxy-phenoxy)-ethoxy]-phenyl}-[1,3]dioxane-2-carboxylic acid and its pharmaceutically acceptable salts;
5-cis-[4-(2-Fluoro-benzyloxy)-phenyl]-2-methyl-[1,3]dioxane-2-carboxylic acid and its pharmaceutically acceptable salts;
5-trans-[4-(2-Fluoro-benzyloxy)-phenyl]-2-methyl-[1,3]dioxane-2-carboxylic acid and its pharmaceutically acceptable salts;
5-cis-[4-(5-tert-Butyl-2-p-tolyl-2H-pyrazol-3-ylmethoxy)-phenyl]-2-methyl-[1,3]dioxane-2-carboxylic acid and its pharmaceutically acceptable salts;
5-trans-[4-(5-tert-Butyl-2-p-tolyl-2H-pyrazol-3-ylmethoxy)-phenyl]-2-methyl-[1,3]dioxane-2-carboxylic acid and its pharmaceutically acceptable salts;
2-Methyl-5-trans-[4-(2-oxo-3-phenyl-oxazolidin-5-ylmethoxy)-phenyl]-[1,3]dioxane-2-carboxylic acid and its pharmaceutically acceptable salts;
2-Methyl-5-cis-[4-(2-oxo-3-phenyl-oxazolidin-5-ylmethoxy)-phenyl]-[1,3]dioxane-2-carboxylic acid and its pharmaceutically acceptable salts;
5-trans-{4-[2-(4-Methanesulfonyloxy-phenyl)-ethoxy]-phenyl}-2-methyl-[1,3]dioxane-2-carboxylic acid and its pharmaceutically acceptable salts;
2-Methyl-5-trans-[4-(3-methyl-4-oxo-3,4-dihydro-quinazolin-2-ylmethoxy)-phenyl]-[1,3]dioxane-2-carboxylic acid and its pharmaceutically acceptable salts.

13. A pharmaceutical composition which comprises compounds of formula (I), as claimed in claim 1 and a pharmaceutically acceptable carrier, diluent or excipients.

14. A method of preventing or treating diseases caused by hyperlipidaemia, hypercholesteremia, hyperglycemia, obesity, impaired glucose tolerance, leptin resistance, insulin resistance, diabetic complications, comprising administering an effective, non-toxic amount of compound of formula (I) or suitable pharmaceutical composition containing said compound of formula (I) as claimed in claim 1, to a patient in need thereof.

15. The method according to claim 1, wherein the disease is type 2 diabetes, impaired glucose tolerance, dyslipidaemia, hypertension, obesity, atherosclerosis, hyperlipidaemia, coronary artery disease, cardiovascular disorders and other diseases wherein insulin resistance is the underlying pathophysiological mechanism.

16. A medicine for treating/reducing any of the disease conditions described in any preceding claims which comprises administering a compound of formula (I), as defined in claim 1 and a pharmaceutically acceptable carrier, diluent or excipients to a patient in need thereof.

17. Use of compounds of formula (I), their pharmaceutical compositions and medicines containing them as defined in claim 1 as a medicament suitable for the treatment of diseases mentioned in claim 1.

18. A process for preparing compound of formula (I) as claimed in claim 1 comprising the steps of
i) reduction of compound of formula (II) wherein all the symbols are as defined earlier to compound of formula (III) wherein all the symbols are as defined earlier; 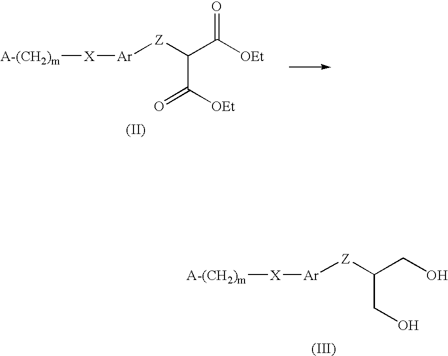
ii) reacting compound of formula (III) wherein all the symbols are as defined earlier with suitable ketoester of the formula RC(O)Y wherein R is as defined earlier and Y is COOR¹ where R¹ is alkyl or aryl to yield compound of formula (Ia) wherein Y represents COOR¹ where R¹ is alkyl or aryl and all other symbols are as defined earlier; 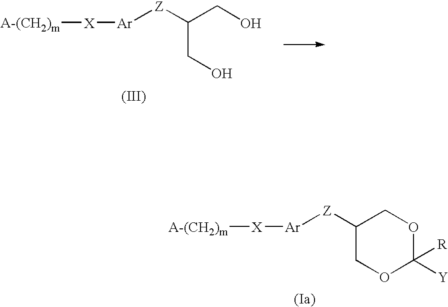
iii) hydrolysis of compound of general formula (Ia) wherein Y is COOR¹ where R¹ is alkyl or aryl and all other symbols are as defined earlier to yield compound of general formula (I) wherein Y is COOH and all other symbols are as defined earlier.

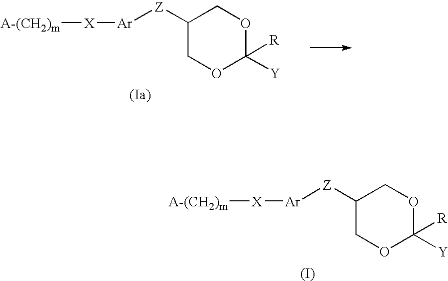

19. A process for preparing compounds of formula (I) as claimed in claim 1 comprising
i) reacting compounds of general formula (IV) where all symbols are as defined earlier and ‘L’ represents a suitable leaving group preferably selected from halogen, mesylate, tosylate or triflate with compounds of general formula (V), where all symbols are as defined earlier and Y represent COOR¹ where R¹ represents optionally substituted alkyl or aryl groups to yield compound of general formula (Ia) where all symbols are as defined earlier; 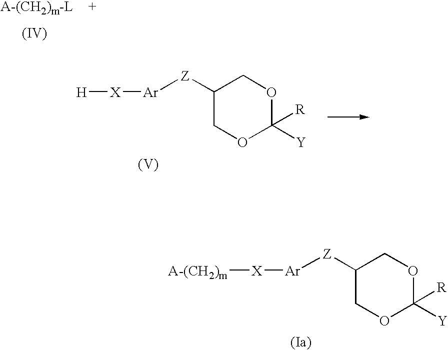
ii) hydrolysis of compound of general formula (Ia) wherein ‘Y’ is COOR¹ where R¹ represents optionally substituted alkyl or aryl groups and all other symbols are as defined earlier to yield compound of general formula (I) where in ‘Y’ is COOH and all other symbols are as defined earlier. [chemical structure]
